(19) United States
(12) Patent Application Publication
(10) Pub. No.: US 2014/0018538 A1
(43) Pub. Date: Jan. 16, 2014

(54) HIGH-VALENT PALLADIUM FLUORIDE COMPLEXES AND USES THEREOF

(71) Applicants: Eunsung Lee, Pohang-si (KR); Adam Seth Kamlet, Mystic, CT (US); David C. Powers, Belmont, MA (US); Takeru Furuya, Koganei-shi (JP); Tobias Ritter, Cambridge, MA (US)

(72) Inventors: Eunsung Lee, Pohang-si (KR); Adam Seth Kamlet, Mystic, CT (US); David C. Powers, Belmont, MA (US); Takeru Furuya, Koganei-shi (JP); Tobias Ritter, Cambridge, MA (US)

(73) Assignee: PRESIDENT AND FELLOWS OF HARVARD COLLEGE, Cambridge, MA (US)

(21) Appl. No.: 13/817,874

(22) PCT Filed: Aug. 19, 2011
(86) PCT No.: PCT/US11/48451

§ 371 Date: Oct. 3, 2013

Related U.S. Application Data
(60) Provisional application No. 61/375,652, filed on Aug. 20, 2010.
(60) Provisional application No. 61/508,586, filed on Jul. 15, 2011.

(51) Int. Cl. C07F 15/00; C07J 1/00; C07C 41/22

(52) U.S. Cl. CPC C07F 15/006; C07C 41/22; C07J 1/00
USPC 546/4; 568/647; 552/651

(57) ABSTRACT

The present invention provides novel high-valent palladium complexes. The complexes typically include multi-dentate ligands that stabilize the octahedral coordination sphere of the palladium(IV) atom. These complexes are useful in fluorinating organic compounds and preparing high-valent palladium fluoride complexes. The invention is particularly useful for fluorinating compounds with ¹⁹F for PET imaging.

### RELATED APPLICATIONS

The present application claims priority under 35 U.S.C. §119(e) to U.S. provisional applications: U.S. Ser. No. 61/508,586, filed Jul. 15, 2011; and U.S. Ser. No. 61/375,652, filed Aug. 20, 20, 2010; each of which is incorporated herein by reference.

### BACKGROUND OF THE INVENTION

The regioselective fluorination of organic compounds is an important challenge in the synthesis of pharmaceuticals and agrochemicals (see, for example, Muller et al., Science 2007, 317, 1881-1886; Park et al., Annual Review of Pharmacology and Toxicology 2001, 41, 443-470; Bohm et al., ChemBioChem 2004, 5, 637-643; and Jeschke, ChemBioChem. 2004, 5, 570-589).

Syntheses of simple fluoroarenes currently rely on the pyrolysis of diazonium tetrafluoroborates (Balz, G.; Schiemann, G. Ber. Deut. Chem. Ges. 1927, 60, 1186-1190), direct fluorination using highly reactive, elemental fluorine (Sandford, J. Fluorine Chem. 2007, 128, 90-104), or nucleophilic aromatic substitution reactions of electron-poor aromatic systems by displacement of other halogens or nitro groups (Sun et al., Angew. Chem., Int. Ed. 2006, 45, 2720-2725; Adams et al., Chem. Soc. Rev. 1999, 28, 225-231). The reductive elimination of arylfluorides from palladium(II) fluoride complexes is an attractive potential alternative that has been investigated by Grushin (Grushin, Chem.—Eur. J. 2002, 8, 1006-1014) over the past decade and more recently by Yandulov. A single substrate—p-fluoronitrobenzene—has been prepared successfully in 10% yield in the Yandulov study from a stoichiometric palladium fluoride complex (Yandulov et al., J. Am. Chem. Soc. 2007, 129, 1342-1358) (See also Watson et al., Science, 2009, Vol. 325. No. 5948, pp. 1661-1664). Directed electrophilic fluorination of phenylpyridine derivatives and related structures using catalytic palladium(II) acetate and N-fluoropyridinium salts has been reported by Sanford in 2006 (Hull et al., J. Am. Chem. Soc. 2006, 128, 7134-7135). Taking advantage of the directing effect of a pyridine substituent, proximal carbon-hydrogen bonds can be fluorinated using microwave irradiation at high temperatures (100-150° C., 1-4 h, 33-75% yield). However, the fact that there is an absence in the literature of any general, functional-group-tolerant fluorination reaction methodology reflects the difficulty of forming carbon-fluorine bonds.

The use of ¹⁸F-labelled organic compounds for positron-emission tomography (PET) requires the controlled, efficient introduction of fluorine into functionalized molecules (see, for example, Couturier et al., Eur. J. Nucl. Med. Mol. Imaging. 2004, 31, 1182-1206; Lasne et al., “Chemistry of beta(+)-emitting compounds based on fluorine-18” In Contrast Agents II, 2002; Vol. 222, pp 201-258; and Phelps, Proc. Natl. Acad. Sci. U.S.A. 2000, 97,9226-9233). PET has been used to measure presynaptic accumulation of ¹⁸F-fluorodopa tracer in the dopaminergic regions of the brain (see, for example, Ernst et al., “Presynaptic Dopaminergic Deficits in Lesch-Nyhan Disease” New England Journal of Medicine (1996) 334:1568-1572), but fluorination of other organic compounds has been difficult due to lack of an appropriate fluorination method.

Despite the utility of fluorinated organic compounds in multiple pharmaceutical, diagnostic, and agrochemical applications, C—F bond formation remains a challenging organic transformation with no broadly applicable solutions.

### SUMMARY OF THE INVENTION

The present invention provides novel high-valent palladium complexes and methods of using these complexes in the fluorination of organic compounds. The inventive system is also useful in preparing high-valent palladium fluoride complexes which may then be employed in the fluorination of a variety of organic compounds. The inventive system is also particularly useful in preparing ¹⁸F-labeled compounds for PET imaging. The complexes are typically palladium(IV) fluoride complexes as described herein. The complexes include two fixed ligands that stabilize the high-valent palladium complex. In certain embodiments, one of the ligands is a bidentate ligand, and the other ligand is a tridentate ligand. The inventive system relies on the transfer of electrophilic fluorine, which is analogous to the commercially available fluorinating reagent, Selectfluor® (N-chloromethyl-N′-fluorotriethylenediammonium bis(tetrafluoroborate)).

In one aspect, the present invention is directed to a palladium complex of formula (VII):

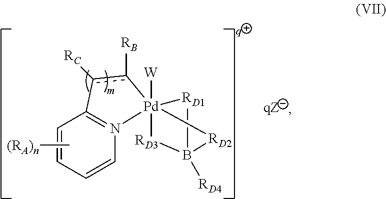

wherein:

the dashed line represents the presence or absence of a bond;

Pd is in the oxidation state +IV;

W is Br, hydroxyl, alkoxy, aryloxy, —NO₃, nitro, —N₃, ClO₄, PO₄, SO₄, —OSO₂-aryl, heteroaryl or heterocyclyl, each of which is substituted with p occurrences of R_{F};

n is 0, 1, 2, 3 or 4;

m is 0, 1, 2 or 3;

p is 0, 1, 2 or 3;

q is 1 or 2;

each occurrence of R_{A} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; wherein two R_{A} may be taken together with the atoms to which they are attached to form a substituted or unsubstituted carbocyclic, heterocyclic, aryl or heteroaryl ring;

each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R_{C} is independently hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; wherein R_{C} and R_{B} may be taken together with the atoms to which they are attached to form a substituted or unsubstituted heterocyclic or heteroaryl ring; and wherein R_{C} and R_{A} may be taken together with the atoms to which they are attached to form a substituted or unsubstituted carbocyclic, heterocyclic, aryl or heteroaryl ring;

R_{D1}, R_{D2}, R_{D3}, and R_{D4} are each independently cyclic or acyclic, branched or unbranched aliphatic; cyclic or acyclic, branched or unbranched heteroaliphatic; branched or unbranched aryl; branched or unbranched heteroaryl, each of which is substituted with 0-3 occurences of R_{H};

each occurrence of R_{H} is independently hydrogen, halogen, alkyl, alkoxy, aryl or heteroaryl;

each occurrence of R_{F} is independently halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an aryl moiety; a heteroaryl moiety; alkoxy; aryloxy; alkylthio; arylthio; amino, alkylamino, dialkylamino, heteroaryloxy; or heteroarylthio moiety; and

Z⁻ is an anion.

In some embodiments, Z is a halide, acetate, tosylate, azide, tetrafluoroborate, tetraphenylborate, tetrakis(pentafluorophenyl)borate, [B[3,5-(CF₃)₂C₆H₃]₄], hexafluorophosphate, phosphate, sulfate, perchlorate, trifluoromethanesulfonate or hexafluoroantimonate. In some embodiments, Z is trifluoromethanesulfonate. In some embodiments, Z is hexafluoroarsenate.

In some embodiments, R_{C} is hydrogen.

In some embodiments, n is 0. In some embodiments, n is 1. In some embodiments, n is 2. In some embodiments, m is 1. In some embodiments, p is 0. In some embodiments, q is 1. In some embodiments, q is 2.

In some embodiments, R_{A} and R_{C} taken together with the atoms to which they are attached form an aryl ring. In some embodiments, R_{A} and R_{C} taken together with the atoms to which they are attached form a phenyl ring. In some embodiments, R_{B} and R_{C} taken together with the atoms to which they are attached form an aryl ring. In some embodiments, R_{B} and R_{C} taken together to form a phenyl ring.

In some embodiments, the dashed line represents the absence of a bond. In some embodiments, the dashed line represents the presence of a bond.

In some embodiments, R_{D1}, R_{D2}, R_{D3} and R_{D4} are each a 5-membered heteroaryl ring. In some embodiments, R_{D1}, R_{D2}, R_{D3} and R_{D4} are each a pyrazolyl ring substituted with 0-3 occurrences of R^{H}. In some embodiments, R_{D1}, R_{D2}, R_{D3} and R_{D4} are each an unsubstituted pyrazolyl ring. In some embodiments, R_{D1}, R_{D2}, R_{D3} and R_{D4} are each a pyrazolyl ring substituted with 1 occurrence of R^{H}. In some embodiments, each R^{H} is chloro.

In some embodiments, W is Br, hydroxyl, alkoxy, aryloxy, —NO₃, nitro, —N₃, ClO₄, PO₄, SO₄, —OSO₂-aryl, an N-containing heteroaryl or an N-containing heterocyclyl. In some embodiments, W is Br. In some embodiments, W is hydroxyl. In some embodiments, W is —NO₃. In some embodiments, W is —N₃. In some embodiments, W is PO₄. In some embodiments, W is SO₄. In some embodiments, W is ClO₄. In some embodiments, W is —OSO₂-aryl (e.g., —OSO₂-phenyl or —OSO₂-tolyl). In some embodiments, W is aryloxy (e.g., phenoxy or 2,4,6-trimethylphenyoxy). In some embodiments, W is heterocyclyl (e.g., an optionally substituted N-containing heterocyclyl). In some embodiments, W is heteroaryl (e.g., an optionally substituted N-containing heteroaryl). In some embodiments, the palladium complex is selected from a complex of formula (IX):

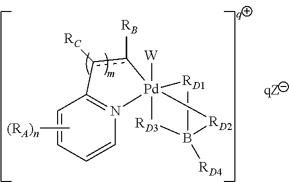

wherein:

the dashed line represents the presence or absence of a bond;

Pd is in the oxidation state +IV;

T is Br, hydroxyl, aryloxy, —NO₃, nitro, —N₃, ClO₄, PO₄, SO₄, or —O—SO₂-aryl; and

n, m, q, R_{A}, R_{B}, R_{C}, R_{D1}, R_{D2}, R_{D3}, R_{D4}, R_{H}, R″, R_{F}, and Z are as defined in formula (VII).

In some embodiments, R_{C} is hydrogen.

In some embodiments, n is 0. In some embodiments, n is 1. In some embodiments, m is 1. In some embodiments, q is 1.

In some embodiments, R_{A} and R_{C} taken together with the atoms to which they are attached form an aryl ring. In some embodiments, R_{A} and R_{C} taken together with the atoms to which they are attached form a phenyl ring. In some embodiments, R_{B} and R_{C} taken together with the atoms to which they are attached form an aryl ring. In some embodiments, R_{B} and R_{C} taken together to form a phenyl ring.

In some embodiments, the dashed line represents the absence of a bond. In some embodiments, the dashed line represents the presence of a bond.

In some embodiments, R_{D1}, R_{D2}, R_{D3} and R_{D4} are each a 5-membered heteroaryl ring. In some embodiments, R_{D1}, R_{D2}, R_{D3} and R_{D4} are each a pyrazolyl ring 0-3 occurrences of R^{H}. In some embodiments, R_{D1}, R_{D2}, R_{D3} and R_{D4} are each an unsubstituted pyrazolyl ring. In some embodiments, R_{D1}, R_{D2}, R_{D3} and R_{D4} are each a pyrazolyl ring substituted with 1 occurrence of R^{H}. In some embodiments, each R^{H} is halogen (e.g., 4-chloro).

In some embodiments, Z is trifluoromethanesulfonate.

In some embodiments, the compound of formula (IX) is selected from the following:

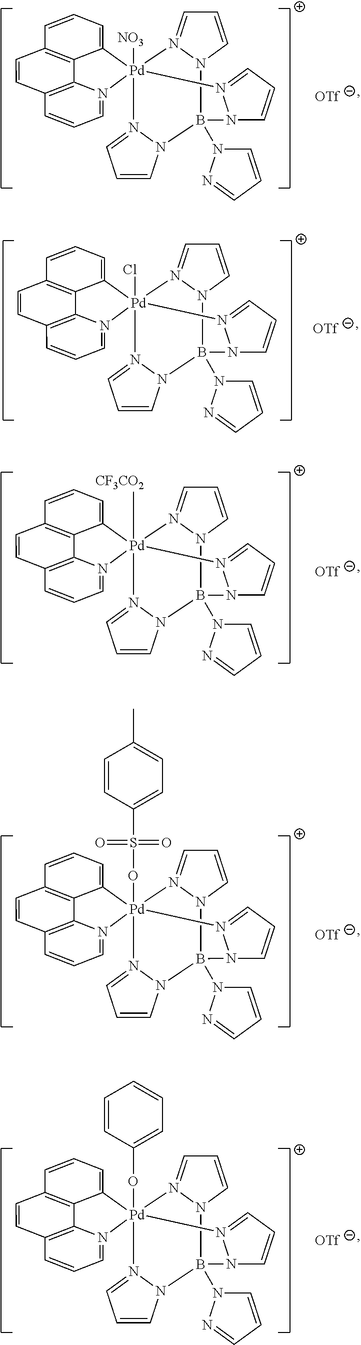

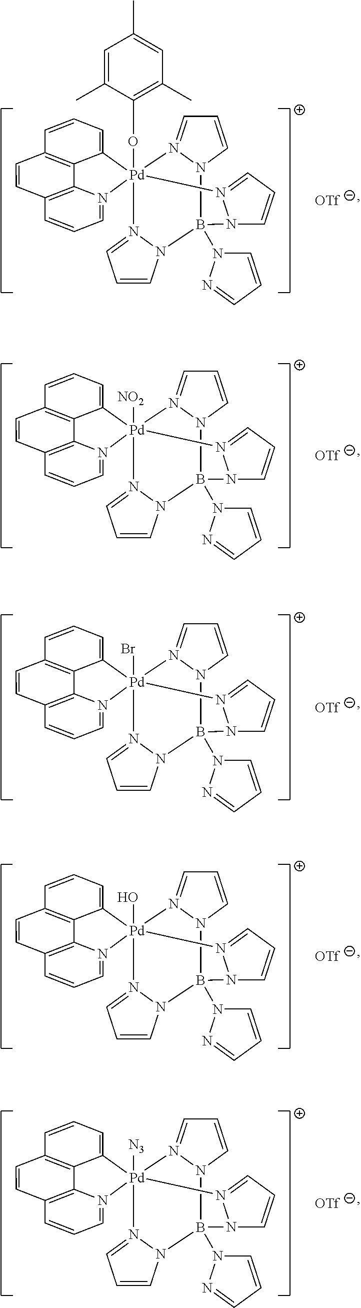

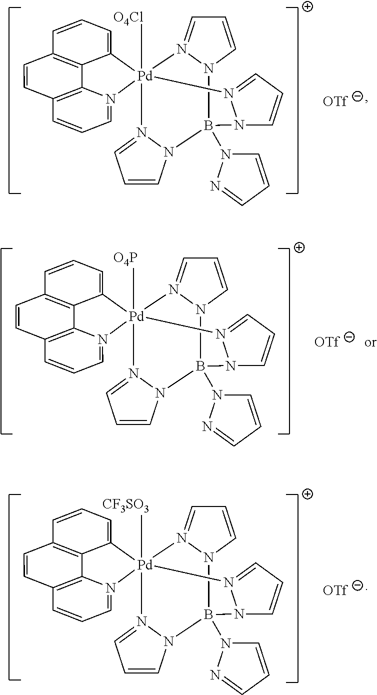

In some embodiments, the palladium complex is selected from a complex of formula (I):

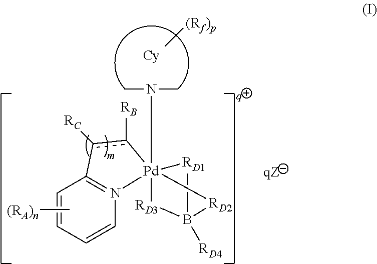

wherein:

the dashed line represents the presence or absence of a bond;

Pd is in the oxidation state +IV;

Cy taken together with the nitrogen atom to which it is attached forms a heterocyclyl or heteroaryl ring;

n, m, p, q, R_{A}, R_{B}, R_{C}, R_{D1}, R_{D2}, R_{D3}, R_{D4}, R_{F}, R_{H}, R″ and Z are as defined in formula (VII).

In some embodiments, Z is a halide, acetate, tosylate, azide, tetrafluoroborate, tetraphenylborate, tetrakis(pentafluorophenyl)borate, [B[3,5-(CF₃)₂C₆H₃]₄]⁻, hexafluorophosphate, phosphate, sulfate, perchlorate, trifluoromethanesulfonate or hexafluoroantimonate.

In some embodiments, Cy taken together with the nitrogen to which it is attached forms a heteroaryl ring. In some embodiments, Cy taken together with the nitrogen to which it is attached forms a pyridyl ring.

In some embodiments, R_{C} is hydrogen.

In some embodiments, n is 0. In some embodiments, n is 1. In some embodiments, m is 1. In some embodiments, p is 0. In some embodiments, q is 2.

In some embodiments, R_{A} and R_{C} taken together with the atoms to which they are attached form an aryl ring. In some embodiments, R_{A} and R_{C} taken together with the atoms to which they are attached form a phenyl ring. In some embodiments, R_{B} and R_{C} taken together with the atoms to which they are attached form an aryl ring. In some embodiments,

R_{B} and R_{C} taken together to form a phenyl ring.

In some embodiments, the dashed line represents the absence of a bond. In some embodiments, the dashed line represents the presence of a bond.

In some embodiments, R_{D1}, R_{D2}, R_{D3} and R_{D4} are each a 5-membered heteroaryl ring. In some embodiments, R_{D1}, R_{D2}, R_{D3} and R_{D4} are each a pyrazolyl ring 0-3 occurrences of R^{H}. In some embodiments, R_{D1}, R_{D2}, R_{D3} and R_{D4} are each an unsubstituted pyrazolyl ring. In some embodiments, R_{D1}, R_{D2}, R_{D3} and R_{D4} are each a pyrazolyl ring substituted with 1 occurrence of R^{H}. In some embodiments, each R^{H} is halogen (e.g., 4-chloro).

In some embodiments, Z is trifluoromethanesulfonate.

In some embodiments, the palladium complex of formula (I) is selected from a complex of formula (Ia):

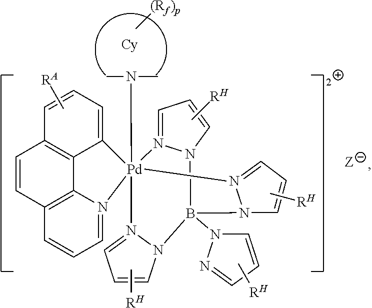

wherein

p, R^{A}, R^{F}, R^{H} and Z are as defined in formula (I). In some embodiments, the palladium complex is selected from one of the following:

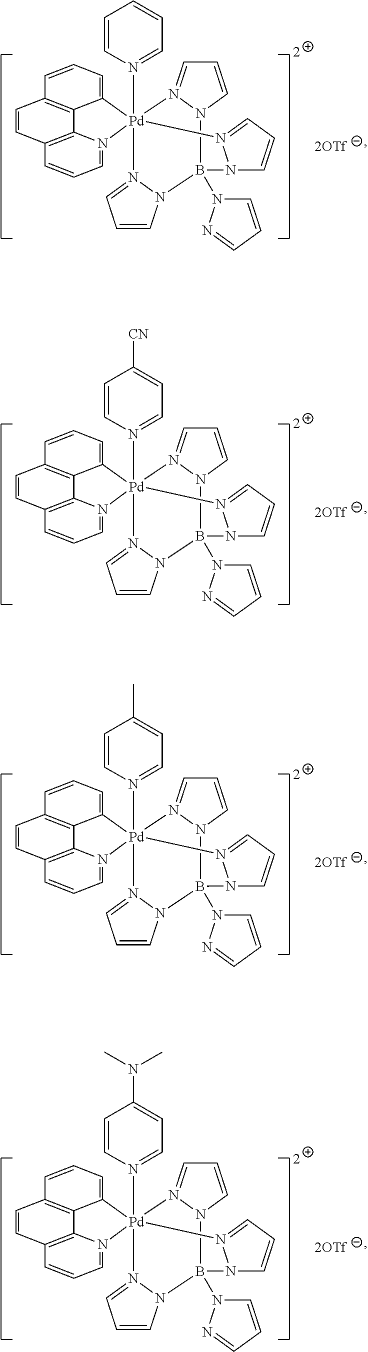

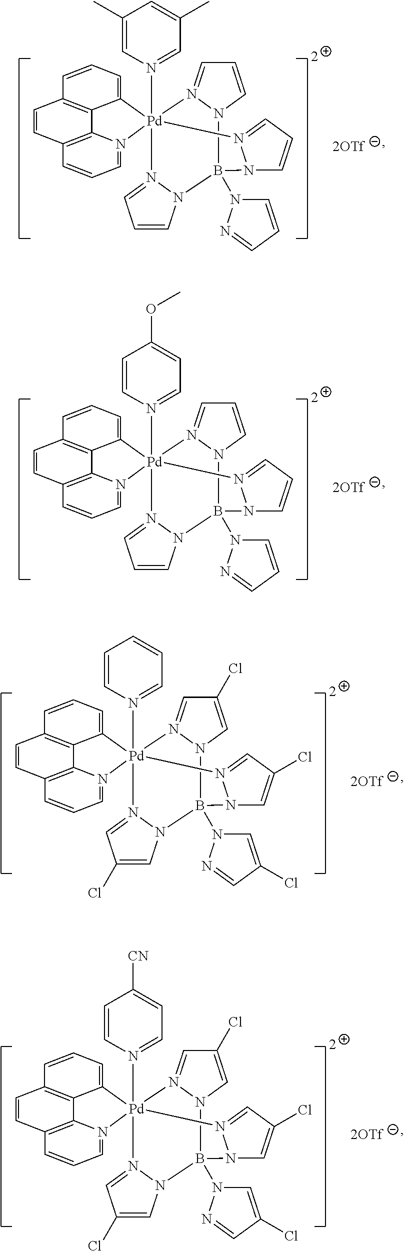

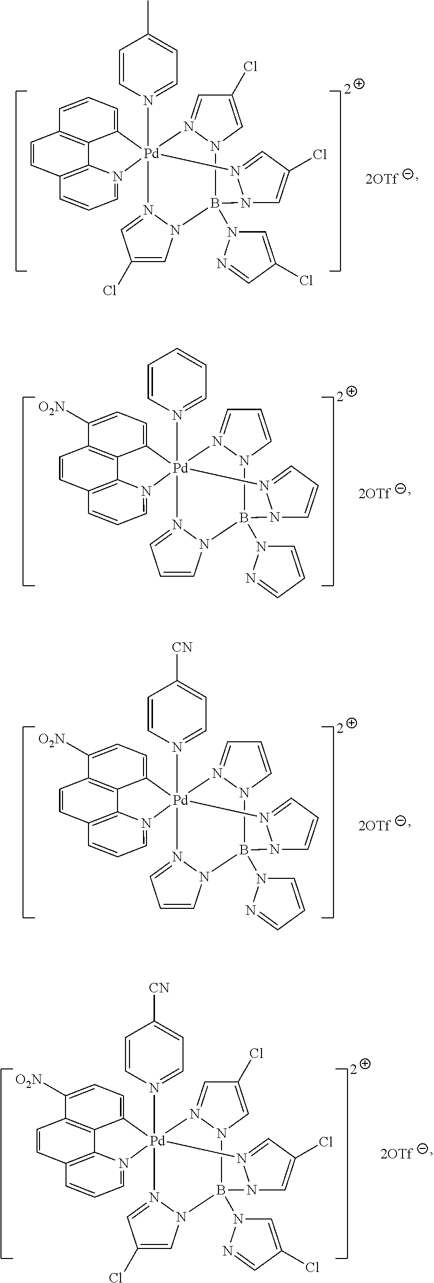

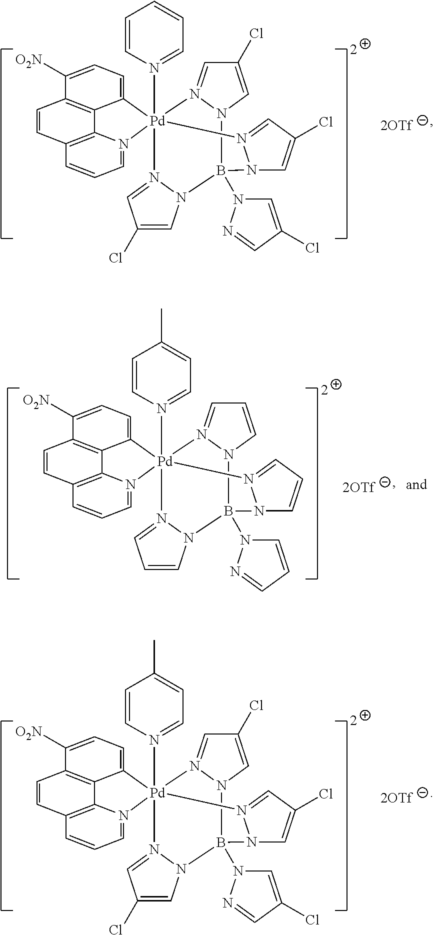

In some embodiments, the palladium complex has the following formula:

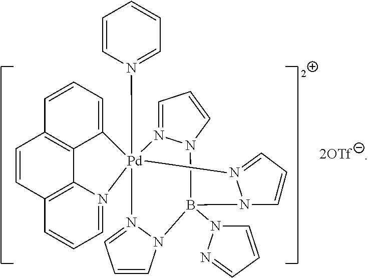

In some embodiments, the palladium complex has the following formula:

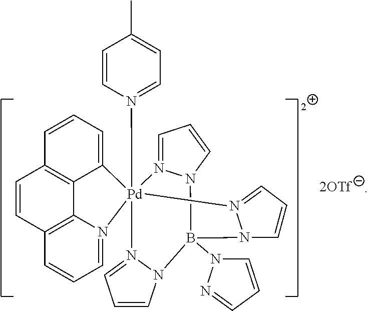

In another aspect, the present invention is directed to a palladium complex of formula (II):

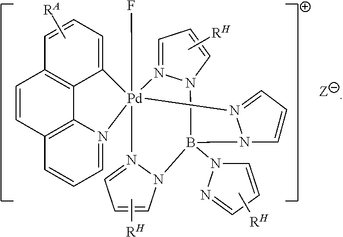

wherein:

R^{A} is as defined for formula (VII);

each R^{H} is independently selected from hydrogen, halogen, alkyl, alkoxy, aryl or heteoraryl;

F is comprises ¹⁸F or ¹⁹F; and

Z is an anion.

In some embodiments, R^{A} is nitro.

In some embodiments, each R^{H} is hydrogen.

In some embodiments, each R^{H} is halogen (e.g., chloro).

In some embodiments, Z is a halide, acetate, tosylate, azide, tetrafluoroborate, tetraphenylborate, tetrakis(pentafluorophenyl)borate, [B[3,5-(CF₃)₂C₆H₃]₄]⁻, hexafluorophosphate, phosphate, sulfate, perchlorate, trifluoromethanesulfonate or hexafluoroantimonate. In some embodiments, Z is trifluoromethanesulfonate.

In some embodiments, F comprises ¹⁸F. In some embodiments, F comprises ¹⁹F.

In some embodiments, the palladium complex of formula (II) is selected from the following:

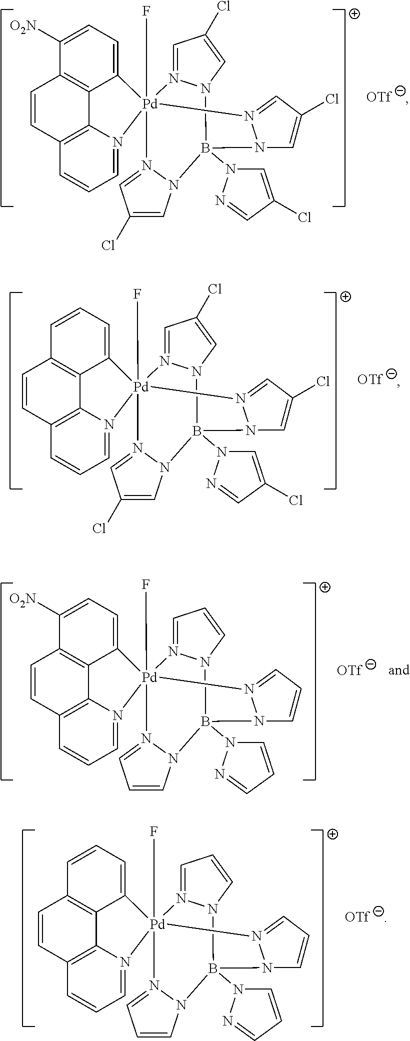

In some embodiments, the palladium complex of formula (II) is

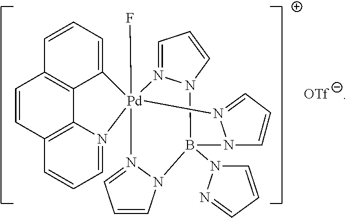

In some embodiments, the palladium complex of formula (II) is

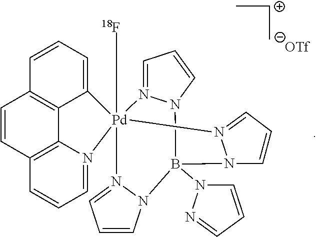

In another aspect, the present invention is directed to a palladium complex of formula (VIII):

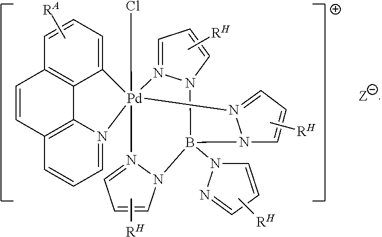

wherein:

R^{A}, R^{H} and Z are as defined in formula (II).

In some embodiments, R^{A} is nitro.

In some embodiments, each R^{H} is independently hydrogen. In some embodiments, each R^{H} is independently halogen (e.g., chloro).

In some embodiments, the palladium complex of formula (VIII) is

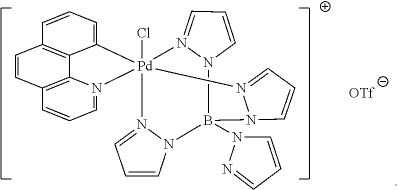

In another aspect, the present invention is directed to a method of generating an electrophilic fluorinating reagent. The method comprises treating a composition of F— with a palladium complex of formula (VII), thereby generating the electrophilic fluorinating reagent.

In some embodiments, the palladium complex of formula (VII) is selected from a complex of formula (I). In some embodiments, the palladium complex of formula (VII) is selected from a complex of formula (IX).

In some embodiments, the composition further comprises a solvent. In some embodiments, the solvent is a polar aprotic solvent. In some embodiments, the solvent is a nonpolar solvent. In some embodiments, the solvent is acetone. In some embodiments, the solvent is methylene chloride. In some embodiments, the solvent is tetrahydrofuran. In some embodiments, the solvent is benzene. In some embodiments, the solvent is acetonitrile. In some embodiments, the solvent is 1,2-dichloroethane.

In some embodiments, the composition further comprises a base. In some embodiments, the composition further comprises potassium bicarbonate.

In some embodiments, F comprises ¹⁸F. In some embodiments, F comprises ¹⁹F. In some embodiments, the composition further comprises a phase transfer catalyst. In some embodiments, the phase transfer catalyst is a crown ether. In some embodiments, the crown ether is 18-crown-6.

In some embodiments, the method is carried out under an inert atmosphere.

In some embodiments, the method is performed under anhydrous conditions.

In some embodiments, the method is carried out in the presence of a source of energy. In some embodiments, the source of energy is heat.

In another aspect, the present invention is directed to a method of converting F— to an electrophilic fluorinating reagent, the method comprising treating a composition of F— with a palladium complex of formula (VIII), thereby converting the F to an electrophilic fluorinating reagent.

In some embodiments, the composition further comprises a solvent. In some embodiments, the solvent is a polar aprotic solvent. In some embodiments, the solvent is a nonpolar solvent. In some embodiments, the solvent is acetone. In some embodiments, the solvent is methylene chloride. In some embodiments, the solvent is tetrahydrofuran. In some embodiments, the solvent is benzene. In some embodiments, the solvent is acetonitrile. In some embodiments, the solvent is 1,2-dichloroethane.

In some embodiments, the composition further comprises a base. In some embodiments, the composition further comprises potassium bicarbonate. In some embodiments, F comprises ¹⁸F. In some embodiments, F comprises ¹⁹F.

In some embodiments, the composition further comprises a phase transfer catalyst. In some embodiments, the phase transfer catalyst is a crown ether. In some embodiments, the crown ether is 18-crown-6.

In some embodiments, the method is carried out under an inert atmosphere.

In some embodiments, the method is performed under anhydrous conditions.

In some embodiments, the method is carried out in the presence of a source of energy. In some embodiments, the source of energy is heat.

In another aspect, the present invention is directed to a method of making a palladium complex of formula (I), the method comprising treating a palladium complex of formula (III):

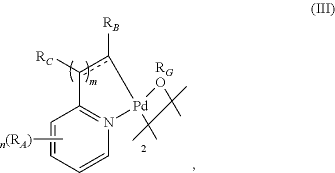

with a borate complex of formula (IV):

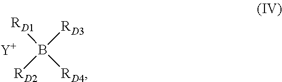

to provide a compound of formula (V):

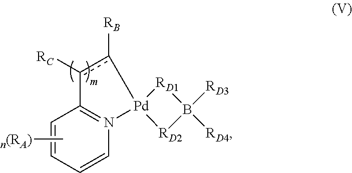

the method further comprising, treating a compound of formula (V) with a compound of formula (VI):

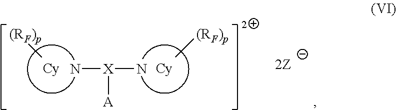

to provide a compound of formula (I), wherein
A is an aryl or heteroaryl group;
R_{G} is acyl;
Y⁺ is a cation;
X is a halogen; and
R_{A}, R_{B}, R_{C}, R_{D1}, R_{D2}, R_{D3}, R_{D4}, R_{F}, Z, Cy, n, m and p are as defined for formula (I).

In some embodiments, X is iodine.

In some embodiments, Y is potassium.

In some embodiments, Cy is pyridinyl.

In some embodiments, R_{C} is hydrogen.

In some embodiments, n is 0. In some embodiments, n is 1. In some embodiments, m is 1. In some embodiments, p is 0. In some embodiments, p is 1. In some embodiments, p is 2.

In some embodiments, R_{A} and R_{C} taken together with the atoms to which they are attached form an aryl ring. In some embodiments, R_{A} and R_{C} taken together with the atoms to which they are attached form a phenyl ring. In some embodiments, R_{B} and R_{C} taken together with the atoms to which they are attached form an aryl ring. In some embodiments, R_{B} and R_{C} taken together with the atoms to which they are attached form a phenyl ring.

In some embodiments, each R_{F} is independently unsubstituted alkyl (e.g., methyl). In some embodiments, each R_{F} is independently —CN.

In some embodiments, the dashed line represents the absence of a bond. In some embodiments, the dashed line represents the presence of a bond.

In some embodiments, R_{D1}, R_{D2}, R_{D3} and R_{D4} are each a 5-membered heteroaryl ring. In some embodiments, R_{D1}, R_{D2}, R_{D3} and R_{D4} are each a pyrazolyl ring. In some embodiments, R_{D1}, R_{D2}, R_{D3} and R_{D4} are each an unsubstituted pyrazolyl ring.

In some embodiments, Z is trifluoromethanesulfonate.

In some embodiments, the compound of formula (III) has the following formula:

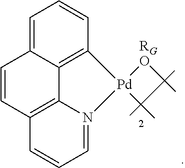

In some embodiments, the compound of formula (III) has the following formula:

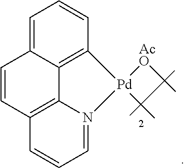

In some embodiments, the compound of formula (IV) has the following formula:

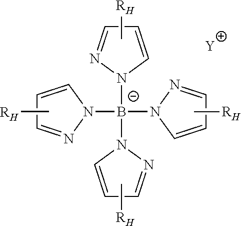

wherein each R_{H} is independently selected from hydrogen, alkyl, alkoxy, aryl or heteoraryl. In some embodiments, each R_{H} is independently a hydrogen. In some embodiments, each R_{H} is halogen (e.g., 4-chloro).

In some embodiments, the compound of formula (IV) has the following formula:

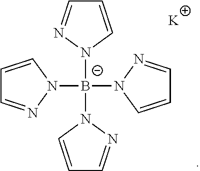 [SMILES: c1cnn([B-](n2cccn2)(n2cccn2)n2cccn2)c1 ; [K+]]

In some embodiments, the compound of formula (IV) has the following formula:

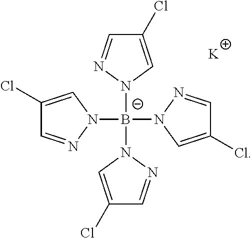 [SMILES: Clc1cnn([B-](n2cc(Cl)cn2)(n2cc(Cl)cn2)n2cc(Cl)cn2)c1 ; [K+]]

In some embodiments, the compound of formula (V) has the following formula:

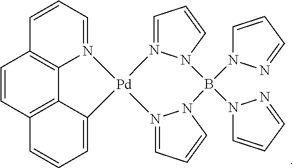

In some embodiments, the compound of formula (V) has the following formula:

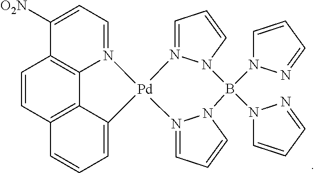

In some embodiments, the compound of formula (V) has the following formula:

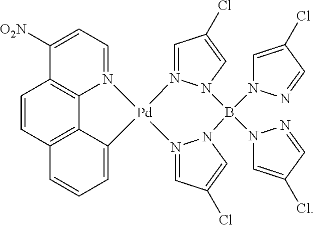

In some embodiments, the compound of formula (V) has the following formula:

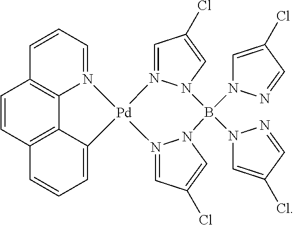

In some embodiments, the compound of formula (VI) has the following formula:

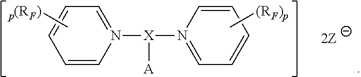

In some embodiments, the compound of formula (VI) has the following formula:

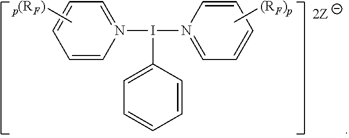

In some embodiments, the compound of formula (VI) is selected from

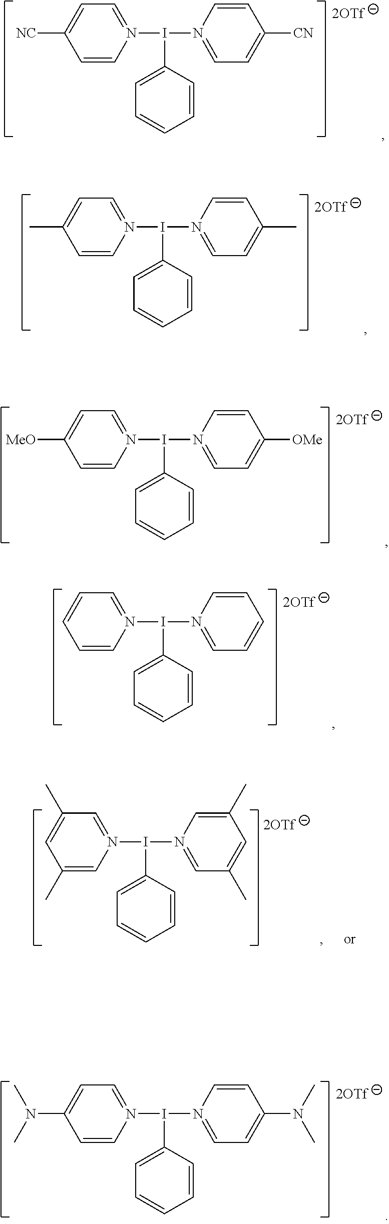

In some embodiments, the compound of formula (VI) is

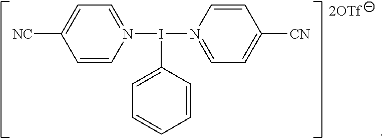

In some embodiments, the palladium complex of formula (I) is selected from one of the following:

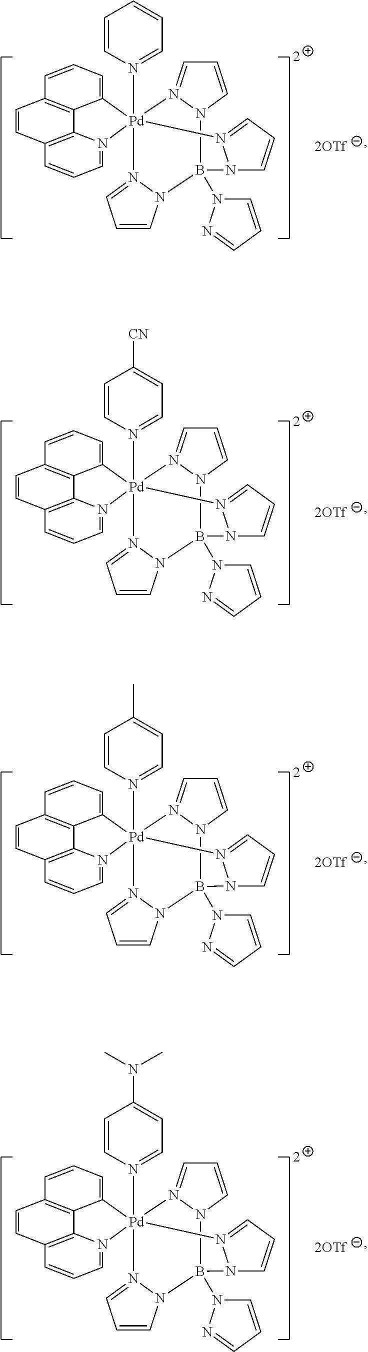

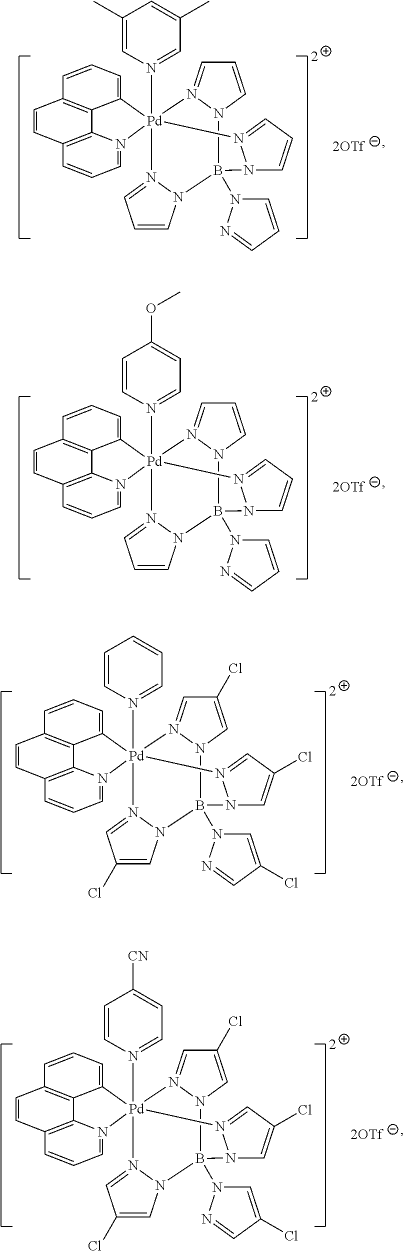

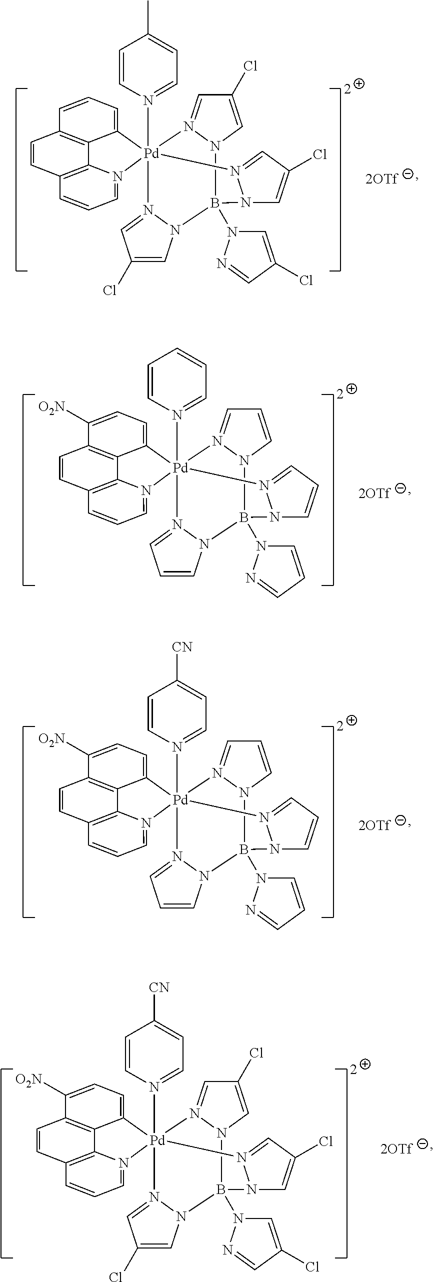

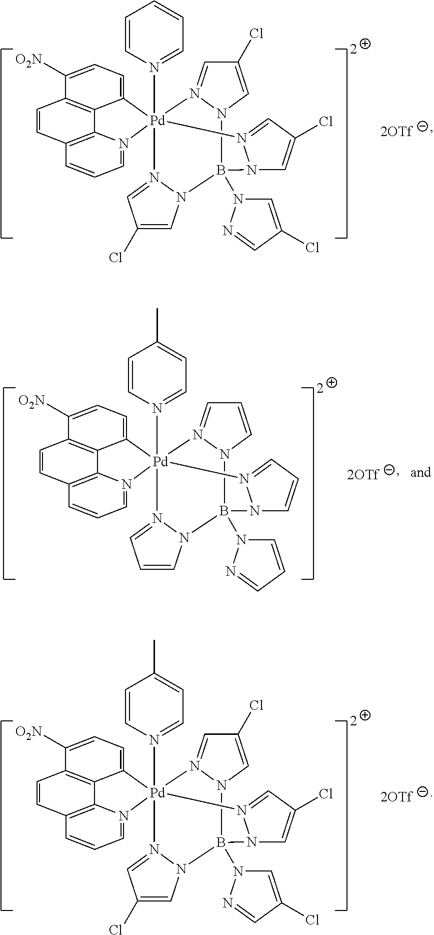

In some embodiments, the palladium complex of formula (I) has the following formula:

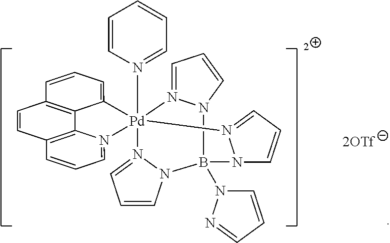

In some embodiments, the palladium complex of formula (I) has the following formula:

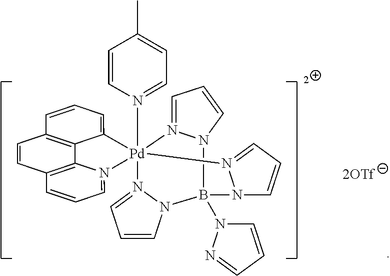

In some embodiments, the palladium complex of formula (I) is mixed with F— to produce a palladium (IV) complex and subsequently said palladium (IV) complex is reacted with an organic compound under conditions sufficient to fluorinate the compound, thereby providing a fluorinated organic compound.

In some embodiments, F— comes from a source of F—. In some embodiments, a source of F— is cesium fluoride (CsF) or potassium fluoride (KF).

In some embodiments, F⁻ comprises ¹⁸F⁻. In some embodiments, F⁻ comprises ¹⁹F⁻.

In some embodiments, the electrophilic fluorinating reagent comprises ¹⁸F. In some embodiments, the electrophilic fluorinating reagent comprises ¹⁹F.

In some embodiments, the method further comprises a solvent. In some embodiments, the solvent is a polar aprotic solvent. In some embodiments, the solvent is acetone. In some embodiments, the solvent is methylene chloride. In some embodiments, the solvent is dichloroethane. In some embodiments, the solvent is tetrahydrofuran. In some embodiments, the solvent is acetonitrile. In some embodiments, the solvent is 1,2-dichloroethane.

In some embodiments, the method further comprises an inert atmosphere.

In some embodiments, the reaction is performed under anhydrous conditions.

In some embodiments, the reaction comprises a source of energy. In some embodiments, the reaction comprises heat.

In some embodiments, the fluorinated organic compound comprises an aryl group. In some embodiments, the fluorinated organic compound is 3,4-dihydroxy-6-fluoro-DL-phenylalanine monohydrate (F-DOPA). In some embodiments, the fluorinated organic compound is a fluoroestrone. In some embodiments, the fluorinated organic compound is 1-(benzyloxy)-3-fluorobenzene. In some embodiments, the fluorinated organic compound is 2-fluoro-3,4-dihydronaphthalen-1(2H)-one.

In some embodiments, the fluorinated organic compound has the following structure:

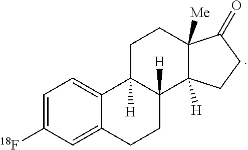

In another aspect, the present invention is directed to a method of making a palladium complex of formula (IX), the method comprising treating a palladium complex of formula (I) with an anionic reagent to produce a palladium complex of formula (IX).

In some embodiments, the palladium complex of formula (I) is:

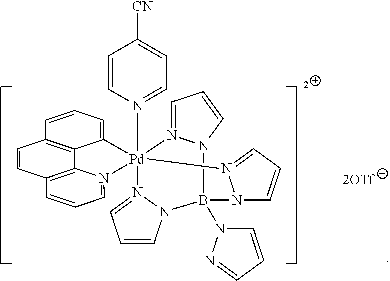

In some embodiments, the palladium complex of formula (I) is:

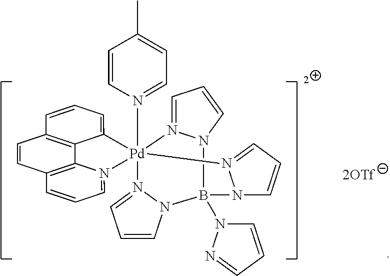

In some embodiments, the anionic reagent is NaNO₃.

In some embodiments, the composition further comprises a solvent. In some embodiments, the solvent is a polar aprotic solvent. In some embodiments, the solvent is a nonpolar solvent. In some embodiments, the solvent is acetonitrile.

In some embodiments, the composition further comprises a phase transfer catalyst. In some embodiments, the phase transfer catalyst is a crown ether. In some embodiments, the crown ether is 18-crown-6.

In some embodiments, the method is carried out under an inert atmosphere.

In some embodiments, the method is performed under anhydrous conditions.

In some embodiments, the method is carried out in the presence of a source of energy. In some embodiments, the source of energy is heat.

In another aspect, the present invention is directed to a method of storing a palladium complex of formula (I), the method comprising maintaining the palladium complex in a sealed container for at least 12 hours.

In some embodiments, the sealed container is a vial. In some embodiments, the sealed container is an ampule.

In some embodiments, the sealed container is substantially free of dioxygen. In some embodiments, the sealed container contains an inert gas.

In another aspect, the present invention is directed to a composition comprising a palladium complex of formula (I).

In some embodiments, the composition further comprises a solvent. In some embodiments, the solvent is a polar aprotic solvent. In some embodiments, the solvent is acetone. In some embodiments, the solvent is methylene chloride. In some embodiments, the solvent is dichloroethane. In some embodiments, the solvent is tetrahydrofuran.

In another aspect, the present invention is directed to a palladium complex of formula (II).

In some embodiments, the composition further comprises a solvent. In some embodiments, the solvent is a polar aprotic solvent. In some embodiments, the solvent is acetone. In some embodiments, the solvent is methylene chloride. In some embodiments, the solvent is dichloroethane. In some embodiments, the solvent is tetrahydrofuran.

In another aspect, the present invention is directed to a palladium complex of formula (I).

In some embodiments, the reaction mixture further comprises an organic compound.

In some embodiments, the reaction mixture further comprises a solvent. In some embodiments, the solvent is a polar aprotic solvent. In some embodiments, the solvent is acetone. In some embodiments, the solvent is methylene chloride. In some embodiments, the solvent is dichloroethane. In some embodiments, the solvent is tetrahydrofuran.

In some embodiments, the reaction mixture further comprises an inert atmosphere.

In another aspect, the present invention is directed to a reaction mixture comprising a palladium complex of formula (II).

In some embodiments, the reaction mixture further comprises an organic compound.

In some embodiments, the reaction mixture further comprises a solvent. In some embodiments, the solvent is a polar aprotic solvent. In some embodiments, the solvent is acetone. In some embodiments, the solvent is methylene chloride. In some embodiments, the solvent is dichloroethane. In some embodiments, the solvent is tetrahydrofuran.

In some embodiments, further comprising an inert atmosphere.

In another aspect, the present invention is directed to a kit comprising a palladium complex of formula (I) and a container.

In some embodiments, the container is a vial. In some embodiments, the container is a sealed ampule.

In some embodiments, the container is substantially free of dioxygen.

In some embodiments, the container contains an inert gas.

In some embodiments, the kit further comprises instructions for use of the palladium complex.

In some embodiments, the kit further comprises a reagent. In some embodiments, the kit further comprises a substrate. In some embodiments, the substrate is an organic compound.

This application refers to various issued patents, published patent applications, journal articles, and other publications, all of which are incorporated herein by reference.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

Certain compounds of the present invention can comprise one or more asymmetric centers, and thus can exist in various isomeric forms, e.g., stereoisomers and/or diastereomers. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain embodiments, mixtures of stereoisomers or diastereomers are provided.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds.

Where a particular enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); Wilen, Tables of Resolving Agents and Optical Resolutions p. 268 (E. L. Eliel, Ed., Univ. of Notre Dame Press, Notre Dame, Ind. 1972).

As used herein, a “bond” refers to a single bond.

The terms “halo” and “halogen” as used herein refer to an atom selected from fluorine (fluoro, —F), chlorine (chloro, —Cl), bromine (bromo, —Br), and iodine (iodo, —I).

The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents).

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-10 carbon atoms. In certain embodiments, aliphatic groups contain 1-8 carbon atoms, 1-7 carbon atoms, 1-6 carbon atoms, 1-5 carbon atoms, 1-4 carbon atoms, 1-3 carbon atoms, or 1-2 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “unsaturated”, as used herein, means that a moiety has one or more double or triple bonds.

The terms “carbocyclyl” and “carbocyclic” refer to saturated or partially unsaturated cyclic aliphatic monocyclic or bicyclic ring systems, as described herein, having from 3 to 10 members, wherein the aliphatic ring system is optionally substituted as defined above and described herein. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In certain embodiments, the cycloalkyl has 3-6 carbons. The terms “cycloaliphatic”, “carbocycle” or “carbocyclic” also include aliphatic rings that are fused to one or more aromatic or nonaromatic rings, such as decahydronaphthyl or tetrahydronaphthyl, where the radical or point of attachment is on the aliphatic ring.

The term “alkyl,” as used herein, refers to saturated, straight- or branched-chain hydrocarbon radicals derived from an aliphatic moiety containing between one and six carbon atoms by removal of a single hydrogen atom. In certain embodiments, the alkyl group employed in the invention contains 1-10 carbon atoms. In certain embodiments, the alkyl group employed contains 1-8 carbon atoms, 1-7 carbon atoms, 1-6 carbon atoms, 1-5 carbon atoms, 1-4 carbon atoms, 1-3 carbon atoms, or 1-2 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

The term “alkenyl,” as used herein, denotes a monovalent group derived from a straight- or branched-chain aliphatic moiety having at least one carbon-carbon double bond by the removal of a single hydrogen atom. In certain embodiments, the alkenyl group employed in the invention contains 2-10 carbon atoms. In certain embodiments, the alkenyl group employed in the invention contains 2-8 carbon atoms, 2-7 carbon atoms, 2-6 carbon atoms, 2-5 carbon atoms, 2-4 carbon atoms, 2-3 carbon atoms or 2 carbon atoms. Alkenyl groups include, for example, ethenyl, propenyl, butenyl, 1-methyl-2-buten-1-yl, and the like.

The term “alkynyl,” as used herein, refers to a monovalent group derived from a straight- or branched-chain aliphatic moiety having at least one carbon-carbon triple bond by the removal of a single hydrogen atom. In certain embodiments, the alkynyl group employed in the invention contains 2-10 carbon atoms. In certain embodiments, the alkynyl group employed in the invention contains 2-8 carbon atoms, 2-7 carbon atoms, 2-6 carbon atoms, 2-5 carbon atoms, 2-4 carbon atoms, 2-3 carbon atoms or 2 carbon atoms. Representative alkynyl groups include, but are not limited to, ethynyl, 2-propynyl (propargyl), 1-propynyl, and the like.

The term “aryl” refers to monocyclic, bicyclic or tricyclic aromatic ring system having a total of five to 14 ring members, wherein at least one ring in the system is aromatic and wherein each ring in the system contains three to seven ring members. The term “aryl” may be used interchangeably with the term “aryl ring”. In certain embodiments of the present invention, “aryl” refers to a monocyclic or polycyclic aromatic ring system which includes, but is not limited to, phenyl, biphenyl, naphthyl, anthracyl, phenanthrenyl, phenalenyl, and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenantriidinyl, or tetrahydronaphthyl, and the like.

The term “heteroaryl” refers to a monocyclic, bicyclic or tricyclic aromatic ring system having 5 to 14 ring atoms, wherein the ring atoms include carbon atoms and from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring” any of which terms include rings that are optionally substituted.

As used herein, the terms “heterocyclyl” and “heterocyclic ring” are used interchangeably and refer to a monocyclic, bicyclic or tricyclic nonaromatic ring sytem that is either saturated or partially unsaturated, and having, in addition to carbon atoms, one to five heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, and “heterocyclyl ring”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R′; —(CH₂)₀₋₄OR′; —O—(CH₂)₀₋₄C(O)OR′; —(CH₂)₀₋₄CH(OR′)₂; —(CH₂)₀₋₄SR′; —(CH₂)₀₋₄Ph, which may be substituted with R′; —(CH₂)₀₋₄O(CH₂)₀₋₁Ph which may be substituted with R′; —CH═CHPh, which may be substituted with R′; —NO₂; —CN; —N₃; —(CH₂)₀₋₄N(R′)₂; —(CH₂)₀₋₄N(R′)C(O)R′; —N(R′)C(S)R′; —(CH₂)₀₋₄N(R′)C(O)NR′₂; —N(R′)C(S)NR′₂; —(CH₂)₀₋₄N(R′)C(O)OR′; —N(R′)N(R′)C(O)R′; —N(R′)N(R′)C(O)NR′₂; —N(R′)N(R′)C(O)OR′; —(CH₂)₀₋₄C(O)R^{o}; —C(S)R^{o}; —(CH₂)₀₋₄C(O)OR′; —(CH₂)₀₋₄C(O)SR′; —(CH₂)₀₋₄C(O)OSiR′₃; —(CH₂)₀₋₄OC(O)R′; —OC(O)(CH₂)₀₋₄SR—, SC(S)SR′; —(CH₂)₀₋₄SC(O)R′; —(CH₂)₀₋₄C(O)NR′₂; —C(S)NR′₂; —C(S)SR′; —SC(S)SR′, —(CH₂)₀₋₄C(O)NR′₂; —C(O)N(OR′)R′; —C(O)C(O)R′; —C(O)CH₂C(O)R′; —C(NOR′)R′; —(CH₂)₀₋₄SSR′; —(CH₂)₀₋₄S(O)₂R′; —(CH₂)₀₋₄S(O)₂OR′; —(CH₂)₀₋₄OS(O)₂R′; —S(O)₂NR′₂; —(CH₂)₀₋₄S(O)R′; —N(R′)S(O)₂NR′₂; —N(R′)S(O)₂R′; —N(OR′)R′; —C(NH)NR′₂; —P(O)₂R′; —P(O)R′₂; —OP(O)R′₂; —OP(O)(OR′)₂; SiR′₃; —(C₁₋₄ straight or branched alkylene)O—N(R′)₂; or —(C₁₋₄ straight or branched alkylene)C(O)O—N(R′)₂, wherein each R′ may be substituted as defined below and is independently hydrogen, C₁₋₆ aliphatic, —CH₂Ph, —O(CH₂)₀₋₁Ph, or a 5-6-membered saturated, partially unsaturated, or aryl ring having 0 heteroatoms independently selected from nitrogen, oxygen, or sulfur, or, notwithstanding the definition above, two independent occurrences of R′, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R′ (or the ring formed by taking two independent occurrences of R′ together with their intervening atoms), are independently halogen, —(CH₂)₀₋₂R″, -(haloR″), —(CH₂)₀₋₂OH, —(CH₂)₀₋₂OR″, —(CH₂)₀₋₂CH(OR″)₂; —O(haloR″), —CN, —N₃, —(CH₂)₀₋₂C(O)R″, —(CH₂)₀₋₂C(O)OH, —(CH₂)₀₋₂C(O)OR″, —(CH₂)₀₋₂SR″, —(CH₂)₀₋₂SH, —(CH₂)₀₋₂NH₂, —(CH₂)₀₋₂NHR″, —(CH₂)₀₋₂NR″₂, —NO₂, —SiR″₃, —OSiR″₃, —C(O)SR″, —(C₁₋₄ straight or branched alkylene)C(O)OR″, or —SSR″ wherein each R″ is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R′ include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R″, -(haloR″), —OH, —OR″, —O(haloR″), —CN, —C(O)OH, —C(O)OR″, —NH₂, —NHR″, —NR″₂, or —NO₂, wherein each R″ is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R″, -(haloR″), —OH, —OR″, —O(haloR″), —CN, —C(O)OH, —C(O)OR″, —NH₂, —NHR″, —NR″₂, or —NO₂, wherein each R″ is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

An “suitable amino-protecting group,” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-14/1 biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl) 6 chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethyl amino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

A “suitable hydroxyl protecting group” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4 dimethoxybenzyl carbonate, alkyl o-nitrobenzyl carbonate, alkyl p-nitrobenzyl carbonate, alkyl S-benzyl thiocarbonate, 4 ethoxy-1-napththyl carbonate, methyl dithiocarbonate, 2-iodobenzoate, 4-azidobutyrate, 4-nitro 4 methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro 1 methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, a-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS),1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

A “pharmaceutically acceptable form thereof” includes any pharmaceutically acceptable salts, isomers, and/or polymorphs of a palladium complex, or any pharmaceutically acceptable salts, prodrugs and/or isomers of an organic compound, as described below and herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As used herein, the term “prodrug” refers to a derivative of a parent compound that requires transformation within the body in order to release the parent compound. In certain cases, a prodrug has improved physical and/or delivery properties over the parent compound. Prodrugs are typically designed to enhance pharmaceutically and/or pharmacokinetically based properties associated with the parent compound. The advantage of a prodrug can lie in its physical properties, such as enhanced water solubility for parenteral administration at physiological pH compared to the parent compound, or it enhances absorption from the digestive tract, or it may enhance drug stability for long-term storage. The compounds of the invention readily undergo dehydration to form oligomeric anhydrides by dehydration of the boronic acid moiety to form dimers, trimers, and tetramers, and mixtures thereof. These oligomeric species hydrolyze under physiological conditions to reform the boronic acid. As such, the oligomeric anhydrides are contemplated as a “prodrug” of the compounds of the present invention, and may be used in the treatment of disorder and/or conditions a wherein the inhibition of FAAH provides a therapeutic effect.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, an isomer/enantiomer may, in some embodiments, be provided substantially free of the corresponding enantiomer, and may also be referred to as “optically enriched.” “Optically-enriched,” as used herein, means that the compound is made up of a significantly greater proportion of one enantiomer. In certain embodiments the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

As used herein, “polymorph” refers to a crystalline complex or compound existing in more than one crystalline form/structure. When polymorphism exists as a result of difference in crystal packing it is called packing polymorphism. Polymorphism can also result from the existence of different conformers of the same molecule in conformational polymorphism. In pseudopolymorphism the different crystal types are the result of hydration or solvation.

As used herein “coordinated” means the organic compound is associated with the palladium atom.

### DETAILED DESCRIPTION OF CERTAIN EMBODIMENTS OF THE INVENTION

The present invention provides novel high-valent palladium complexes. The complexes have terminal fluoride ligands, and the palladium center has an oxidation state greater than +2. In certain embodiments, the palladium center has an oxidation state of +4. The ligands surrounding the complex stabilize the octahedral coordination sphere, thus disfavoring reductive elimination and other reductive pathways. These complexes are useful in transferring an eletrophilic fluorine to an organic compound. In particular, the inventive complexes are useful in labelling a compound with ¹⁸F for positron emission tomography (PET). Also described herein are compositions, reaction mixtures and kits comprising the palladium complexes. Also described herein are methods for fluorinating organic compounds using a palladium complex, e.g., a palladium complex described herein.

### High-Valent Palladium Complexes

The present invention provides novel high-valent palladium complexes. In certain embodiments, the complex is a Pd (IV) complex. Typically, the complex comprises one or more bidentate or tridentate ligands. In certain embodiments, the inventive high-valent palladium complex is of the formula:

In one aspect, the present invention is directed to a palladium complex of formula (VII):

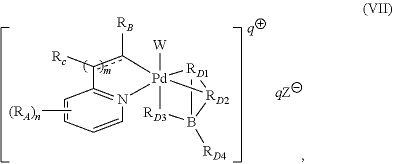

wherein:

the dashed line represents the presence or absence of a bond;

Pd is in the oxidation state +IV;

W is Br, hydroxyl, alkoxy, aryloxy, —NO₃, nitro, —N₃, ClO₄, PO₄, SO₄, —OSO₂-aryl, heteroaryl or heterocyclyl, each of which is substituted with p occurrences of R_{F};

n is 0, 1, 2, 3 or 4;

m is 0, 1, 2 or 3;

p is 0, 1, 2 or 3;

q is 1 or 2;

each occurrence of R_{A} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; wherein two R_{A} may be taken together with the atoms to which they are attached to form a substituted or unsubstituted carbocyclic, heterocyclic, aryl or heteroaryl ring;

each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R_{C} is independently hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; wherein R_{C} and R_{B} may be taken together with the atoms to which they are attached to form a substituted or unsubstituted heterocyclic or heteroaryl ring; and wherein R_{C} and R_{A} may be taken together with the atoms to which they are attached to form a substituted or unsubstituted carbocyclic, heterocyclic, aryl or heteroaryl ring;

R_{D1}, R_{D2}, R_{D3}, and R_{D4} are each independently cyclic or acyclic, branched or unbranched aliphatic; cyclic or acyclic, branched or unbranched heteroaliphatic; branched or unbranched aryl; branched or unbranched heteroaryl; each of which is substituted with 0-3 occurrences of R_{H};

each occurrence of R_{H} is independently hydrogen, halogen, alkyl, alkoxy, aryl or heteroaryl;

each occurrence of R_{F} is independently halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an aryl moiety; a heteroaryl moiety; alkoxy; aryloxy; alkylthio; arylthio; amino, alkylamino, dialkylamino, heteroaryloxy; or heteroarylthio moiety; and

Z⁻ is an anion.

In certain embodiments, the inventive high-valent palladium complex is of the formula:

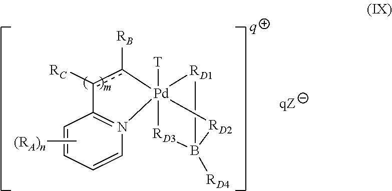

wherein:

the dashed line represents the presence or absence of a bond;

Pd is in the oxidation state +IV;

T is Br, hydroxyl, aryloxy, —NO₃, nitro, —N₃, ClO₄, PO₄, SO₄, or —O—SO₂-aryl; and

n, m, q, R_{A}, R_{B}, R_{C}, R_{D1}, R_{D2}, R_{D3}, R_{D4}, R_{F}, R_{H}, R″ and Z are as defined in formula (VII).

In certain embodiments, the inventive high-valent palladium complex is of the formula:

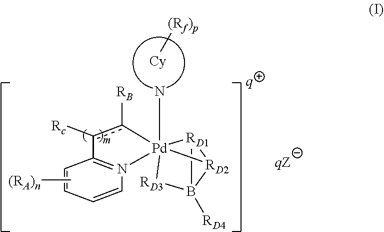

wherein:

the dashed line represents the presence or absence of a bond;

Pd is in the oxidation state +IV;

Cy taken together with the nitrogen atom to which it is attached forms a heterocyclyl or heteroaryl ring;

n, m, p, q, R_{A}, R_{B}, R_{C}, R_{D1}, R_{D2}, R_{D3}, R_{D4}, R_{F}, R_{H}, R″ and Z are as defined in formula (VII).

In some embodiments, the palladium complex has the following formula:

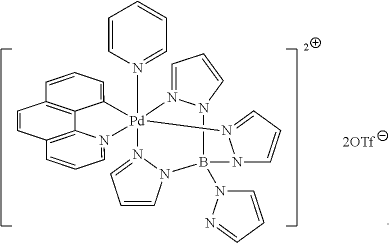

In some embodiments, the palladium complex has the following formula:

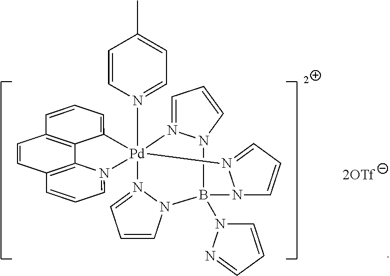

The counteranion Z⁻ may be any suitable anion. In certain embodiments, the counteranion has a charge of −1. In certain embodiments, the counteranion has a charge of −2. In certain embodiments, the counteranion has a charge of −3. The counteranion may be an organic or inorganic anion. In certain embodiments, the counteranion is an inorganic anion such as phosphate, hexafluorophosphate, hexafluoroantimonate, sulfate, perchlorate, azide, a halide such as fluoride, chloride, bromide or iodide, etc. In other embodiments, the counteranion is an organic anion such as a carboxylate (e.g., acetate), sulfonate, phosphonate, borate, etc. In certain embodiments, the counteranion is trifluoromethanesulfonate (triflate). In certain embodiments, the counteranion is tosylate. In certain embodiments, the counteranion is mesylate. In certain embodiments, the counteranion is hexafluorophosphate. In certain embodiments, the counteranion is tetraphenylborate. In certain embodiments, the counteranion is tetrafluoroborate. In certain embodiments, the counteranion tetrakis(pentafluorophenyl)borate. In certain embodiments, the counteranion is hexafluoroanimonate. In certain embodiments, the counterion is [B[3,5-(CF₃)₂C₆H₃]₄]⁻, commonly abbreviated as [BAr^{F}₄]⁻.

In some embodiments, Cy taken together with the nitrogen to which it is attached forms a heteroaryl ring. In some embodiments, Cy taken together with the nitrogen to which it is attached forms a pyridyl ring.

In some embodiments, R_{C} is hydrogen.

In some embodiments, n is 0. In some embodiments, n is 1. In some embodiments, m is 1. In some embodiments, p is 0.

In some embodiments, R_{A} and R_{C} taken together with the atoms to which they are attached form an aryl ring. In some embodiments, R_{A} and R_{C} taken together with the atoms to which they are attached form a phenyl ring. In some embodiments, R_{B} and R_{C} taken together with the atoms to which they are attached form an aryl ring. In some embodiments, R_{B} and R_{C} taken together to form a phenyl ring.

In some embodiments, the dashed line represents the absence of a bond. In some embodiments, the dashed line represents the presence of a bond.

In some embodiments, R_{D1}, R_{D2}, R_{D3} and R_{D4} are each a 5-membered heteroaryl ring. In some embodiments, R_{D1}, R_{D2}, R_{D3} and R_{D4} are each a pyrazolyl ring. In some embodiments, R_{D1}, R_{D2}, R_{D3} and R_{D4} are each an unsubstituted pyrazolyl ring.

In some embodiments, Z is trifluoromethanesulfonate.

In certain embodiments, a palladium complex described herein comprises a bidentate ligand of one of the formulae:

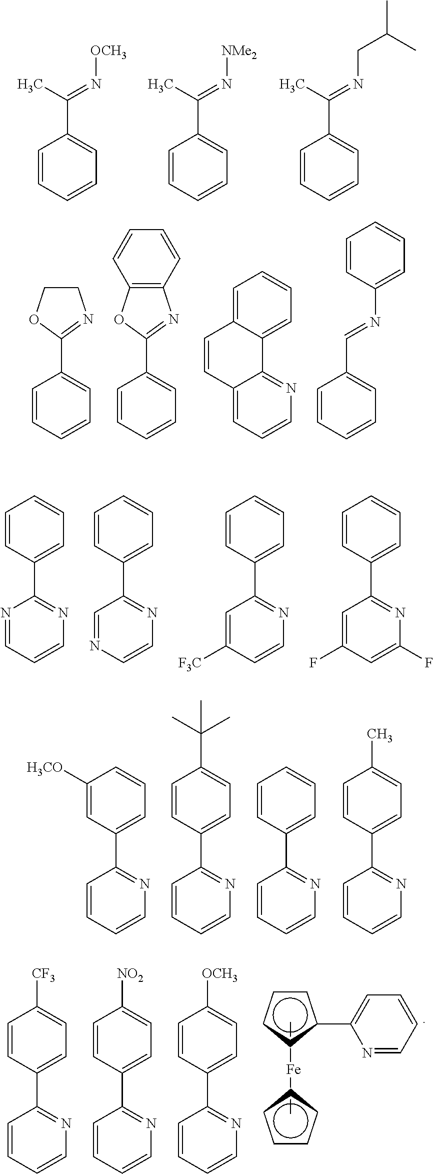

These ligands make a five-membered ring with the palladium atom with the nitrogen and a carbon coordinated to the central palladium.

In certain embodiments, the palladium complex is of the formula:

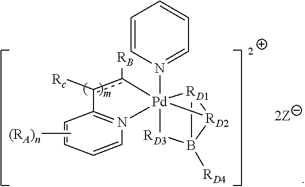

In certain embodiments, the palladium complex has the following formula:

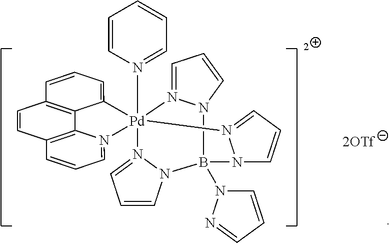

The present invention also provides novel high-valent palladium fluoride complexes. In certain embodiments, the complex is a Pd (IV) complex. Typically, the complex comprises one or more bidentate or tridentate ligands. In certain embodiments, the inventive high-valent palladium fluoride complex is of the formula:

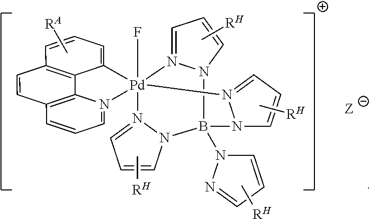

wherein:

R^{A} is as defined for formula (VII);

each R^{H} is independently selected from hydrogen, halogen, alkyl, alkoxy, aryl or heteoraryl;

F is comprises ¹⁸F or ¹⁹F; and

Z is an anion.

The present invention also provides novel high-valent palladium chloride complexes. In certain embodiments, the complex is a Pd (IV) complex. Typically, the complex comprises one or more bidentate or tridentate ligands. In certain embodiments, the inventive high-valent palladium chloride complex is of the formula:

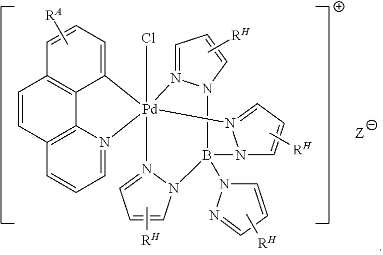

wherein:

R^{A}, R^{H} and Z are as defined in formula (II).

### Preparation of High-Valent Palladium Complexes

The inventive palladium complexes are typically prepared as described in the methods below. The method of making a palladium complex of formula (I) comprises treating a palladium complex of formula (III):

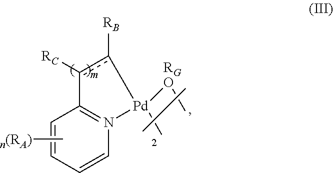

with a borate complex of formula (IV):

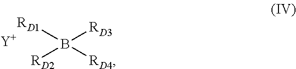

to provide a compound of formula (V):

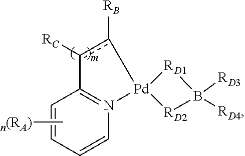

the method further comprising, treating a compound of formula (V) with a compound of formula (VI):

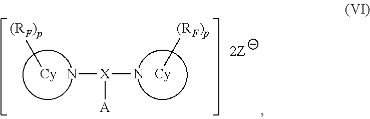

to provide a compound of formula (I), wherein
A is an aryl or heteroaryl group;
R_{G} is acyl;
Y⁺ is a cation;
X is a halogen; and
R_{A}, R_{E}, R_{C}, R_{D1}, R_{D2}, R_{D3}, R_{D4}, R_{F}, Z, R_{H}, R″, Cy, n, m and p are as defined for formula (I).

In some embodiments, the compound of formula (III) has the following formula:

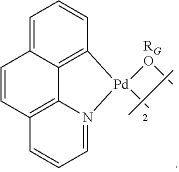

In some embodiments, the compound of formula (III) has the following formula:

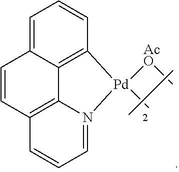

In some embodiments, the compound of formula (IV) has the following formula:

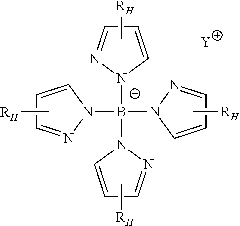

wherein each R_{H} is independently selected from hydrogen, alkyl, alkoxy, aryl or heteoraryl. In some embodiments, each R_{H} is independently a hydrogen.

In some embodiments, the compound of formula (IV) has the following formula:

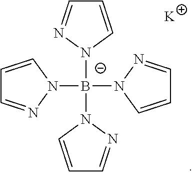

In some embodiments, the compound of formula (V) has the following formula:

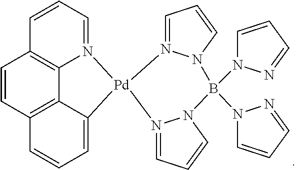

In some embodiments, the compound of formula (VI) has the following formula:

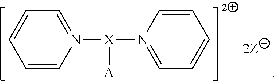

In some embodiments, the compound of formula (VI) has the following formula:

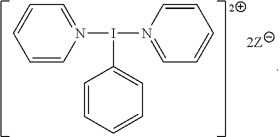

In some embodiments, the palladium complex of formula (I) has the following formula:

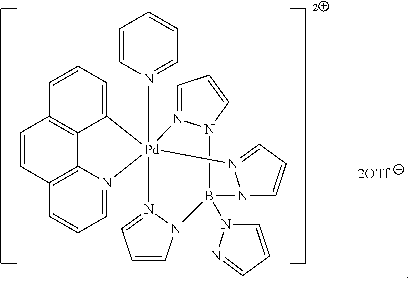

The inventive palladium complexes of formula (I) are typically prepared as described in the methods below. The method of making a palladium complex of formula (I) comprises treating a palladium complex of formula (III):

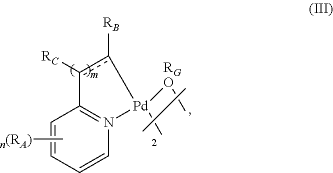

with a borate complex of formula (IV):

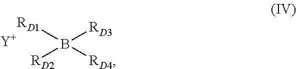

to provide a compound of formula (V):

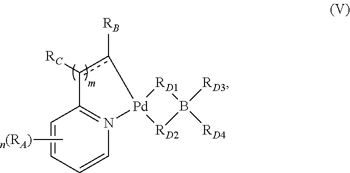

the method further comprising, treating a compound of formula (V) with a compound of formula (VI):

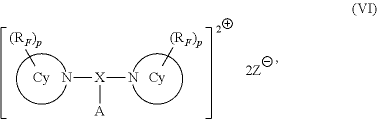

to provide a compound of formula (I), wherein
A is an aryl or heteroaryl group;
R_{G} is acyl;
Y⁺ is a cation;
X is a halogen; and
R_{A}, R_{B}, R_{C}, R_{D1}, R_{D2}, R_{D3}, R_{D4}, R_{F}, Z, Cy, n, m and p are as defined for formula (I).

The inventive palladium complexes of formula (IX) are typically prepared as described in the methods below. The method of making a palladium complex of formula (IX) comprises treating a palladium complex of formula (I) with a nucleophilic reagent to produce a palladium complex of formula (IX).

### Fluorinating Agents

As generally described above, the process for utilizing the high-valent palladium(IV) complexes described herein utilizes a fluorinating agent. In certain embodiments, the fluorinating agent is an electrophilic fluorinating agent. In certain embodiments, the fluorinating agent is commercially available. In certain embodiments, the electrophilic fluorinating agent is an inorganic fluorinating agent. Exemplary electrophilic fluorinating agents include, but are not limited to, N-fluoropyridinium triflate, trimethylpyridinium triflate, N-fluoro-2,4,6-trimethylpyridinium tetrafluoroborate, N-fluoro-2,6-dichloropyridinium tetrafluoroborate, N-fluoro-2,6-dichloropyridinium triflate, N-fluoropyridinium pyridine heptafluorodiborate, N-fluoropyridinium tetrafluoroborate, N-fluoropyridinium triflate, N-fluoroarylsulfonimide (e.g., N-fluorobenzenesulfonimide), N-chloromethyl-N′-fluorotriethylenediammonium bis(tetrafluoroborate) (Selectfluor®), and XeF₂. In certain embodiments, the fluorinating agent is Selectfluor®. In certain embodiments, the fluorinating agent is N-fluoropyridinium triflate. In certain embodiments, the fluorinating agent is N-fluoro-2,4,6-trimethylpyridinium triflate. In certain embodiments, the fluorinating agent is N-fluoro-2,4,6-trimethylpyridinium tetrafluoroborate. In certain embodiments, the fluorinating agent is N-fluoro-benzenesulfonimide. In certain embodiments, the fluorinating agent is xenon difluoride. In certain embodiments, the fluorinating agent is N-chloromethyl-N′-fluorotriethylenediammonium bis(tetrafluoroborate) (Selectfluor®).

In certain embodiments, the inventive high-valent palladium(IV) complexes may also utilize a nucleophilic fluoride reagent rather than electrophilic fluorinating reagent. In general, this may be accomplished by reacting high-valent palladium(IV) pyridine complex and subjecting the complex to halogen metathesis using KF as shown in the scheme below.

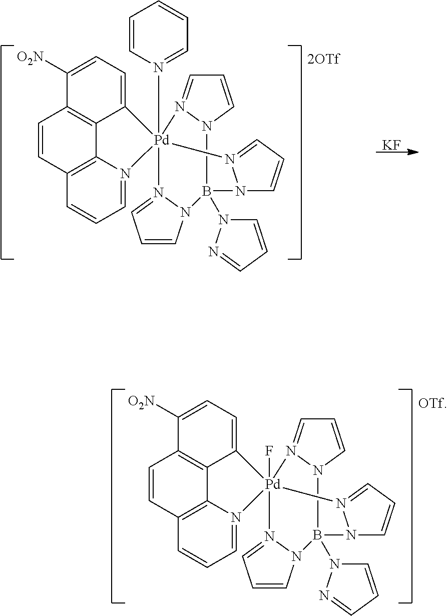

The fluorinating agent may be enriched with a particular isoptope of fluorine. In certain embodiments, the fluorinating agent is labeled with ¹⁹F (i.e., transfers an ¹⁹F fluorine substituent to the organic compound). In certain embodiments, reaction of the ¹⁹F fluorinating agent in the inventive process provides a fluorinated ¹⁹F-labeled organic compound.

In certain embodiments, the fluorinating agent is labeled with ¹⁸F (i.e., transfers an ¹⁸F fluorine substituent to the organic compound). In certain embodiments, reaction of the ¹⁸F fluorinating agent in the inventive process provides a fluorinated ¹⁸F-labeled organic compound.

However, in certain embodiments, the fluorinating agent is labeled with a mixture of ¹⁸F and ¹⁹F. In certain embodiments, reaction of the fluorinating agent with a mixture of ¹⁹F and ¹⁸F in the inventive process provides a mixture of fluorinated ¹⁹F-labeled organic compound and fluorinated ¹⁸F-labeled organic compound. In certain embodiments, the portion of each of ¹⁹F and ¹⁸F in the mixture is known. Any of the above fluorinated agents may be labeled with ¹⁹F or ¹⁸F.

For example, in certain embodiments, the fluorinating agent is ¹⁹F-labeled N-chloromethyl-N′-fluorotriethylenediammonium bis(tetrafluoroborate) (Selectfluor®) or ¹⁹F-labeled XeF₂. In certain embodiments, the fluorinating agent is ¹⁹F-labeled N-chloromethyl-N′-fluorotriethylenediammonium bis(tetrafluoroborate) (Selectfluor®). In certain embodiments, the fluorinating agent is ¹⁹F-labeled XeF₂.

In certain embodiments, the fluorinating agent is ¹⁸F-labeled XeF₂. In some embodiments, the fluorinating agent is ¹⁸F-labeled KF or CsF.

### Uses

The inventive high-valent palladium(IV) complexes are capable of converting a source of F— to an electrophilic fluorinating reagent. In some embodiments, the resulting electrophilic fluoride complexes are reactive toward nucleophiles such as palladium(II) complexes (e.g., palladium(II) aryl complexes), PPh₃, enamines, and enol silyl ethers. The electrophilic fluoride complexes resulting from the palladium (IV) complexes described herein are inventive complexes which may be useful in fluorination reactions by providing electrophilic fluorine. In particular, the high valent palladium complexes may be useful in conjunction with other transition metal reagents or catalysts for transfering the electrophilic F to an organic compound.

In certain embodiments, these high-valent palladium complexes or high-valent palladium fluoride complexes described herein are also used in conjunction with the palladium(II)-mediated fluorination reactions described in U.S. provisional patent application, U.S. Ser. No. 61/063,096, filed Jan. 31, 2008, and U.S. Ser. No. 61/050,446, filed May 5, 2008. Such reactions are particularly useful in preparing aryl fluorides. In some embodiments, the electrophilic fluorine can be ¹⁸F.

In certain embodiments, the high-valent palladium complexes or the resulting high-valent palladium fluoride complexes are reacted with enol silyl ethers under suitable conditions to yield alpha-fluorinated carbonyl compounds. In certain embodiments, the starting material is cyclohexanone enol trimethylsilyl ether. In certain embodiments, the high-valent palladium fluoride complexes are reacted with enamines under suitable conditions to yield fluorinated compounds.

### Organic Compounds

As generally described above, the invention provides a process for fluorinating an organic or organometallic compound using a high-valent palladium(IV) complex. In certain embodiments, the organic or organometallic compound has a particular substituent that is replaced with the fluoride from the complex.

The organic compound utilized in the inventive process includes, but is not limited to, small organic molecules and/or large organic molecules. A small organic molecule include any molecule having a molecular weight of less than 1000 g/mol, of less than 900 g/mol, of less than 800 g/mol, of less than 700 g/mol, of less than 600 g/mol, of less than 500 g/mol, of less than 400 g/mol, of less than 300 g/mol, of less than 200 g/mol or of less than 100 g/mol. A large organic molecule include any molecule of between 1000 g/mol to 5000 g/mol, of between 1000 g/mol to 4000 g/mol, of between 1000 g/mol to 3000 g/mol, of between 1000 g/mol to 2000 g/mol, or of between 1000 g/mol to 1500 g/mol. Organic compounds include, but are not limited to, aryl compounds, heteroaryl compounds, carbocyclic compounds, heterocyclic compounds, aliphatic compounds, heteroaliphatic compounds, as well as polymers, peptides, glycopeptides, and the like.

In certain embodiments, the organic compound is an optionally substituted aliphatic, optionally substituted heteroaliphatic, optionally substituted aryl, or optionally substituted heteroaryl compound. In certain embodiments, the organic compound is an aryl-containing compound.

In certain embodiments, an organic compound is a polymer.

In certain embodiments, an organic compound is a peptide.

In certain embodiments, an organic compound is biologically active.

For example, in certain embodiments, the organic compound is an agrochemical. In certain embodiments, the organic compound is an insecticide or a pheromone of insect origin.

In certain embodiments, the organic compound is pharmaceutical agent. For example, in certain embodiments, the pharmaceutical agent is an anti-emetic, anti-coagulant, anti-platelet, anti-arrhythmic, anti-herpertensive, anti-anginal, a lipid-modifying drug, sex hormone, anti-diabetic, antibiotic, anti-viral, anti-fungal, anti-cancer, immunostimulant, immunosuppressant, anti-inflammatory, anti-rheumatic, anesthetic, analgesic, anticonvulsant, hypnotic, anxiolytic, anti-psychotic, barbituate, antidepressant, sedative, anti-obesity, antihistime, anti-eleptic, anti-manic, opioid, anti-Parkinson, anti-Alzheimers, anti-dementia, an anti-substance dependance drug, cannabinoid, 5HT-3 antagonist, monoamine oxidase inhibitor (MAOI), selective serotonin reuptake inhibitor (SSRI), or stimulant. In certain embodiments, the pharmaceutical agent is a psychotropic agent. In certain embodiments, the pharmaceutical agent is any pharmaceutical agent approved by the United States Food and Drug Administration (FDA) for administration to a human (see, e.g., www.accessdata.fda.gov/scripts/cder/drugsatfda).

In certain embodiments, the pharmaceutical agent is an antibiotic. In certain embodiments, the pharmaceutical agent is a lipid modifying drug. In certain embodiments, the pharmaceutical agent is a CNS drug (i.e., drug acting on the Central Nervous System). CNS drugs include, but are not limited to, hypnotics, anxiolytics, antipsychotics, barbituates, antidepressants, antiobesity, antihistimes, antieleptics, antimanics, opioids, analgesics, anti-Parkinson, anti-Alzheimers, anti-dementia, anti-substance dependance drugs, cannabinoids, 5HT-3 antagonists, monoamine oxidase inhibitors (MAOIs), selective serotonin reuptake inhibitors (SSRIs) and stimulants. Exemplary pharmaceutical agents such as antibiotics, lipid modifying agents and CNS agents are provided in International Application Nos. PCT/US2010/020544; PCT/US2010/020540 and PCT/US2010/041561, each of which is incorporated by reference herein in its entirety.

In certain embodiments, the organic compound, after fluorination, is biologically active. In certain embodiments, the organic compound, prior to fluorinated, is also biologically active.

In certain embodiments, the process provides after fluorination of the organic compound a known biologically active fluorinated compound, such as a fluorinated agrochemical or fluorinated pharmaceutical agent.

For example, in certain embodiments, the process provides after fluorination of the organic compound the known fluorinated pharmaceutical agent L-DOPA:

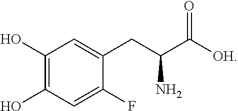 [SMILES: N[C@@H](Cc1cc(O)c(O)cc1F)C(=O)O]

In certain embodiments, the process provides after fluorination of the organic compound the compound fluoro-deoxy ESTRONE:

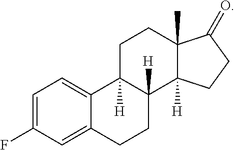

In certain embodiments, the process provides after fluorination of the organic compound the compound fluoro-deoxy ESTRADIOL:

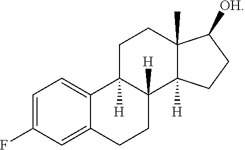

### Exemplary Reaction Conditions

Described herein are compositions comprising a palladium complex described herein, including a reaction mixture, e.g., a reaction mixture that is present during a method or process described herein. As defined generally herein, in certain embodiments, the process comprises mixing a substrate and a palladium(IV) complex described herein, under conditions sufficient to fluorinate the organic compound, to thereby provide a fluorinated organic compound.

In other embodiments, the process requires mixing a palladium(II) complex described herein with a fluorinating agent and a substrate, under conditions sufficient to fluorinate the substrate, thereby providing a fluorinated organic compound. In certain embodiments, the palladium(II) complex is combined with the fluorinating agent prior to addition of the substrate. In certain embodiments, this step results in formation of an intermediate palladium(IV) complex, which may or may not be isolated.

In certain embodiments, the palladium complex is bound to a solid support.

The substrate may be an organic compound comprising an enol silyl ether, or an organometallic compound such as a palladium(II) aryl complex or an arylsilver complex.

In certain embodiments, the method further comprises a solvent. In certain embodiments, the solvent is an organic solvent. In certain embodiments, the solvent is an aprotic solvent. Exemplary organic solvents include, but are not limited to, benzene, toluene, xylenes, methanol, ethanol, isopropanol, acetonitrile, acetone, ethyl acetate, ethyl ether, tetrahydrofuran, methylene chloride, dichloroethane and chloroform, or a mixture thereof. In certain embodiments, the solvent is acetonitrile. In certain embodiments, the solvent is methylene chloride. In certain embodiments, the solvent is tetrahydrofuran. In certain embodiments, the solvent is dichloroethane. In certain embodiments, the solvent is benzene.

In certain embodiments, the reaction further comprises heating. In certain embodiments, the reaction takes place under an inert atmosphere (e.g, an atmosphere of an inert gas such as nitrogen or argon). In certain embodiments, the reaction takes place under anhydrous conditions (e.g., conditions that are substantially free of water).

### Methods

Described herein are methods for fluorination of organic compounds. In certain embodiments, the fluorination reaction is regiospecific.

Introduction of fluorine into a certain position of bioactive compound such as a pharmaceutical agent and an agricultural chemical may remarkably reduce the toxicity of the compound. This is due to the mimic and blocking effect characterized by fluorine.

Organofluorine compounds are emerging as chemical specialties of significant and increasing commercial interest. A major driver has been the development of fluorine-containing bio-active molecules for use as medicinal and plant-protection agents. Other new applications involving organofluorine chemistry are in the synthesis of liquid crystals, surface active agents, specialty coatings, reactive dyes, and even olefin polymerization catalysts.

¹⁹F-fluorinated organic compounds may be useful for magnetic resonance imaging (MRI) technology. MRI is a primarily a medical imaging technique most commonly used in radiology to visualize the structure and function of the body. It provides detailed images of the body in any plane. MRI contrast agents are a group of contrast media used to improve the visibility of internal body structures in MRI. Contrast agents alter the relaxation times of tissues and body cavities where they are present, which depending on the image weighting can give a higher or lower signal. Fluorine-containing constrast agents may be especially useful due to the lack of fluorine chemistry in the human body. This could, for example provide a detailed view of acidic regions, such as those containing cancer cells. ¹⁹F-labeled MRI contrast agents may add chemical sensitivity to MRI and could be used to track disease progression without the need to take tissue or fluid samples.

¹⁹F-fluorinated organic compounds may also be useful as probes for nuclear magnetic resonance (NMR) spectroscopy. Fluorine has many advantages as a probe for NMR spectroscopy of biopolymers. ¹⁹F has a spin of one-half, and its high gyromagnetic ratio contributes to its high sensitivity (approximately 83% of the sensitivity of ¹H). It also facilitates long-range distance measurements through dipolar-dipolar coupling. Moreover, the near-nonexistence of fluorine atoms in biological systems enables ¹⁹F NMR studies without background signal interference. Furthermore, the chemical shift of ¹⁹F has been shown to be very sensitive to its environment.

¹⁸F-fluorinated organic compounds are particularly useful for positron-emission tomography (PET) imaging technology. PET is a noninvasive imaging technology that is currently used in the clinic to image cancers and neurological disorders at an early stage of illness. PET tracers are molecules which incorporate a PET-active nucleus and can therefore be visualized by their positron emission in the body. The fluorine isotope ¹⁸F is the most common nucleus for PET imaging because of its superior properties to other nuclei.

The ¹⁸F radioisotope has a half-life of 109 minutes. The short half-life dictates restrictions on chemical synthesis of PET tracers, because introduction of the fluorine atom has to take place at a very late stage of the synthesis to avoid the unproductive decay of ¹⁸F before it is injected into the body. Fluoride ion is the most common reagent to introduce ¹⁸F but the specific chemical properties of the fluoride ion currently limit the available pool of PET tracers. Due to the narrow functional group compatibility of the strongly basic fluoride ion, only a limited set of chemical reactions can be employed for fluorination, and hence the synthesis of PET tracers is limited to fairly simple molecules such as FDG. The field of PET imaging would benefit from the availability of a new method that is capable of introducing radiolabeled fluoride into structurally more complex organic molecules. An easy access to drug-based PET tracers would simplify determining the fate of such drugs in the body and thereby help to identify and understand their mode of action, bioavailability and time-dependent biodistribution. In certain embodiments, the PET tracer is represented by a compound of the following formula:

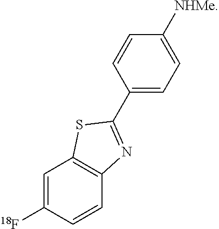 [SMILES: CNc1ccc(-c2nc3ccc([18F])cc3s2)cc1]

### Methods of Treatment

A fluorinated compound described herein, such as a fluorinated pharmaceutical agent, can be administered to cells in culture, e.g. in vitro or ex vivo, or to a subject, e.g., in vivo, to treat, prevent, and/or diagnose a variety of disorders, including those described herein below. In some embodiments, the fluorinated compound is made by a method described herein.

As used herein, the term “treat” or “treatment” is defined as the application or administration of a compound, alone or in combination with, a second compound to a subject, e.g., a patient, or application or administration of the compound to an isolated tissue or cell, e.g., cell line, from a subject, e.g., a patient, who has a disorder (e.g., a disorder as described herein), a symptom of a disorder, or a predisposition toward a disorder, with the purpose to cure, heal, alleviate, relieve, alter, remedy, ameliorate, improve or affect the disorder, one or more symptoms of the disorder or the predisposition toward the disorder (e.g., to prevent at least one symptom of the disorder or to delay onset of at least one symptom of the disorder).

As used herein, an amount of a compound effective to treat a disorder, or a “therapeutically effective amount” refers to an amount of the compound which is effective, upon single or multiple dose administration to a subject, in treating a cell, or in curing, alleviating, relieving or improving a subject with a disorder beyond that expected in the absence of such treatment.

As used herein, an amount of a compound effective to prevent a disorder, or a “a prophylactically effective amount” of the compound refers to an amount effective, upon single- or multiple-dose administration to the subject, in preventing or delaying the occurrence of the onset or recurrence of a disorder or a symptom of the disorder.

As used herein, the term “subject” is intended to include human and non-human animals. Exemplary human subjects include a human patient having a disorder, e.g., a disorder described herein or a normal subject. The term “non-human animals” of the invention includes all vertebrates, e.g., non-mammals (such as chickens, amphibians, reptiles) and mammals, such as non-human primates, domesticated and/or agriculturally useful animals, e.g., sheep, dog, cat, cow, pig, etc.

Described herein are compounds and compositions useful in the treatment of a disorder. In general, the compounds described herein are fluorinated derivatives of a pharmaceutical agent (e.g., a fluorinated estrone). Also envisioned herein are other compounds, wherein one or more fluorine moieties have been added to the pharmaceutical agent, e.g., replacing a hydrogen or functional group such as an —OH with a fluorine.

### Compositions and Routes of Administration

The compositions described herein may include a palladium complex described herein. In addition, a complex delineated herein may include the fluorinated compounds described herein, such as fluorinated pharmaceutical agents, as well as additional therapeutic agents if present, in amounts effective for achieving a modulation of disease or disease symptoms, including those described herein. In some embodiments, the fluorinated compound is made by a method described herein.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, infrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

### Kits

A compound described herein (e.g., a palladium complex described herein, a palladium fluoride complex described herein, an organic compound, a fluorinating agent, or a fluorinated compound, such as a fluorinated pharmaceutical agent) may be provided in a kit. The kit includes (a) a compound used in a method described herein, and, optionally (b) informational material. The informational material can be descriptive, instructional, marketing or other material that relates to the methods described herein and/or the use of the compounds for the methods described herein. In some embodiments, the palladium complex is bound to a solid support.

The informational material of the kits is not limited in its form. In one embodiment, the informational material can include information about production of the compound, molecular weight of the compound, concentration, date of expiration, batch or production site information, and so forth. In one embodiment, the informational material relates to methods for administering the compound.

In one embodiment, the informational material can include instructions to administer a compound described herein in a suitable manner to perform the methods described herein, e.g., in a suitable dose, dosage form, or mode of administration (e.g., a dose, dosage form, or mode of administration described herein). In another embodiment, the informational material can include instructions to administer a compound described herein to a suitable subject, e.g., a human, e.g., a human having or at risk for a disorder described herein.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to a compound described herein, the composition of the kit can include other ingredients, such as a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, and/or a second agent for treating a condition or disorder described herein. Alternatively, the other ingredients can be included in the kit, but in different compositions or containers than a compound described herein. In such embodiments, the kit can include instructions for admixing a compound described herein and the other ingredients, or for using a compound described herein together with the other ingredients.

In some embodiments, the components of the kit are stored under inert conditions (e.g., under Nitrogen or another inert gas such as Argon). In some embodiments, the components of the kit are stored under anhydrous conditions (e.g., with a desiccant). In some embodiments, the components are stored in a light blocking container such as an amber vial.

A compound described herein can be provided in any form, e.g., liquid, dried or lyophilized form. It is preferred that a compound described herein be substantially pure and/or sterile. When a compound described herein is provided in a liquid solution, the liquid solution preferably is an aqueous solution, with a sterile aqueous solution being preferred. When a compound described herein is provided as a dried form, reconstitution generally is by the addition of a suitable solvent. The solvent, e.g., sterile water or buffer, can optionally be provided in the kit.

The kit can include one or more containers for the composition containing a compound described herein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

### EXAMPLES

### General Methods

All air and moisture insensitive reactions were carried out under an ambient atmosphere, magnetically stirred, and monitored by thin layer chromatography (TLC) using EMD TLC plates pre-coated with 250 μm thickness silica gel 60 F254 plates and visualized by fluorescence quenching under UV light. Hash chromatography was performed on Dynamic Adsorbents Silica Gel 40-63 μm particle size using a forced flow of eluent at 0.3-0.5 bar pressure. (See W. C. Still et al.; J. Org. Chem. 43, 2925 (1978)) All air- and moisture-sensitive manipulations were performed using oven-dried glassware, including standard Schlenk and glovebox techniques under an atmosphere of nitrogen. Methylene chloride was purged with nitrogen, dried by passage through activated alumina, and stored over 3 Å molecular sieves. (See Pangborn, A. B et al.; Organometallics 15, 1518 (1996)). Benzene, benzene-d₆, diethyl ether, toluene, pentane, dioxane and THF were distilled from deep purple sodium benzophenone ketyl. Methylene chloride-d₂ was dried over CaH₂ and vacuum-distilled. Acetonitrile and d₃-acetonitrile were dried over P₂O₅ and vacuum-distilled. Pyridine was dried over CaH₂ and distilled. DMSO was distilled from sodium triphenylmethanide and stored over 3A sieves. Acetone was distilled over B₂O₃. (See W. S. Matthews et al. J. Am. Chem. Soc. 97, 7006 (1975)). MeOH was degassed and stored over over 3A sieves. Anhydrous DMF and dioxane bottles equipped with a SureSeal™ were purchased from Sigma Aldrich®. 18-Crown-6 was sublimed. KF was ground finely and dried at 200° C. under dynamic vacuum (10⁴ Torr) before use. Chloroform-d₁, D₂O, Pd(OAc)₂, AgOAc, and all other chemicals were used as received. All deutrated solvents were purchased from Cambridge Isotope Laboratories. Pd(OAc)₂, AgOAc, KBH₄, and 18-crown-6 were purchased from Strem Chemicals. Benzo[h]quinoline was purchased from TCI. (Diacetoxyiodo)benzene, potassium fluoride, 4-cyanopyridine, α-tetralone, pyrrolidine, p-toluenesulfonic acid, and p-methoxybenzenesulfonamide were purchased from Sigma-Aldrich®. Pyrazole, TMSOTf, and trifluoroacetic acid were purchased from Oakwood Products. Soda lime glass bottles were purchased from Qorpak®. NMR spectra were recorded on either a Varian Unity/Inova 600 spectrometer operating at 600 MHz for ¹H acquisitions, a Varian Unity/Inova 500 spectrometer operating at 500 MHz and 125 MHz for ¹H and ¹³C acquisitions, respectively, a Varian Mercury 400 spectrometer operating at 375 MHz and 101 MHz for ¹⁹F and ¹³C acquisitions, respectively, or a Varian Mercury 300 spectrometer operating at 100 MHz for ¹¹B acquisitions. Chemical shifts were referenced to the residual proton solvent peaks (¹H: CDCl₃, δ 7.26; C₆D₆, δ 7.16; CD₂Cl₂, δ 5.32; D₂O, δ 4.79; (CD₃)₂SO, δ 2.50; CD₃CN, δ 1.94), solvent ¹³C signals (CDCl₃, δ 77.16; C₆D₆, δ 128.06; CD₂Cl₂, δ 53.84; CD₃CN, δ 1.32, (CD₃)₂SO, δ 39.52), dissolved or external neat PhF (¹⁹F, δ −113.15 relative to CFCl₃) or dissolved 3-nitrofluorobenzene (−112.0 ppm). Signals were listed in ppm, and multiplicity identified as s=singlet, br=broad, d=doublet, t=triplet, q=quartet, quin=quintet, m=multiplet; coupling constants in Hz; integration. Concentration under reduced pressure was performed by rotary evaporation at 25-30° C. at appropriate pressure. Purified compounds were further dried under high vacuum (0.01-0.05 Torr). Yields refer to purified and spectroscopically pure compounds.

No-carrier-added [¹⁸F]fluoride was produced from water 97% enriched in ¹⁸O (Sigma-Aldrich®) by the nuclear reaction ¹⁸O (p,n)¹⁸F using a Siemens Eclipse HP cyclotron and a silver-bodied target at MGH Athinoula A. Martinos Center for Biomedical Imaging. The produced [¹⁸F]fluoride in water was transferred from the cyclotron target by helium push. Liquid chromatographic analysis (LC) was performed with Agilent 1100 series HPLCs connected to a Carol and Ramsey Associates Model 105-S radioactivity detector. An Agilent Eclipse XDB-C18, 5 μm, 4.6×150 mm HPLC column was used for analytical analysis and a Waters Bondapak™ C18, 10 μm, 125 Å, 7.6×300 mm HPLC was used for preparative HPLC. Analytical HPLC used the following mobile phases: 0.1% CF₃CO₂H in water (A) 0.1% CF₃CO₂H in acetonitrile (B). Program: 50% (B) for 2 minutes then a gradient 50-95% (B) over 8 minutes. Preparative HPLC used the following mobile phases: 0.1% CF₃CO₂H in water (A) 0.1% CF₃CO₂H in acetonitrile (B). Program: 40% (B). In the analysis of the ¹⁸F-labeled compounds, isotopically unmodified reference substances were used for identification. Radioactivity was measured in a Capintec, Inc. CRC-25PET ion chamber. Solvents and reagents for radiochemical experiments: Acetone (HPLC grade) was distilled over B₂O₃ and subsequently redistilled before use. Acetonitrile was distilled over P₂O₅. Water was obtained from a Millipore Milli-Q Integral Water Purification System. 18-crown-6 was sublimed. Potassium bicarbonate (≧99.99%) and JandaJel™-polypyridine (100-200 mesh, extent of labeling: ˜8.0 mmol/g loading, 1% cross-linked) were purchased from Sigma-Aldrich® and dried at 23° C. for 24 hours under dynamic vacuum (10⁻⁴ Torr) before use. Cotton was washed with acetone and water and dried at 150° C.

### Example 1

### Synthesis of Palladium(IV) Pyridine Complexes

### Benzo[h]quinolinyl palladium acetate dimer (1)

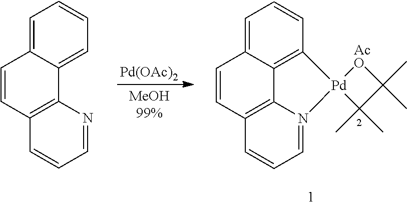

To benzo[h]quinoline (1.00 g, 5.58 mmol, 1.00 equiv) in MeOH (75 mL) at 23° C. was added Pd(OAc)₂ (1.25 g, 5.58 mmol, 1.00 equiv). After eight hours, the precipitate was isolated by filtration and washed sequentially with MeOH (50 mL) and Et₂O (50 mL). The solid was dissolved in CH₂Cl₂ (250 mL) and filtered through a plug of Celite. Solvent was removed in vacuo to afford 1.68 g of the title compound as a yellow solid (88% yield). NMR Spectroscopy: ¹H NMR (500 MHz, CDCl₃, 23° C., δ): 7.80 (dd, J=5.5 Hz, 1.5 Hz, 1H), 7.43 (dd, J=8.0 Hz, 1.5 Hz, 1H), 7.24-7.18 (m, 3H), 7.08 (dd, J=7.0 Hz, J=1.5 Hz, 1H), 6.97 (d, J=9.0 Hz, 1H), 6.46 (dd, J=7.5 Hz, 5.0 Hz, 1H), 2.38 (s, 3H). ¹³C NMR (125 MHz, CDCl₃, 23° C., δ): 182.5, 153.2, 148.9, 148.8, 140.0, 135.3, 132.4, 129.0, 127.9, 127.7, 125.0, 122.9, 122.1, 119.8, 25.2.

### Potassium tetra(1H-pyrazol-1-yl)borate

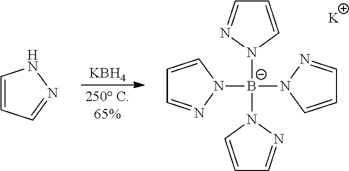 [SMILES: c1cn[nH]c1 ; "KBH4" ; c1cnn([B-](n2cccn2)(n2cccn2)n2cccn2)c1 ; [K+]]

As solids, KBH₄ (6.00 g, 0.11 mol, 1.00 equiv) and pyrazole (37.86 g, 0.56 mol, 5.00 equiv) were combined. This mixture was heated to 250° C. for 16 hours, after which time the melt was cooled to room temperature. The residue was triturated with Et₂O (300 mL) and isolated by filtration. Washing with additional Et₂O (2×100 mL) afforded 23.00 g of the title compound as a white solid (65% yield). Melting Point: 248-249° C. NMR Spectroscopy: ¹H NMR (600 MHz, D₂O, 23° C., δ): 7.49 (s, 4H), 7.19 (d, J=2.0 Hz, 4H), 6.14 (s, 4H). ¹³C NMR (125 MHz, D₂O, 23° C., δ): 138.85, 132.84, 102.42. ¹¹B NMR (100 MHz, D₂O, 23° C., δ): −1.30. Mass Spectrometry: LRMS-FIA (m/z): 279.1. These spectroscopic data corresponded to reported data. 1 Niedenzu, K.; Niedenzu, P. M. Inorg. Chem. 1984, 23, 3713-3716.

### Benzo[h]quinolinyl (tetrapyrazolylborate)palladium(II) (2)²

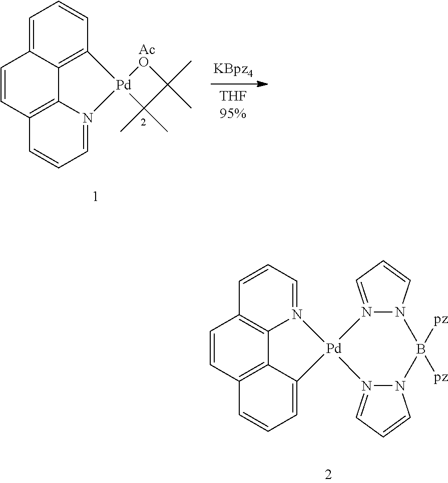

² Onishi, M.; Ohama, Y.; Sugimura, K.; Hiraki, K. Chem. Lett. 1976, 5, 955-958.

To benzo[h]quinolinyl palladium acetate dimer (1) (2.108 g, 3.07 mmol, 1.00 equiv) in 120 mL THF was added potassium tetra(1H-pyrazol-1-yl)borate (KBpz₄) (1.951 mg, 6.13 mmol, 2.00 equiv) in one portion at 23° C. The solution was stirred at room temperature for 12 hours at which time a white suspension is observed. Volatiles were removed in vacuo and the residue was dissolved in 200 mL CH₂Cl₂. The suspension was filtered through celite and volatiles were removed in vacuo. Trituration with Et₂O (100 mL) and filtration afforded 3.275 g of the title compound as a light yellow solid (95%). NMR Spectroscopy: ¹H NMR (400 MHz, CDCl₃, 23° C., δ): 8.53 (d, J=4.3 Hz, 1H), 8.25 (d, J=7.5 Hz, 1H), 7.97 (br s, 1H), 7.89 (br s, 1H), 7.77 (br s, 1H), 7.74 (d, J=8.6 Hz, 1H), 7.67 (br s, 1H), 7.61 (br s, 1H), 7.59 (d, J=8.5 Hz, 1H), 7.54 (d, J=9.6 Hz, 1H), 7.46-7.40 (m, 2H), 7.31 (d, J=6.4 Hz, 1H), 6.93 (br s, 1H), 6.44 (br s, 2H), 6.30 (br s, 1H), 6.00 (br s, 1H).

### Benzo[h]quinolinyl (tetrapyrazolylborate) palladium(IV) pyridine trifluorometahnesulfonate (3)

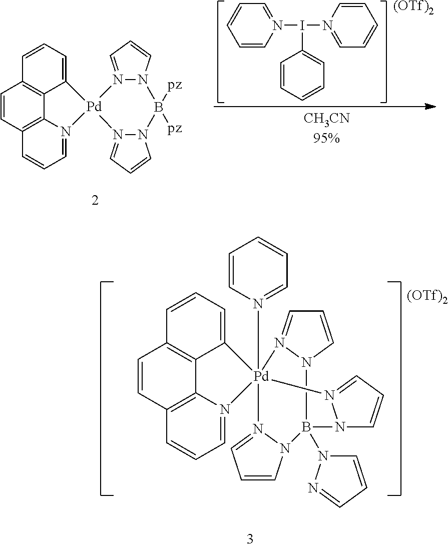

To benzo[h]quinolinyl (tetrapyrazolylborate)palladium(II) (2) (1.00 g, 1.774 mmol, 1.00 equiv) in CH₃CN (13 mL) at 23° C. was added bis(pyridinio-1)iodobenzene bis(trifluoroacetate)³ (1.195 g, 1.81 mmol, 1.02 equiv). After stirring for 20 min the reaction mixture was concentrated in vacuo. The resulting residue was triturated with THF (3×30 mL) and collected as a light brown solid. The solid was triturated with pentane (3×30 mL) and collected to afford 1.580 g of the title compound as a light brown solid (95%). NMR Spectroscopy: ¹H NMR (500 MHz, CD₃CN, 23° C., δ): 9.07 (d, J=7.5 Hz, 1H), 8.98 (d, J=2.1 Hz, 1H), 8.96 (d, J=6.4 Hz, 1H), 8.43 (dd, J=39.5 Hz, J=9.6 Hz, 2H), 8.37 (d, J=7.5 Hz, 1H), 8.24 (d, J=2.1 Hz, 2H), 8.11-7.94 (m, 5H), 7.84 (t, J=8.0 Hz, 1H), 7.74 (d, J=8.6 Hz, 1H), 7.51 (s, 1H), 7.50 (s, 1H), 7.39-7.36 (m, 3H), 6.85 (t, J=2.1 Hz, 1H), 6.80 (s, 2H), 6.20 (s, J=2.1 Hz, 1H), 6.09 (t, J=2.1 Hz, 1H). ³ Weiss, R.; Seubert, J. Angew. Chem., Int. Ed. 1994, 33, 891-893.

### 7-Nitrobenzo[h]quinolinyl palladium acetate dimer (1a)

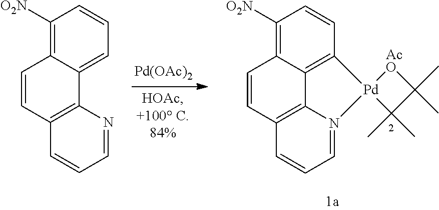

To 7-nitrobenzo[h]quinoline (1.00 g, 4.45 mmol, 1.00 equiv)⁴ in HOAc (30 mL) at 23° C. was added palladium acetate (1.00 g, 4.45 mmol, 1.00 equiv) and the reaction mixture was heated to 100° C. for 30 min. After being cooled to 23° C., the reaction mixture was concentrated in vacuo and triturated with Et2O (3×50 mL) to afford 1.45 g of the title compound as a red solid (84%). NMR Spectroscopy: ¹H NMR (500 MHz, CDCl₃, 23° C., δ): 8.22 (d, J=9.6 Hz, 1H), 8.08 (d, J=5.3 Hz, 1H), 7.91 (d, J=8.5 Hz, 1H), 7.83 (d, J=7.5 Hz, 1H), 7.36 (d, J=9.6 Hz, 1H), 7.13 (d, J=8.5 Hz, 1H), 6.94 (dd, J=8.5 Hz, J=5.3 Hz, 1H). ⁴ Furuya, T.; Benitez, D.; Tkatchouk, E.; Strom, A. E.; Tang, P.; Goddard III, W. A.; Ritter T. J. Am. Chem. Soc. 2010, 132, 3793-3807.

### 7-Nitrobenzo[h]quinolinyl (tetrapyrazolylborate)palladium(II) (2a)

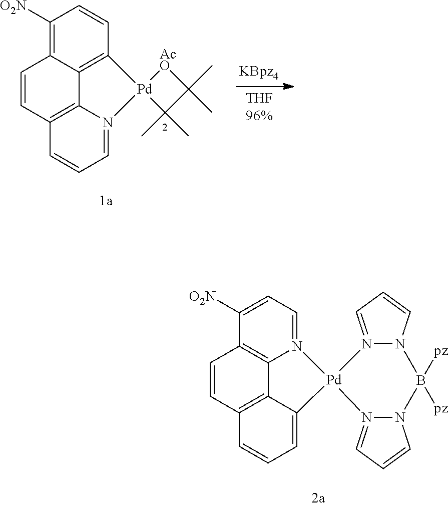

To 7-nitrobenzo[h]quinolinyl palladium acetate dimer (1) (1.372 g, 1.76 mmol, 1.00 equiv) in 100 mL THF was added potassium tetra(1H-pyrazol-1-yl)borate (KBpz₄) (1.123 mg, 3.53 mmol, 2.00 equiv) in one portion at 23° C. The solution was stirred at room temperature for 12 hours at which time a white suspension is observed. Volatiles were removed in vacuo and the residue was dissolved in 200 mL CH₂Cl₂. The suspension was filtered through celite and volatiles were removed in vacuo. Trituration with Et₂O (100 mL) and filtration afforded 2.056 g of the title compound as a yellow solid (96%). NMR Spectroscopy: ¹H NMR (500 MHz, CDCl₃, 23° C., δ): 8.56 (d, J=5.3 Hz, 1H), 8.30 (d, J=7.5 Hz, 1H), 7.97 (br s, 1H), 7.89 (br s, 1H), 7.80-7.29 (br m, 5H), 7.78 (d, J=8.6 Hz, 1H), 7.61 (d, J=8.6 Hz, 1H), 7.59 (d, J=7.5 Hz, 1H), 7.46 (t, J=7.5 Hz, 1H), 7.32 (d, J=7.5 Hz, 1H), 6.93 (br s, 1H), 6.44 (br s, 2H), 6.29 (br s, 1H), 6.00 (br s, 1H).

### 7-Nitrobenzo[h]quinolinyl (tetrapyrazolylborate) palladium(IV) pyridine trifluorometahnesulfonate (3a)

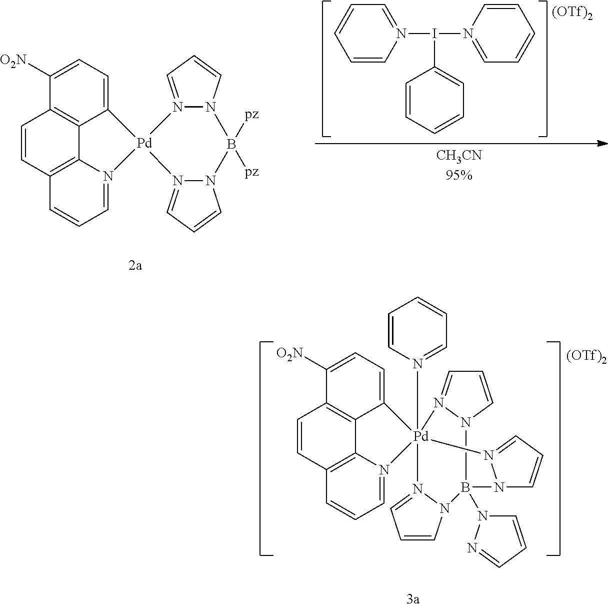

To 7-nitrobenzo[h]quinolinyl (tetrapyrazolylborate)palladium(II) (2a) (50.0 mg, 82.1 μmol, 1.00 equiv) in THF (3 mL) at 23° C. was added bis(pyridinio-1)iodobenzene bis(trifluoroacetate) (54.2 g, 82.1 μmol, 1.00 equiv). After stirring for 2 hrs the reaction mixture was concentrated in vacuo. The resulting residue was triturated with Et₂O (3×30 mL) and collected as a yellow solid to afford 78.0 mg of the title compound (96%). NMR. Spectroscopy: ¹H NMR (500 MHz, CD₃CN, 23° C., δ): 9.16 (d, J=7.5 Hz, 1H), 9.04 (d, J=6.4 Hz, 1H), 9.00 (d, J=10.7 Hz, 1H), 8.99 (s, 1H), 8.63 (d, J=9.6 Hz, 1H), 8.54 (d, J=8.6 Hz, 1H), 8.24-8.23 (m, 2H), 8.12-8.03 (m, 4H), 7.95 (d, J=3.2 Hz, 1H), 7.91 (d, J=8.6 Hz, 1H), 7.47 (d, J=5.3 Hz, 2H), 7.41 (d, J=3.2 Hz, 1H), 7.38 (t, J=7.5 Hz, 2H), 6.86 (t, J=2.1 Hz, 1H), 6.82 (t, J=2.1 Hz, 1H), 6.81 (t, J=2.1 Hz, 1H), 6.24 (s, J=2.1 Hz, 1H), 6.11 (t, J=3.1 Hz, 1H).

### Example 2

### Synthesis of Palladium(IV) Fluoride Complexes

### Benzo[h]quinolinyl (tetrapyrazolylborate) palladium(IV) fluoride trifluorometahnesulfonate (4)

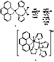

To benzo[h]quinolinyl (tetrapyrazolylborate)palladium(II) (2) (400 mg, 0.710 mmol, 1.00 equiv) dissolved in 15.0 mL CH₂Cl₂ was added XeF₂ (120.1 mg, 0.710 mmol, 1.00 equiv) in one portion at −30° C. After the solution was stirred for 30 min at −30° C., silver triflate (182.3 mg, 0.710 mmol, 1.00 equiv) was added to the solution at −30° C. After being stirred for 10 min at −30° C., the orange solution was stirred further at room temperature for 30 min. The solution was filtered through Celite and the filtrate was concentrated in vacuo. The residual was triturated with Et₂O (3×5 mL) to afford 460 mg of the title compound as an orange solid (89%). NMR Spectroscopy: ¹H NMR (500 MHz, CD₃CN, 23° C., δ): 9.01 (d, J=5.3 Hz, 1H), 8.96 (d, J=7.5 Hz, 1H), 8.78 (d, J=2.1 Hz, 1H), 8.432 (s, 2H), 8.28 (d, J=11.7 Hz, 1H), 8.27 (s, 1H), 8.23-8.19 (m, 2H), 8.16 (s, 1H), 8.06 (s, 1H), 7.96 (t, J=7.1 Hz, 1H), 7.82 (t, J=8.0 Hz, 1H), 7.62 (d, J=7.4 Hz, 1H), 6.78 (s, 2H), 6.74 (d, J=11.7 Hz, 2H), 6.54 (s, 1H), 6.11 (s, 1H). ¹⁹F NMR (375 MHz, CD₃CN, 23° C., δ): −79.7 (s), −319.5 (s).

### 7-Nitrobenzo[h]quinolinyl (tetrapyrazolylborate) palladium(IV) fluoride trifluorometahnesulfonate (4a)

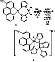

To 7-nitrobenzo[h]quinolinyl (tetrapyrazolylborate)palladium(II) (2a) (248 mg, 0.407 mmol, 1.00 equiv) dissolved in 40.0 mL CH₂Cl₂ was added XeF₂ (69.0 mg, 0.407 mmol, 1.00 equiv) in one portion at −30° C. After the solution was stirred for 2 hrs at −30° C., silver triflate (104.7 mg, 0.710 mmol, 1.00 equiv) was added to the solution at −30° C. After being stirred for 10 min at −30° C., the light yellow solution was stirred further at room temperature for 2 hrs. The solution was filtered through Celite and the filtrate was concentrated in vacuo. The residual was triturated with Et₂O (3×5 mL) to afford 262 mg of the title compound as a yellow solid (89%). NMR Spectroscopy: ¹H NMR (500 MHz, CD₃CN, 23° C., δ): 9.10 (d, J=5.3 Hz, 1H), 9.06 (d, J=7.5 Hz, 1H), 8.91 (d, J=9.6 Hz, 1H), 8.81 (d, J=2.1 Hz, 1H), 8.59 (d, J=8.5 Hz, 1H), 8.46 (d, J=9.6 Hz, 1H), 8.35 (d, J=2.1 Hz, 1H), 8.31 (s, 1H), 8.27 (d, J=2.1 Hz, 1H), 8.15 (s, 1H), 8.11-8.08 (m, 1H), 8.07 (s, 1H), 7.82 (d, J=8.6 Hz, 1H), 6.81-6.74 (m, 4H), 6.62 (d, J=2.1 Hz, 1H), 6.15 (s, 1H). ¹⁹F NMR (375 MHz, CD₃CN, 23° C., δ): −79.7 (s), −317.9 (s).

### Aryl Palladium Complex 6

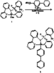

To the acetato palladium complex 5 (875.0 mg, 1.46 mmol, 1.00 equiv)⁴ in MeOH (20.0 mL) and benzene (20.0 mL) at 23° C. was added 4-biphenylboronic acid (318.2 mg, 1.61 mmol, 1.10 equiv) and K₂CO₃ (403.8 mg, 2.92 mmol, 2.00 equiv). The reaction mixture was stirred at 23° C. for 3.0 h, and the solvent was removed in vacuo. To the solid residue was added CHCl₃ (50 mL) and water (50 mL). The phases were separated and the aqueous phase was extracted with CHCl₃ (3×30 mL). The combined organic phases were washed with brine (5 mL) and dried (Na₂SO₄). The filtrate was concentrated in vacuo and the residue was purified by chromatography on silica gel eluting with CH₂Cl₂/MeOH 99:1 (v/v) to afford 250 mg of the title compound as a light yellow solid (25% yield). NMR Spectroscopy: ¹H NMR (500 MHz, CDCl₃, 23° C., δ): 8.91 (d, J=5.4 Hz, 2H), 8.23 (d, J=5.5 Hz, 1H), 7.75-7.65 (m, 1H), 7.56-7.47 (m, 5H), 7.43-7.24 (m, 10H), 7.18-7.05 (m, 6H), 6.83 (t, J=6.9 Hz, 1H).

### Example 3

### Fluoroination of Aryl Palladium Complex 6

Fluorination of 6 with 3 (or 3a) and KF

To KF (1.0 mg, 17.21 μmol, 1 equiv) and 18-crown-6 (4.6 mg, 17.21 gmol, 1 equiv) in CH₃CN (0.5 mL) at 23° C. was added 3 (or 3a, 1.5 equiv). After stirring for 30 min at 23° C., the volatiles were removed under vacuo. To the residue was added CH₂Cl₂ (1.0 mL) and 6 (37.2 mg, 0.105 mmol, 1.05 equiv) and the reaction mixture was stirred for 2 hrs at 60° C. The yields were determined by comparing the integration of the ¹⁹F NMR (375 MHz, CH₂Cl₂, 23° C.) resonance of the product 4-fluoro-1,1′-biphenyl and that of 4-nitrofluorobenzene (−103.9 ppm) based on KF as a limiting reagent. The average yields of two runs are reported in Table 1.

TABLE 1
fluorination of 6 with 3 (or 3a) and KF

| | Pd(IV) | | |
|---|---|---|---|
| Entry | complex | additives | Yield (%) |
| 1 | 3 | — | 50 |

Fluorination of 6 with 4 and 4a

To 4 (or 4a, 13.7 μmmol, 1 equiv) in CH₂Cl₂ (1.0 mL) at 23° C. was added 6 (1.5 equiv). The reaction mixture was stirred for 2.0 hr at 60° C. The yields were determined by comparing the integration of the ¹⁹F NMR (375 MHz, CH₂Cl₂, 23° C.) resonance of the product 4-fluoro-1,1′-biphenyl and that of 4-nitrofluorobenzene (−103.9 ppm). The average yields of two runs are reported in Table 2.

TABLE 2
fluorination of 6 with 4 (or 4a) and KF

| | Pd(IV) | | |
|---|---|---|---|
| Entry | complex | additives | Yield (%) |
| 1 | 4 | — | 60 |
| 2 | 4a | — | 70 |

### X-Ray Crystal Structure of 3.

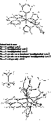

### Example 4

### Benzo[h]quinolinyl (tetrapyrazolylborate) palladium(IV) pyridine trifluoromethanesulfonate and benzo[h]quinolinyl (tetrapyrazolylborate) palladium(IV) fluoride trifluoromethanesulfonate

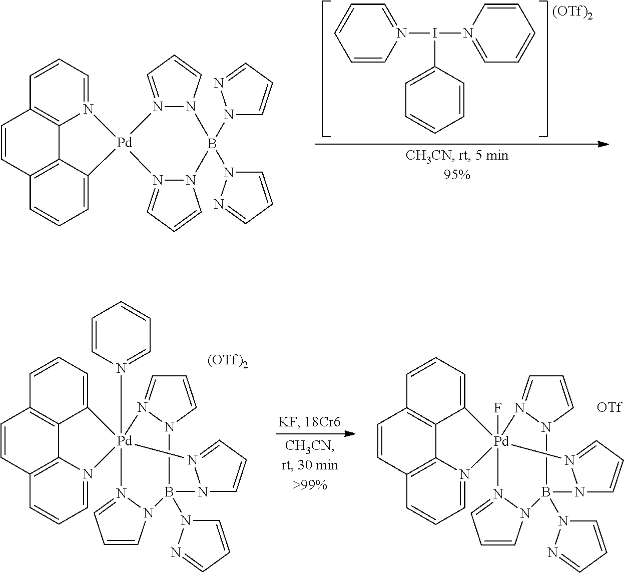

To benzo[h]quinolinyl (tetrapyrazolylborate)palladium(II) (1.00 g, 1.774 mmol, 1.00 equiv) in CH₃CN (13 mL) at 23° C. was added bis(pyridinio-1)iodobenzene bis(trifluoroacetate)⁵ (1.195 g, 1.81 mmol, 1.02 equiv). After stirring for 20 min the reaction mixture was concentrated in vacuo. The resulting residue was triturated with THF (3×30 mL) and collected as a light brown solid. The solid was triturated with pentane (3×30 mL) and collected to afford 1.580 g of the title compound as a light brown solid (95%). NMR Spectroscopy: ¹H NMR (500 MHz, CD₃CN, 23° C., δ): 9.07 (d, J=7.5 Hz, 1H), 8.98 (d, J=2.1 Hz, 1H), 8.96 (d, J=6.4 Hz, 1H), 8.43 (dd, J=39.5 Hz, J=9.6 Hz, 2H), 8.37 (d, J=7.5 Hz, 1H), 8.24 (d, J=2.1 Hz, 2H), 8.11-7.94 (m, 5H), 7.84 (t, J=8.0 Hz, 1H), 7.74 (d, J=8.6 Hz, 1H), 7.51 (s, 1H), 7.50 (s, 1H), 7.39-7.36 (m, 3H), 6.85 (t, J=2.1 Hz, 1H), 6.80 (s, 2H), 6.20 (s, J=2.1 Hz, 1H), 6.09 (t, J=2.1 Hz, 1H). To benzo[h]quinolinyl (tetrapyrazolylborate) palladium(IV) pyridine trifluorometahnesulfonate (2) (10.0 mg, 11.6 μmol, 1.00 equiv) in CD₃CN (0.6 mL) at 23° C. were added KF (0.7 mg, 12.1 μmol, 1.04 equiv) and 18-crown-6 (3.2 mg, 12.1 μmol, 1.04 equiv). Benzo[h]quinolinyl (tetrapyrazolylborate) palladium(IV) fluoride trifluorometahnesulfonate (3) was obtained quantitatively in 30 min, which was monitored by ¹H and ¹⁹F NMR spectroscopy using 4-nitrofluorobenzene as an internal standard. ⁵ Weiss, R.; Seubert, J. Angew. Chem., Int. Ed. 1994, 33, 891-893.

### Example 5

### Benzo[h]quinolinyl (tetrapyrazolylborate) palladium(IV) nitrate trifluoromethanesulfonate and benzo[h]quinolinyl (tetrapyrazolylborate) palladium(IV) fluoride trifluoromethanesulfonate

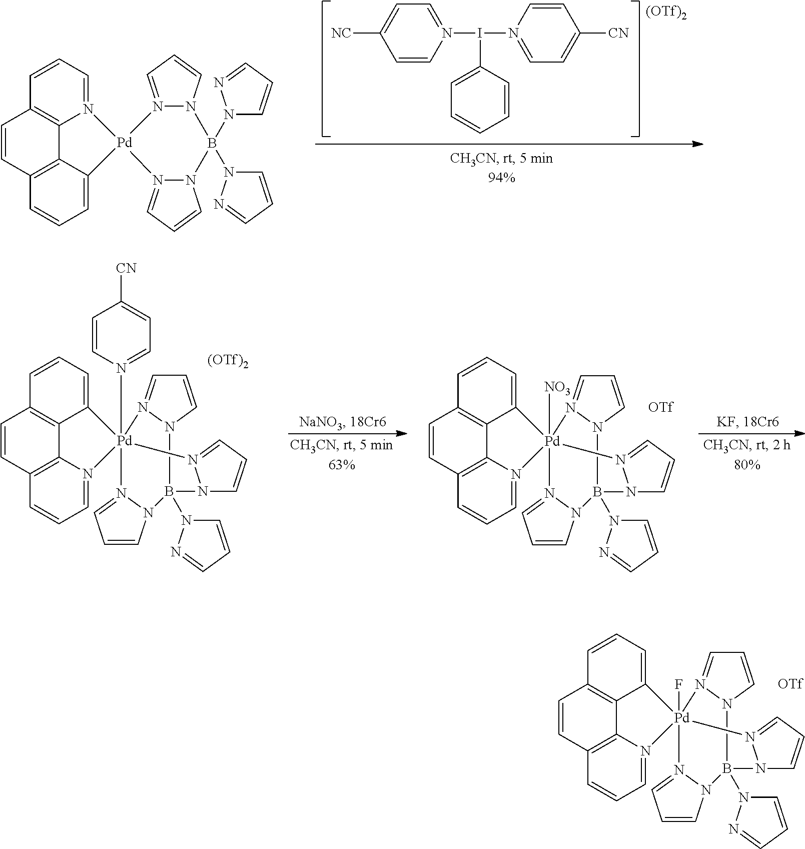

To benzo[h]quinolinyl (tetrapyrazolylborate)palladium(II) (300 mg, 0.55 mmol, 1.00 equiv) in CH₃CN (10 mL) at 23° C. was added bis(4-cyanopyridinio-1)iodobenzene bis(trifluoroacetate) (404.7 mg, 0.57 mmol, 1.03 equiv). After stirring for 5 min the reaction mixture was concentrated in vacuo. The resulting residue was triturated with THF (3×30 mL) and collected to afford 500 mg of benzo[h]quinolinyl (tetrapyrazolylborate) palladium(IV) 4-cyanopyridine trifluorometahnesulfonate as a light brown solid (94%). NMR Spectroscopy: ¹H NMR (400 MHz, CD₃CN, 23° C., δ): 9.07 (d, J=7.8 Hz, 1H), 8.99 (d, J=2.6 Hz, 1H), 8.94 (d, J=5.7 Hz, 1H), 8.44 (dd, J=31.1 Hz, J=9.0 Hz, 2H), 8.38 (d, J=7.9 Hz, 1H), 8.25 (d, J=11.0 Hz, 1H), 8.24 (d, J=11.2 Hz, 1H), 8.08 (d, J=1.3 Hz, 1H), 8.05 (d, J=3.0 Hz, 1H), 7.98-7.93 (m, 2H), 7.84 (t, J=8.0 Hz, 1H), 7.76-7.69 (m, 5H), 7.41 (d, J=3.2 Hz, 1H), 6.85 (t, J=2.5 Hz, 1H), 6.81-6.79 (m, 2H), 6.19 (d, J=2.6 Hz, 1H), 6.09 (t, J=2.6 Hz, 1H). To benzo[h]quinolinyl (tetrapyrazolylborate) palladium(IV) 4-cyanopyridine trifluorometahnesulfonate (131.8 mg, 0.14 mmol, 1.00 equiv) in CH₃CN (5 mL) at 23° C. were added sodium nitrate (11.8 mg, 0.14 mmol, 1.03 equiv) and 18-crown-6 (36.0 mg, 0.14 mmol, 1.00 equiv). After stirring for 5 min the reaction mixture was filtered through Celite and the filtrate was concentrated in vacuo. The resulting residue was triturated with ether (3×30 mL) and collected to afford 75.4 mg of benzo[h]quinolinyl (tetrapyrazolylborate) palladium(IV) nitrate trifluorometahnesulfonate as a brown solid (63%). NMR Spectroscopy: ¹H NMR (500 MHz, CD₃CN, 23° C., δ): 9.10 (d, J=5.8 Hz, 1H), 8.97 (d, J=8.6 Hz, 1H), 8.81 (d, J=2.4 Hz, 1H), 8.30-8.21 (m, 6H), 8.06 (s, 1H), 7.98 (dd, J=7.4 Hz, J=2.0 Hz, 1H), 7.82 (t, J=8.2 Hz, 1H), 7.60 (d, J=8.2 Hz, 1H), 6.98 (d, J=2.3 Hz, 1H), 6.80 (t, J=3.0 Hz, 1H), 6.77 (s, 1H), 6.74 (t, J=2.1 Hz, 1H), 6.37 (d, J=2.5 Hz, 1H), 6.10 (t, J=3.0 Hz, 1H). To benzo[h]quinolinyl (tetrapyrazolylborate) palladium(IV) nitrate trifluorometahnesulfonate (13.0 mg, 15.5 μmol, 1.00 equiv) in CD₃CN (0.6 mL) at 23° C. were added KF (0.9 mg, 12.1 μmol, 1.05 equiv) and 18-crwon-6 (4.1 mg, 12.1 μmol, 1.05 equiv). Benzo[h]quinolinyl (tetrapyrazolylborate) palladium(IV) fluoride trifluorometahnesulfonate was obtained in 2 hrs (80% yield), which was monitored by ¹H and ¹⁹F NMR spectroscopy using 4-nitrofluorobenzene as an internal standard.

### Example 6

### 1,1′-(phenyl-λ³-iodanediyl)bis(4-cyanopyridinium)bis(trifluoromethanesulfonate) (S3)

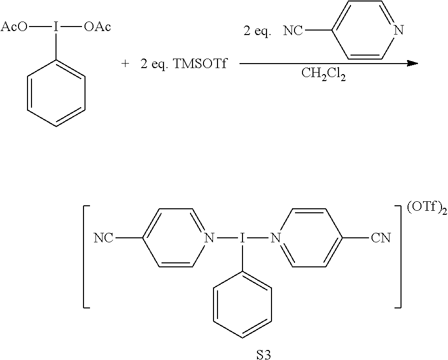

All manipulations were carried out in a dry box under a N₂ atmosphere. To (diacetoxyiodo)benzene (2.00 g, 6.21 mmol, 1.00 equiv) dissolved in 100 mL CH₂Cl₂ was added TMSOTf (2.83 g, 12.7 mmol, 2.00 equiv) slowly at 23° C. 4-Cyanopyridine (1.29 g, 12.7 mmol, 2.00 equiv) in 10 mL CH₂Cl₂ was added to the solution dropwise to give a colorless precipitate and the mixture was stirred for 30 min vigorously at 23° C. The solid was filtered off and washed with 10 mL CH₂Cl₂ three times and subsequently dried under vacuum to afford 3.80 g of the title compound as a colorless solid (86%). NMR Spectroscopy: ¹H NMR (500 MHz, CD₃CN, +23° C., δ): 9.21 (d, J=5.3 Hz, 4H), 8.74 (d, J=7.5 Hz, 2H), 8.11 (d, J=6.4 Hz, 4H), 7.87 (t, J=7.5 Hz, 1H), 7.71 (t, J=8.0 Hz, 2H). ¹³C NMR (125 MHz, CD₃CN, +23° C., δ): 150.1, 137.4, 136.8, 134.7, 132.4, 128.8, 124.0, 121.9 (q, J=319 Hz, triflate), 115.4. ¹⁹F NMR (375 MHz, CD₃CN, 23° C., δ): −77.5. Anal: calcd for C₂₀H₁₃F₆₁N₄O₆S₂: C, 33.82; H, 1.84; N, 7.89. found: C, 33.63; H, 1.67; N, 7.68.

### Benzo[h]quinolinyl (tetrapyrazolylborate) Pd(IV) pyridine trifluoromethanesulfonate (SB)

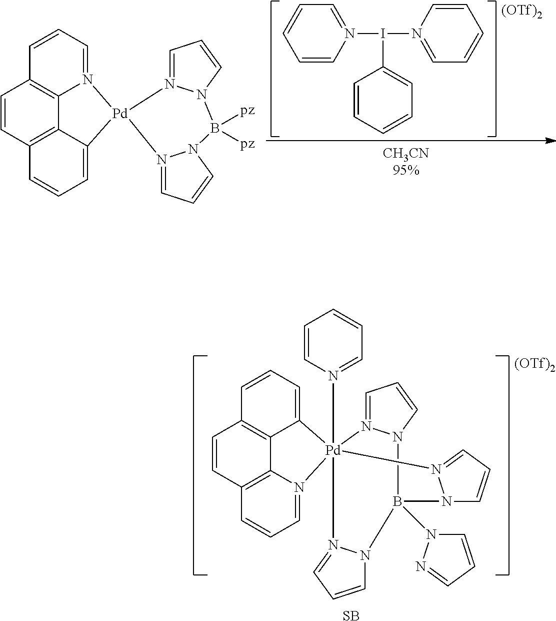

All manipulations were carried out in a dry box under a N₂ atmosphere. To benzo[h]quinolinyl (tetrapyrazolylborate)palladium (5) (1.00 g, 1.77 mmol, 1.00 equiv) in CH₃CN (13 mL) at 23° C. was added phenyl(dipyridinium)iodonium bis(trifluoromethanesulfonate) (SA) (1.20 g, 1.81 mmol, 1.02 equiv). After stirring for 20 min the reaction mixture was concentrated in vacuo. The resulting residue was triturated with THF (30 mL) and collected on a frit by filtration as a light brown solid. The solid was redissolved in 5 mL CH₃CN and volatiles including residual THF were removed in vacuo to afford 1.58 g of the title compound as a brown solid (95%). NMR Spectroscopy: ¹H NMR (500 MHz, CD₃CN, 23° C., δ): 9.07 (d, J=7.5 Hz, 1H), 8.98 (d, J=2.1 Hz, 1H), 8.96 (d, J=6.4 Hz, 1H), 8.49 (d, J=9.0 Hz, 1H), 8.43 (d, J=9.0 Hz, 1H), 8.37 (d, J=7.5 Hz, 1H), 8.24 (d, J. 2.1 Hz, 2H), 8.11-7.94 (m, 5H), 7.84 (t, J. 8.0 Hz, 1H), 7.74 (d, J=8.6 Hz, 1H), 7.51 (s, 1H), 7.50 (s, 1H), 7.39-7.36 (m, 3H), 6.85 (t, J=2.1 Hz, 1H), 6.80 (s, 2H), 6.20 (d, J. 2.1 Hz, 1H), 6.09 (t, J=2.1 Hz, 1H). ¹⁹F NMR (375 MHz, CD₃CN, 23° C., δ): −77.5. ¹³C NMR (125 MHz, CDCl₃, 23° C., δ): 169.4, 152.2, 152.1, 148.4, 145.0, 144.4, 144.3, 144.1, 144.0, 142.6, 140.6, 140.2, 139.9, 139.6, 137.7, 134.2, 133.6, 133.4, 131.7, 130.2, 130.1, 130.0, 128.6, 126.9, 121.9 (q, J=319 Hz, triflate), 112.1, 110.3, 109.6. Anal: calcd for C₃₂H₂₅BF₆N₁₀O₆PdS₂: C, 40.85; H, 2.68; N, 14.89. found: C, 40.84; H, 2.81; N, 14.89. X-ray quality crystals were obtained from 1.0 mL CH₃CN solution that contained 50 mg of the title compound layered slowly with 0.5 mL Et₂O at 23° C. For crystallography data, see X-ray section.

### Benzo[h]quinolinyl (tetrapyrazolylborate) Pd(IV) 4-cyanopyridine trifluoromethanesulfonate (S4)

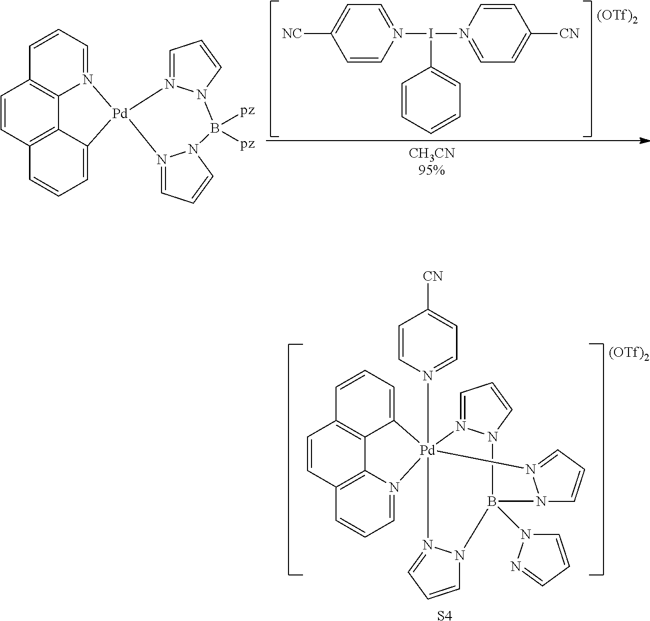

All manipulations were carried out in a dry box under a N₂ atmosphere. To benzo[h]quinolinyl (tetrapyrazolylborate)palladium (3.00 g, 5.32 mmol, 1.00 equiv) in CH₃CN (50 mL) at 23° C. was added 1,1′-(phenyl-λ³-iodanediyl)bis(4-cyanopyridinium)bis(trifluoromethanesulfonate) (S3) (3.98 g, 5.48 mmol, 1.03 equiv). After stirring for 30 min the reaction mixture was concentrated in vacuo. The resulting residue was triturated with THF (3×30 mL) and collected on a frit by filtration as a light brown solid. The solid was re-dissolved in 10 mL CH₃CN and volatiles including residual THF were removed in vacuo to afford 4.80 g of the title compound as a brown solid (93%). NMR Spectroscopy: ¹H NMR (500 MHz, CD₃CN, +23° C., δ): 9.07 (d, J=7.5 Hz, 1H), 8.98 (d, J=2.1 Hz, 1H), 8.96 (d, J=6.4 Hz, 1H), 8.47 (d, J=9.6 Hz, 1H), 8.40 (d, J=9.6 Hz, 1H), 8.37 (d, J=7.5 Hz, 1H), 8.24 (d, J=2.1 Hz, 2H), 8.11-7.94 (m, 5H), 7.84 (t, J=8.0 Hz, 1H), 7.74 (d, J=8.6 Hz, 1H), 7.51 (s, 1H), 7.50 (s, 1H), 7.39-7.36 (m, 3H), 6.85 (t, J=2.1 Hz, 1H), 6.80 (s, 2H), 6.20 (d, J=2.1 Hz, 1H), 6.09 (t, J=2.1 Hz, 1H). ¹³C NMR (125 MHz, CD₃CN, +23° C.): 169.5, 153.5, 152.3, 148.2, 144.5, 144.4, 144.1, 144.0, 142.7, 140.8, 140.4, 140.0, 139.8, 137.7, 134.0, 133.7, 133.5, 132.0, 131.7, 130.4, 130.3, 128.7, 127.7, 127.0, 121.9 (q, J=319 Hz, triflate), 115.1, 112.2, 110.5, 110.5, 110.4, 109.6. ¹⁹F NMR (375 MHz, CD₃CN, +23° C., δ): −77.5. Anal: calcd for C₃₃H₂₄BF₆N₁₁O₆PdS₂: C, 41.03; H, 2.50; N, 15.95. found: C, 40.78; H, 2.47; N, 15.67.

### Benzo[h]quinolinyl (tetrapyrazolylborate) Pd(IV) 4-picoline trifluoromethanesulfonate (10)

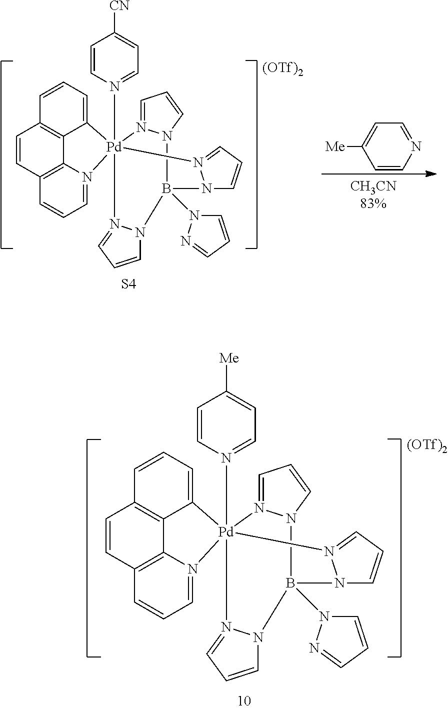

All manipulations were carried out in a dry box under a N₂ atmosphere. To benzo[h]quinolinyl (tetrapyrazolylborate) Pd(IV) 4-cyanopyridine trifluoromethanesulfonate (S4) (5.00 g, 5.16 mmol, 1.00 equiv) in CH₃CN (15 mL) at 23° C. was added 4-picoline (769 mg, 8.26 mmol, 1.60 equiv). After stirring for 2 min the reaction mixture was added dropwise to 200 mL of Et₂O while stirring vigorously at 23° C. The resulting precipitate was collected on a frit by filtration as a light brown solid. The solid was washed with Et₂O (3×30 mL) and subsequently dried to afford 4.40 g of the title compound as a brown solid (89%). NMR Spectroscopy: ¹H NMR (500 MHz, CD₃CN, +23° C., δ): 9.05 (d, J=7.9 Hz, 1H), 8.98 (d, J=2.4 Hz, 1H), 8.94 (d, J=5.5 Hz, 1H), 8.46 (d, J=8.5 Hz, 1H), 8.38 (d, J=9.2 Hz, 1H), 8.36 (d, J=7.9 Hz, 1H), 8.23 (d, J=2.5 Hz, 1H), 8.21 (d, J=1.8 Hz, 1H), 8.08 (d, J=1.2 Hz, 1H), 8.03 (d, J=2.4 Hz, 1H), 7.95-7.91 (m, 2H), 7.83 (t, J=7.9 Hz, 1H), 7.72 (d, J=8.5 Hz, 1H), 7.38 (d, J=2.3 Hz, 1H), 7.30 (d, J=7.3 Hz, 1H), 7.17 (d, J=6.7 Hz, 1H), 6.85 (t, J=2.1 Hz, 1H), 6.80-6.78 (m, 2H), 6.18 (d, J=2.4 Hz, 1H), 6.08 (t, J=2.4 Hz, 1H), 2.38 (s, 3H). ¹³C NMR (125 MHz, CD₃CN, +23° C., δ): 169.2, 158.7, 152.0, 151.1, 148.5, 144.4, 144.3, 144.1, 143.9, 142.6, 140.6, 140.2, 139.9, 139.6, 137.7, 134.3, 133.5, 133.4, 131.7, 130.4, 130.2, 130.0, 128.6, 126.9, 121.9 (q, J=319 Hz, triflate), 112.0, 110.3, 110.3, 109.6, 21.2. ¹⁹F NMR (375 MHz, CD₃CN, +23° C., δ): −77.5. Anal: calcd for C₃₃H₂₇BF₆N₁₀O₆PdS₂: C, 41.50; H, 2.85; N, 14.67. found: C, 41.45; H, 2.72; N, 14.41. X-ray quality crystals were obtained from 1.0 mL CH₃CN solution that contained 20 mg of the title compound layered slowly with 0.5 mL Et₂O at 23° C. For crystallography data, see X-ray section.

### Example 7

### Benzo[h]quinolinyl (tetrapyrazolylborate) Pd(IV) fluoride trifluoromethanesulfonate (4)

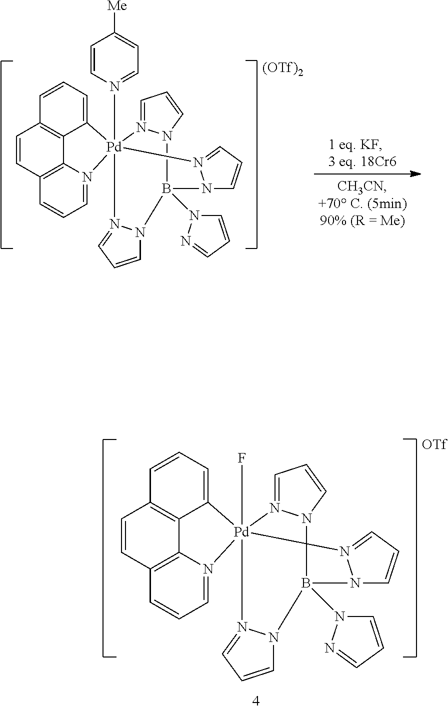

In a glove box, to benzo[h]quinolinyl (tetrapyrazolylborate) Pd(IV) 4-picoline trifluoromethanesulfonate (10) (284 mg, 0.297 mmol, 1.00 equiv) dissolved in 15 mL CH₃CN in a soda lime glass bottle was added KF (17.3 mg, 0.297 mmol, 1.00 equiv) and 18-crown-6 (235 mg, 0.891 mmol, 3.00 equiv) in one portion at 23° C. The bottle was sealed, taken out of the glove box, sonicated at 23° C. for 5 minutes, immersed in a oil bath heated at 50° C., and the solution was vigorously stirred for 5 minutes. CH₃CN (10 mL) was added to the solution, and the solution was filtered through Celite, eluting with an additional 10 mL of CH₃CN. The filtrate was concentrated in vacuo. The residue was triturated with THF (3×15 mL) and subsequently dried in vacuo to afford 195 mg of the title compound as an orange solid (90%). A large-scale reaction: To benzo[h]quinolinyl (tetrapyrazolylborate) Pd(IV) pyridine trifluoromethanesulfonate (SB) (7.80 g, 8.29 mmol, 1.00 equiv) dissolved in 150 mL CH₃CN was added KF (0.540 g, 9.26 mmol, 1.12 equiv) and 18-crwon-6 (0.160 g, 0.62 mmol, 0.0700 equiv) in one portion at 23° C. After the solution was vigorously stirred for 3 days at 23° C. and another 350 mL of CH₃CN was added to the reaction solution. The solution was warmed to +50° C. until the turbid solution became clear and the solution was filtered through Celite eluting with 100 mL CH₃CN. The filtrate was concentrated in vacuo. The residual solid was triturated with THF (3×50 mL), filtered off, and subsequently dried in vacuo to afford 5.80 g of the title compound as an orange solid (94%). NMR Spectroscopy: ¹H NMR (500 MHz, CD₃CN, +23° C., δ): 9.01 (d, J=5.3 Hz, 1H), 8.96 (d, J=7.5 Hz, 1H), 8.78 (d, J=2.1 Hz, 1H), 8.43 (s, 2H), 8.28 (d, J=11.7 Hz, 1H), 8.27 (s, 1H), 8.23-8.19 (m, 2H), 8.16 (s, 1H), 8.06 (s, 1H), 7.96 (t, J=7.1 Hz, 1H), 7.82 (t, J=8.0 Hz, 1H), 7.62 (d, J=7.4 Hz, 1H), 6.78 (s, 2H), 6.74 (d, J=11.7 Hz, 2H), 6.54 (s, 1H), 6.11 (s, 1H). ¹³C NMR (125 MHz, DMSO-d₆, +23° C., δ): 165.0, 149.4, 149.2, 149.4, 149.2, 143.4, 143.0, 142.7, 142.7, 142.2, 138.5, 137.6, 137.6, 137.0, 136.7, 134.8, 132.1, 130.3, 129.6, 127.6, 127.6, 126.4, 120.7 (q, J=323 Hz, triflate), 109.9, 109.6, 108.5, 108.4. ¹⁹F NMR (375 MHz, CD₃CN, +23° C., δ): −77.5 (s), −317.3 (s). Anal: calcd for C₂₆H₂₀BF₄N₉O₃PdS: C, 42.67; H, 2.75; N, 17.23. found: C, 42.95; H, 2.95; N, 17.04. X-ray quality crystals were obtained from 4 mL CH₃CN solution that contained 20.0 mg of the title compound slowly layered with 3.0 mL Et₂O at 23° C. For crystallography data, see X-ray section.

Thermal stability of 4: 4 was placed in a vial and heated for 24 h at 100° C. under dynamic vacuum (10⁻⁴ Torr). The solid was analyzed by ¹H and ¹⁹F NMR spectroscopy, and showed no decomposition.

Tolerance of 4 toward water: 2.4 mg of 4 (3.3 μmol) and 2.0 μL of THF (internal standard) were dissolved in 0.55 mL of CD₃CN in a NMR tube. D₂O (63 μL) was added to the solution. The solution was kept at +23° C. for 3 hours and monitored by ¹H and ¹⁹F NMR spectroscopy, which showed no decomposition (Figure S1).

### Benzo[h]quinolinyl (tetrapyrazolylborate) Pd(IV) fluoride trifluoromethanesulfonate (4)

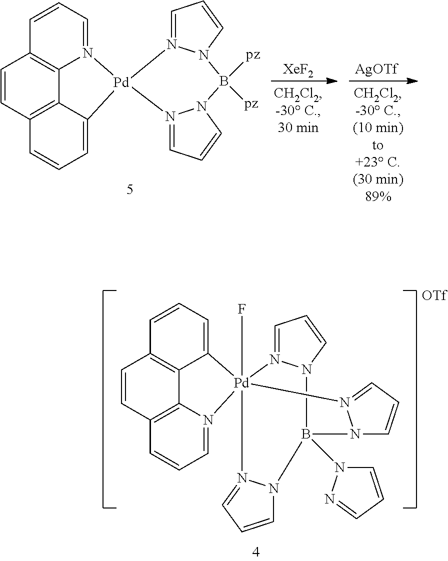

In a glove box, to benzo[h]quinolinyl (tetrapyrazolylborate)palladium (5) (400 mg, 0.710 mmol, 1.00 equiv) dissolved in 15 mL CH₂Cl₂ was added XeF₂ (120 mg, 0.710 mmol, 1.00 equiv) in one portion at −30° C. After the solution was stirred for 30 min at −30° C., silver triflate (182 mg, 0.710 mmol, 1.00 equiv) was added to the solution at −30° C. After being stirred for 10 min at −30° C., the orange solution was stirred further at 23° C. for 30 min. The solution was filtered through Celite eluting with 10 mL CH₂Cl₂ and the filtrate was concentrated in vacuo. The residual solid was triturated with Et₂O (3×5 mL) to afford 460 mg of the title compound as an orange solid (89%). The NMR spectroscopic data corresponds to that reported above for compound 4.

### Example 8

### 1-(1-Pyrrolidino)-3,4-dihydronaphthalene (16)

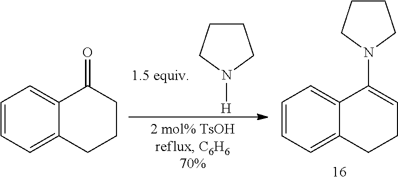 [SMILES: O=C1CCCc2ccccc21 ; C1CCNC1 ; C1=C(N2CCCC2)c2ccccc2CC1]

⁶ R. G. Harvey, of al., J. Org. Chem. 56, 1210 (1991).

To a solution of a-tetralone (3.90 g, 171 mmol, 1.00 equiv) in 50 mL of benzene were added pyrrolidine (2.85 g, 256 mmol, 1.50 equiv) and p-toluenesulfonic acid (100 mg, 0.526 mmol, 0.0184 equiv). The solution was heated at reflux for 2 days with azeoptropic removal of water using a Dean Stark trap. After cooling, the solvent was removed in vacuo and the residue was fractionally distilled (bp. 100° C./0.01 Torr) under reduced pressure gave the title compound as colorless liquid (3.70 g, 70%). NMR Spectroscopy: ¹H NMR (500 MHz, C₆D₆, +23° C., δ): 7.62 (d, J=7.7 Hz, 1H), 7.26-7.22 (m, 1H), 7.10-7.06 (m, 2H), 5.11 (t, J=4.7 Hz, 1H), 2.85 (t, J=5.7 Hz, 2H), 2.59 (t, J=7.1 Hz, 1H), 2.12 (td, J=7.1 Hz, J=4.8 Hz, 1H) 1.62 (quin, J=2.9 Hz, 2H). ¹³C NMR (125 MHz, C₆D₆, +23° C., δ): 146.1, 138.3, 132.8, 127.7, 126.9, 126.4, 124.7, 104.5, 50.9, 50.7, 50.5, 29.6, 24.2, 23.3. HRMS-FIA (m/z): calcd for C₁₄H₁₈N [M+H]⁺, 200.14338; found, 200.14340.

### 2-Fluoro-1-tetralone (rac-4)

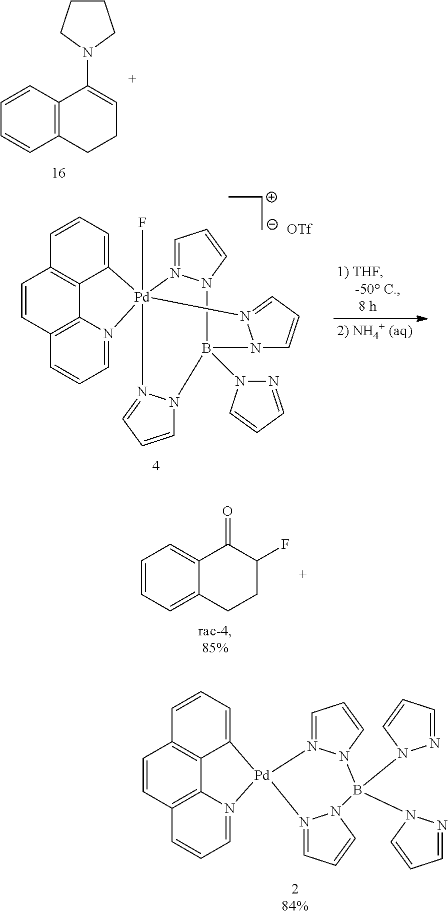

In a glove box, solution of 1-(1-pyrrolidino)-3,4-dihydronaphthalene (16) (20.0 mg, 0.100 mmol, 1.00 equiv) in 3 mL of THF was stirred at −50° C. for 30 min. To the solution was added benzo[h]quinolinyl (tetrapyrazolylborate) Pd(IV) fluoride trifluoromethanesulfonate (4) (88.1 mg, 0.120 mmol, 1.20 equiv). The reaction mixture was stirred at −50° C. for 8 hours, slowly warmed to 23° C., and taken out of the glove box. After 0.5 mL of aqueous NH₄Cl solution (1 M) was added, the organic solvent was removed in vacuo. The residue was extracted from with Et₂O (3×3 mL). rac-4: The combined organic phases were filtered through Celite eluting with 5 mL of Et₂O. The filtrate was concentrated in vacuo and the residue was purified by chromatography on silica gel eluting with EtOAc/hexane 1:10 (v/v) to afford 14.0 mg of 2-fluoro-1-tetralone as a colorless oil (85% yield). 2: The residue was washed with H₂O (3×0.5 mL) and MeCN (0.5 mL) and triturated with Et₂O (3×2 mL) to afford 57.0 mg of benzo[h]quinolinyl (tetrapyrazolylborate)palladium (2) as an light yellow solid (84%).

TLC (hexanes/EtOAc 10:1, v/v): R_{F}=0.29; NMR Spectroscopy: ¹H NMR (500 MHz, CDCl₃, 23° C., δ): 8.07 (d, J=8.6 Hz, 1H), 7.53 (t, J=7.6 Hz, 1H), 7.36 (t, J=7.6 Hz, 1H), 7.27 (d, J=8.6 Hz, 1H), 5.15 (ddd, J=48.0 Hz, J=12.8 Hz, J=5.2 Hz, 1H), 3.13 (dd, J=5.1 Hz, J=4.1 Hz, 2H), 2.61-2.54 (m, 1H), 2.41-2.31 (m, 1H). ¹³C NMR (125 MHz, CDCl₃, 23° C., δ): 193.4 (d, J=14.5 Hz), 143.1, 134.3, 131.4, 128.8, 128.0(d, J=2 Hz), 127.3, 91.3 (d, J=189 Hz), 30.2 (d, J=20 Hz), 27.1 (d, J=12 Hz). ¹⁹F NMR (375 MHz, CD₃CN, 23° C., δ): −189.4 (dt, J=48.3 Hz, J=7.6 Hz). HRMS-FIA (m/z): calcd for C₁₀H₁₀FO [M+H]⁺, 165.07102. found, 165.07104.

### Example 9

### [{(4-Methoxyphenyl)sulfonyl}imino]phenyliodinane (S5)

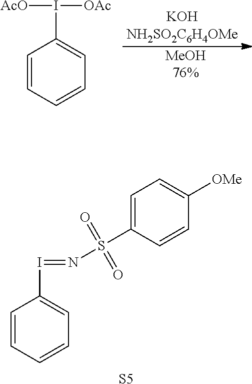

7 P. Muller, C. Baud, Y. Jacquier, Can. J. Chem. 76, 738 (1998).8 S. Taylor et al., J. Chem. Soc., Perkin Trans. 2 1714 (2001).

To p-methoxybenzenesulfonamide (5.00 g, 26.7 mmol, 1.00 equiv) in methanol (100 mL) at 23° C. was added potassium hydroxide (3.75 g, 66.8 mmol, 2.50 equiv). The reaction mixture was stirred at 23° C. for 10 min and subsequently cooled to 0° C. To the reaction mixture at 0° C. was added iodobenzene diacetate (8.60 g, 26.7 mmol, 1.00 equiv). The reaction mixture was stirred at 0° C. for 10 min and further stirred at 23° C. for 2.0 h. The reaction mixture was poured into cold water (700 mL) and kept at 0° C. for 4 h. The suspension was filtered off and the filter cake was washed with water (2×200 mL) and methanol (2×200 mL) to afford 7.90 g of the title compound as a colorless solid (76% yield).

NMR Spectroscopy: ¹H NMR (500 MHz, DMSO-d₆, 23° C., δ): 7.70 (d, J=7.5 Hz, 2H), 7.49-7.44 (m, 3H), 7.32-7.28 (m, 2H), 6.78 (d, J=8.5 Hz, 2H), 3.74 (s, 3H). ¹³C NMR (125 MHz, DMSO-d₆, 23° C., δ): 160.6, 136.9, 133.2, 130.5, 130.2, 128.0, 117.0, 113.4, 55.4. These spectroscopic data correspond to the reported data in reference⁸.

### [{(4-Nitrophenyl)sulfonyl}imino]phenyliodinane (SC)^{9,10}

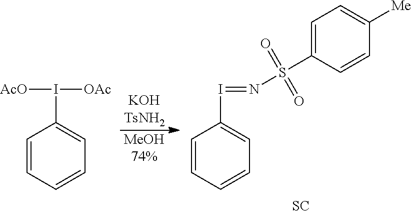

9Y. Yamada, T. Yamamoto, M. Okawara, Chem. Lett. 4, 361 (1975).10J. Gullick et al., New J. Chem. 28, 1470 (2004).

To p-toluenesulfonamide (5.00 g, 29.2 mmol, 1.00 equiv) in methanol (100 mL) at 23° C. was added potassium hydroxide (4.96 g, 73.0 mmol, 2.50 equiv). The reaction mixture was stirred at 23° C. for 10 min and cooled to 0° C. To the reaction mixture at 0° C. was added iodobenzene diacetate (9.41 g, 29.2 mmol, 1.00 equiv). The reaction mixture was stirred at 0° C. for 10 min and further stirred at 23° C. for 2.0 h. The reaction mixture was poured onto cold water (700 mL) and kept at 0° C. for 4 h. The suspension was filtered off and the filter cake was washed with water (2×200 mL) and methanol (2×200 mL) to afford 8.10 g of the title compound as a white solid (74% yield).

NMR Spectroscopy: ¹H NMR (500 MHz, DMSO-d₆, 23° C., δ): 7.68 (d, J=7.8 Hz, 2H), 7.47-7.43 (m, 3H), 7.31-7.28 (m, 2H), 7.06 (d, J=7.8 Hz, 2H), 2.26 (s, 3H). ¹³C NMR (125 MHz, DMSO-d₆, 23° C., δ): 142.1, 140.1, 133.2, 130.4, 130.2, 128.6, 126.1, 117.2, 20.8. These spectroscopic data correspond to the reported data in reference¹⁰.

### Benzo[h]quinolinyl palladium chloro dimer (S6)¹¹

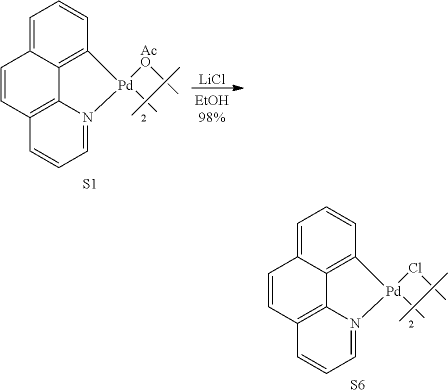

11 G. E. Hartwell, R. W. Lawrence, M. J. Smas, J. Chem. Soc. D.: Chem. Commun. 912 (1970).

To benzo[h]quinolinyl palladium acetate dimer (S1) (4.27 g, 12.4 mmol, 1.00 equiv) in EtOH (100 mL) at 0° C. was added lithium chloride (10.50 g, 24.8 mmol, 20.0 equiv). The reaction mixture was warmed to 23° C. and stirred for 1.0 h. The reaction mixture was filtered off and the filter cake was washed with water (3×100 mL), MeOH (2×100 mL), and Et₂O (100 mL) to afford 3.89 g of the title compound as a pale yellow solid (98% yield).

¹H NMR (500 MHz, DMSO-d₆, 23° C., δ): 9.44 (d, J=4.5 Hz, 1H), 8.72 (br), 8.67 (d, J=7.5 Hz, 1H), 8.61 (br), 8.22 (d, J=7.0 Hz, 1H), 7.91 (d, J=9.0 Hz, 1H), 7.86-7.74 (m, 3H), 7.73 (br), 7.60 (br), 7.53 (dd, J=7.5 Hz, J=7.0 Hz 1H), 7.38 (br); ¹³C NMR (125 MHz, DMSO-d₆, 23° C., δ): 153.9, 152.2, 150.7, 150.6, 148.0, 141.7, 139.9, 134.4, 130.8, 129.6, 129.4, 127.5, 125.1, 124.4, 123.0, 122.9. Note: The complicated ¹H and ¹³C NMR spectra were probably due to the mixture of the title compound and solvent adduct in DMSO-d₆. The title compound was not soluble in non-coordinating solvents.

### Chloro Palladium Complex (S7)¹²

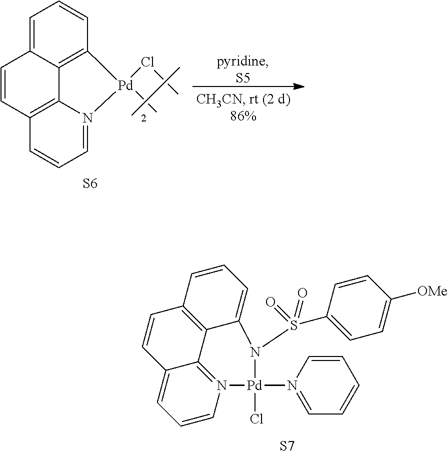

¹²A. R. Dick, M. S. Remy, J. W. Kampf, M. S. Sanford, Organometallics 26, 1365 (2007).

In a glove box, to chloropalladium dimer (S6) (6.00 g, 18.7 mmol, 1.00 equiv) in CH₃CN (100 mL) at 23° C. was added pyridine (6.06 mL, 75.0 mmol, 4.00 equiv) and [{(4-methoxyphenyl)sulfonyl}imino]phenyliodinane (S5) (10.9 g, 28.1 mmol, 1.50 equiv). The reaction mixture was stirred at 23° C. for 2 d and subsequently taken out of the glove box.

The reaction mixture was filtered off and the filter cake was washed with Et₂O (3×30 mL) to afford 9.70 g of the title compound as a yellow solid (86% yield).

¹H NMR (500 MHz, CDCl₃, 23° C., δ): 9.21 (dd, J=5.6 Hz, J=1.7 Hz, 1H), 9.00 (dd, J=6.8 Hz, J=1.5 Hz, 2H), 8.08 (dd, J=8.3 Hz, J=1.5 Hz, 1H), 7.87-7.73 (m, 5H), 7.47-7.44 (m, 3H), 7.35 (dd, J=7.8 Hz, J=5.4 Hz, 1H), 7.09 (dt, J=9.3 Hz, J=2.6 Hz, 2H), 6.17 (dt, J=8.4 Hz, J=2.4 Hz, 2H), 3.56 (s, 3H): ¹³C NMR (125 MHz, CDCl₃, 23° C., δ): 160.9, 154.2, 152.6, 141.9, 139.0, 138.6, 138.4, 136.0, 134.3, 130.4, 129.8, 128.4, 128.1, 127.7, 126.8, 125.6, 125.0, 124.2, 122.1, 112.4, 554. These spectroscopic data correspond to reported data¹².

### Chloro Palladium Complex (SD)¹²

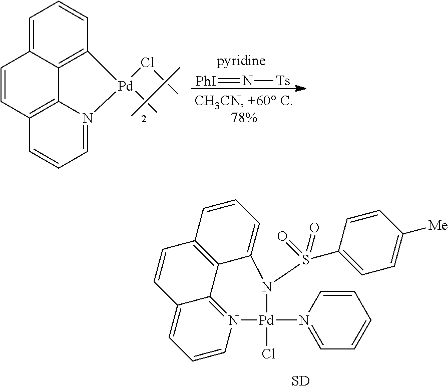

To chloropalladium dimer 3 (2.00 g, 6.25 mmol, 1.00 equiv) in CH₃CN (60.0 mL) at 23° C. was added pyridine (1.98 mL, 25.0 mmol, 4.00 equiv) and PhI═N-Ts (3.50 g, 9.37 mmol, 1.50 equiv). The reaction mixture was stirred at 60° C. for 3 h. The reaction mixture was filtered off and the filter cake was washed with Et₂O (2×10 mL) to afford 2.44 g of the title compound as a yellow solid (69% yield).

¹H NMR (500 MHz, CDCl₃, 23° C., δ): 9.17 (d, J=5.7 Hz, 1H), 9.01 (d, J=4.8 Hz, 2H), 8.07 (d, J=7.6 Hz, 1H), 7.88-7.75 (m, 5H), 7.48-7.44 (m, 3H), 7.34 (dd, J=7.6 Hz, J=5.7 Hz, 1H), 7.06 (d, J=7.6 Hz, 2H), 6.49 (d, J=7.6 Hz, 2H), 2.01 (s, 3H): ¹³C NMR (125 MHz, CDCl₃, 23° C., δ): 154.2, 152.6, 141.9, 140.7, 139.3, 138.9, 138.7, 138.2, 136.0, 130.4, 129.8, 128.4, 127.9, 127.6, 126.7, 126.2, 125.6, 125.1, 124.2, 122.0, 21.1. These spectroscopic data correspond to the reported data in reference¹².

### Acetato Palladium Complex (S8)

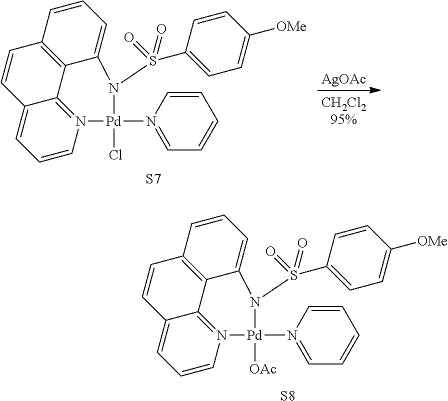

To chloro palladium complex (S7) (5.00 g, 8.34 mmol, 1.00 equiv) in CH₂Cl₂ (300 mL) at 23° C. was added AgOAc (4.87 g, 29.2 mmol, 3.50 equiv). The suspension was stirred at 40° C. for 3 h. After cooling to 23° C., the suspension was filtered through a plug of Celite. The filtrate was concentrated in vacuo and the residue was triturated with Et₂O (100 mL). The solid was filtered off and washed with Et₂O (2×50 mL) to afford 5.07 g of the title compound as a yellow solid (95% yield).

¹H NMR (500 MHz, CDCl₃, 23° C., δ): 8.93 (d, J=4.9 Hz, 2H), 8.70 (dd, J=5.2 Hz, J=1.5 Hz, 1H), 8.01 (dd, J=7.9 Hz, J=1.2 Hz, 1H), 7.83 (dd, J=7.3 Hz, J=4.8 Hz, 1H), 7.80 (t, J=7.6 Hz, 1H) 7.74-7.68 (m, 3H), 7.41-7.35 (m, 3H), 7.27 (dd, J=7.6 Hz, J=5.2 Hz, 1H), 7.15 (t, J=8.5 Hz, 2H), 6.13 (d, J=8.5 Hz, 2H), 3.48 (s, 3H), 1.78 (s, 3H); ¹³C NMR (125 MHz, CDCl₃, 23° C., δ): 177.4, 160.7, 151.6, 151.2, 141.7, 139.0, 138.4, 138.2, 135.8, 134.4, 130.1, 129.9, 128.9, 128.1, 127.3, 126.7, 125.5, 124.8, 124.0, 121.8, 112.3, 55.2, 23.8. Anal: calcd for C₂₇H₂₃N₃O₅PdS: C, 53.34; H, 3.81; N, 6.91. found: C, 53.31; H, 3.69; N, 6.89¹².

### Acetato Palladium Complex (SE)

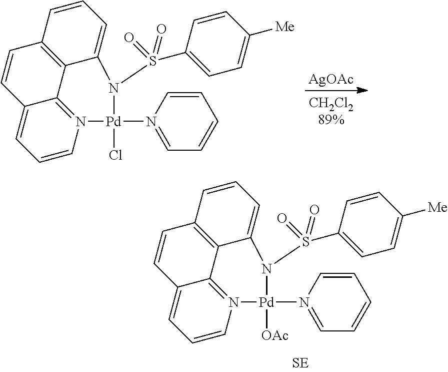

To chloro palladium complex (SD) (2.40 g, 4.22 mmol, 1.00 equiv) in CH₂Cl₂ (150 mL) at 23° C. was added AgOAc (2.47 g, 6.33 mmol, 3.50 equiv). The suspension was stirred at 40° C. for 3 h. After cooling to 23° C., the suspension was filtered through a plug of Celite. The filtrate was concentrated in vacuo and the residue was triturated with Et₂O (100 mL). The solid was filtered off and washed with Et₂O (2×50 mL) to afford 2.48 g of the title compound as a yellow solid (99% yield).

¹H NMR (500 MHz, CDCl₃, 23° C., δ): 8.95 (d, J=5.7 Hz, 2H), 8.66 (d, J=4.8 Hz, 1H), 8.01 (d, J=7.6 Hz, 1H), 7.85 (d, J=7.6 Hz, 1H), 7.82 (t, J=7.6 Hz, 1H) 7.76-7.70 (m, 3H), 7.42-7.36 (m, 3H), 7.27 (dd, J=7.6 Hz, J=5.7 Hz, 1H), 7.13 (d, J=7.6 Hz, 2H), 6.45 (d, J=8.6 Hz, 2H), 1.94 (s, 3H); 1.80 (s, 3H); ¹³C NMR (125 MHz, CDCl₃, 23° C., δ): 177.4, 151.7, 151.2, 141.8, 140.5, 139.5, 139.1, 138.5, 138.0, 135.9, 130.2, 130.0, 129.0, 127.8, 127.3, 126.7, 126.3, 125.6, 124.9, 124.0, 121.7, 23.8, 21.0. These spectroscopic data correspond to the reported data in reference¹².

### Aryl Palladium Complex 17

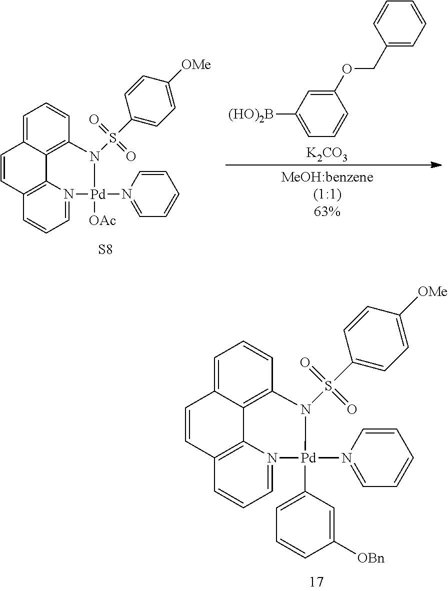

To acetato palladium complex (SS) (1.00 g, 1.64 mmol, 1.00 equiv) in MeOH (20 mL) and benzene (20 mL) at 23° C. was added (3-benzyloxyphenyl)boronic acid (0.490 g, 2.14 mmol, 1.30 equiv) and K₂CO₃ (0.450 g, 3.29 mmol, 2.00 equiv). The reaction mixture was stirred at 23° C. for 18 h, and the solvent was removed in vacuo. To the solid residue was added CH₂Cl₂ (50 mL) and water (50 mL). The phases were separated and the aqueous phase was extracted with CH₂Cl₂ (3×30 mL). The combined organic phases were washed with brine (10 mL) and dried (Na₂SO₄). The filtrate was concentrated in vacuo and the residue was purified by chromatography on silica gel eluting with hexane/EtOAc 1:5 (v/v) to afford 0.760 g of the title compound as a light yellow solid (63% yield).

TLC (hexane/EtOAc 1:5, v/v): R_{F}=0.40; ¹H NMR (500 MHz, CDCl₃, 23° C., δ): 9.00 (d, J=4.9 Hz, 2H), 8.33 (dd, J=5.5 Hz, J=1.2 Hz, 1H), 7.94 (d, J=7.9 Hz, 1H), 7.73-7.60 (m, 5H), 7.36 (d, J=8.5 Hz, 1H), 7.31-7.23 (m, 7H), 7.13 (d, J=8.5 Hz, 2H), 6.99 (dd, J=7.3 Hz, J=5.5 Hz, 1H), 6.72 (t, J=7.6 Hz, 1H), 6.58 (d, J=1.8 Hz, 1H), 6.54 (d, J=7.3 Hz, 1H), 6.47 (dd, J=7.9 Hz, J. 1.8 Hz, 1H), 6.18 (d, J. 8.5 Hz, 2H), 4.89 (d, J=12.2 Hz, 1H), 4.83 (d, J=12.2 Hz, 1H), 3.53 (s, 3H); ¹³C NMR (125 MHz, CDCl₃, 23° C., δ): 160.1, 158.1, 156.4, 153.8, 153.2, 144.7, 143.4, 137.6, 137.5, 136.2, 136.1, 130.0, 129.8, 128.5, 127.7, 127.7, 127.6, 127.4, 127.3, 127.1, 127.1, 124.8, 124.1, 123.5, 121.1, 120.7, 112.2, 109.9, 69.5, 55.2. Anal: calcd for C₃₈H₃₁N₃O₄PdS: C, 62.34; H, 4.27; N, 5.74. found: C, 62.42; H, 4.19; N, 5.72. X-ray quality crystals were obtained from the saturated MeOH solution of the title compound at 23° C. For crystallography data, see X-ray section.

### Aryl Palladium Complex SF

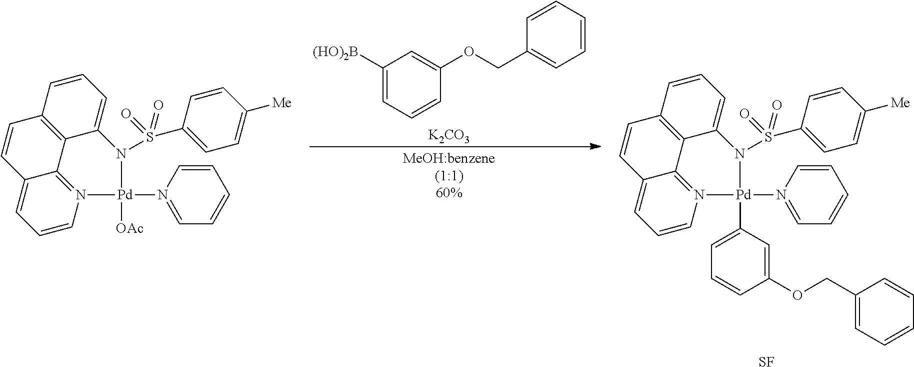

To acetato palladium complex (SE) (500 mg, 0.850 mmol, 1.00 equiv) in MeOH (20 mL) and benzene (20 mL) at 23° C. was added (3-benzyloxyphenyl)boronic acid (212 mg, 0.930 mmol, 1.10 equiv) and K₂CO₃ (233 mg, 1.69 mmol, 2.00 equiv). The reaction mixture was stirred at 23° C. for 24 h, and the solvent was removed in vacuo. To the solid residue was added CHCl₃ (10 mL) and water (10 mL). The phases were separated and the aqueous phase was extracted with CHCl₃ (3×10 mL). The combined organic phases were washed with brine (10 mL) and dried (Na₂SO₄). The filtrate was concentrated in vacuo and the residue was purified by chromatography on silica gel eluting with hexane/EtOAc 1:2 (v/v) to afford 360 mg of the title compound as a light yellow solid (60% yield).

TLC (hexane/EtOAc 1:2, v/v): R_{F}=0.35; ¹H NMR (500 MHz, CDCl₃, 23° C., δ): 9.01 (d, J=4.8 Hz, 2H), 8.29 (d, J=6.7 Hz, 1H), 7.94 (d, J=6.7 Hz, 1H), 7.76-7.63 (m, 5H), 7.38 (d, J=9.5 Hz, 1H), 7.32-7.27 (m, 7H), 7.07 (d, J=7.6 Hz, 2H), 7.00 (dd, J=7.6 Hz, J=5.7 Hz, 1H), 6.72 (t, J=7.6 Hz, 1H), 6.56 (d, J=2.9 Hz, 1H), 6.52 (d, J=7.6 Hz, 1H), 6.48 (d, J=7.6 Hz, 3H), 4.90 (d, J=12.4 Hz, 1H), 4.84 (d, J=12.4 Hz, 1H), 2.00 (s, 3H); ¹³C NMR (125 MHz, CDCl₃, 23° C., δ): 158.1, 156.5, 153.8, 153.3, 144.7, 143.4, 141.1, 139.4, 137.6, 137.4, 136.3, 130.0, 129.8, 128.6, 127.9, 127.8, 127.7, 127.6, 127.5, 127.3, 127.2, 127.1, 126.0, 124.8, 124.1, 123.6, 121.0, 120.8, 110.0, 69.6, 21.1. Anal: calcd for C₃₈H₃₁N₃O₃PdS: C, 63.73; H, 4.36; N, 5.87. found: C, 63.48; H, 4.50; N, 5.82.

### Aryl Palladium Complex 12

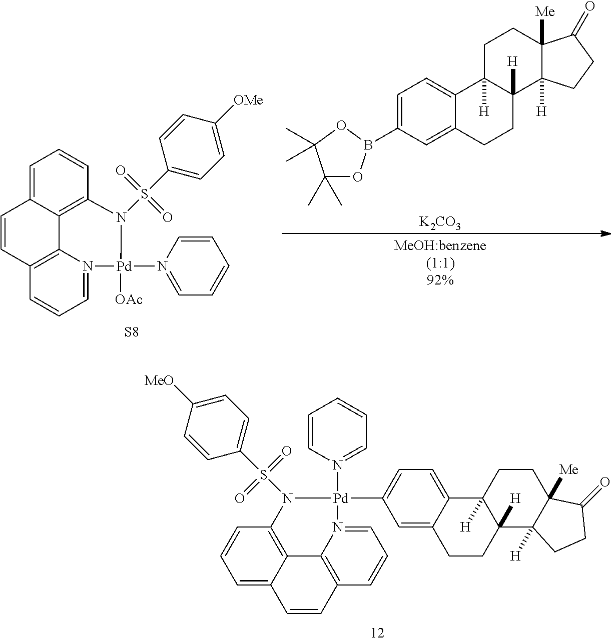

3-Pinacolatoboroestra-1,3,5-(10)-triene-17-one was prepared by slight modification of a published method¹³: To a mixture of 3-(trifluoromethanesulfonyl)estrone¹⁴ (11.0 g, 27.3 mmol, 1.00 equiv) and Pd(dppf)Cl₂—CH₂Cl₂ (1.12 g, 1.37 mmol, 0.0500 equiv) in dioxane (100 ml) under nitrogen atmosphere were added Et₃N (22.9 ml, 164 mmol, 6.00 equiv) and 4,4,5,5-tetramethyl-1,3,2-dioxaborolane (11.5 ml, 79.0 mmol, 2.89 equiv). The reaction mixture was heated at 100° C. for 20 h while stirring. The reaction was cooled to 23° C. and concentrated in vacuo. The residue was dissolved in a 1:3 solution EtOAc:hexanes (100 mL) and the solution was filtered though silica gel to remove palladium residues. The filtrate was concentrated in vacuo and the residue was washed with cold (−15° C.) pentane (3×10 mL) to afford 8.30 g of 3-pinacolatoboroestra-1,3,5-(10)-triene-17-one as a colorless solid (80% yield). ¹H NMR (500 MHz, CDCl₃, 23° C., δ): 7.60 (d, J=7.8 Hz, 1H), 7.57 (s, 1H), 7.32 (d, J=7.8 Hz, 1H), 2.95-2.88 (m, 2H), 2.53-2.44 (m, 2H), 2.35-2.29 (m, 1H), 2.18-1.95 (m, 4H), 1.67-1.40 (m, 6H), 1.34 (s, 12H), 0.91 (s, 3H). ¹³C NMR (125 MHz, CDCl₃, 23° C., δ): 220.8, 143.2, 135.9, 135.6, 132.2, 124.8, 83.7, 50.6, 48.0, 44.8, 38.1, 35.9, 31.7, 29.2, 26.5, 25.7, 24.9, 24.9, 21.7, 13.9. ¹³ V. Ahmed, Y. Liu, C. Silvestro, S. D. Taylor, Bioorg. Med. Chem. 14, 8564 (2006).¹⁴ T. Furuya, A. E. Strom, T. Ritter, J. Am. Chem. Soc. 131, 1662 (2009)

To acetato palladium complex (S8) (1.00 g, 1.64 mmol, 1.00 equiv) in MeOH (30 mL) and benzene (30 mL) at 23° C. was added 3-pinacolatoboroestra-1,3,5-(10)-triene-17-one (0.625 g, 1.64 mmol, 1.00 equiv) and K₂CO₃ (0.340 g, 2.47 mmol, 1.50 equiv). The reaction mixture was stirred at 23° C. for 20 h, and the solvent was removed in vacuo. To the solid residue was added CH₂Cl₂ (100 mL) and the solution was filtered through Celite. The solution was washed water (3×20 mL), and the organic phase were dried (Na₂SO₄). The filtrate was concentrated in vacuo and the residue was recrystallized from CH₂Cl₂/pentane to afford 1.22 g of the title compound as a yellow solid (92% yield).

¹H NMR (500 MHz, CDCl₃, 23° C., δ): 9.04 (d, J=5.3 Hz, 2H), 8.37 (t, J=4.3 Hz, 1H), 7.98 (dd, J=7.5 Hz, J=2.1 Hz, 1H), 7.75-7.60 (m, 5H), 7.39 (d, J=8.6 Hz, 1H), 7.31 (t, J=7.0 Hz, 2H), 7.12-7.06 (m, 3H), 6.68 (dd, J=7.5 Hz, J=4.3 Hz, 1H), 6.57-6.49 (m, 2H), 6.18 (d, J=8.6 Hz, 2H), 3.55 (s, 3H), 2.72-2.51 (m, 2H), 2.46 (dd, J=19.1 Hz, 10.8 Hz, 1H), 2.26-2.24 (m, 1H), 2.15-2.06 (m, 2H), 2.02-1.97 (m, 1H), 1.88-1.85 (m, 2H), 1.60-1.22 (m, 6H), 0.86 (s, 3H); ¹³C NMR (125 MHz, CDCl₃, 23° C., δ): 221.3, 160.2, 154.2, 154.2, 153.4, 151.9, 144.9, 143.6, 137.5, 137.4, 136.3, 136.2, 135.3, 135.2, 134.6, 134.5, 132.3, 132.2, 130.1, 129.9, 127.8, 127.7, 127.6, 127.3, 127.2, 124.8, 124.8, 124.1, 123.5, 123.4, 123.4, 121.2, 121.1, 112.2, 55.3, 50.7, 50.7, 48.2, 44.3, 44.3, 38.3, 36.0, 31.8, 29.4, 26.9, 26.8, 25.7, 25.6, 21.7, 14.1, 14.0. Anal: calcd for C₄₃H₄₁N₃O₄PdS: C, 64.37; H, 5.15; N, 5.24. found: C, 64.06; H, 5.21; N, 5.21.

### Example 10

### 3-Benzyloxyphenyl fluoride (20)

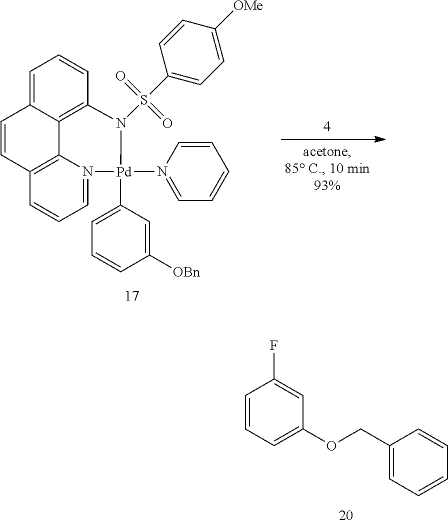

In a glove box, palladium aryl complex 17 (100 mg, 0.140 mmol, 1.00 equiv) was dissolved in acetone (7 mL) and added to a soda lime glass bottle charged with Pd(IV)-F complex 2 (102 mg, 0.140 mmol, 1.00 equiv). The bottle was sealed, taken out of the glove box, and immersed in an oil bath heated at 85° C. for 10 minutes. The reaction mixture was cooled and concentrated in vacuo. The volatiles were removed in vacuo and the residue was extracted with Et₂O (5×5 mL). The extract was concentrated in vacuo and the residue was purified by chromatography on silica gel eluting with hexane/EtOAc 10:1 (v/v) to afford 25.6 mg of the title compound as a colorless oil (93% yield).

TLC (hexane/EtOAc 20:1, v/v): R_{F}=0.55; NMR Spectroscopy: ¹H NMR (500 MHz, CDCl₃, 23° C., δ): 7.44-7.33 (m, 5H), 7.23 (q, J=8.6 Hz, J=6.7 Hz, 1H), 6.77 (dd, J=8.6 Hz, J=2.9 Hz, 1H), 6.72-6.66 (m, 2H), 5.06 (s, 2H). ¹³C NMR (125 MHz, CDCl₃, 23° C., δ): 163.8 (d, J=246 Hz), 160.3 (d, J=11 Hz), 136.6, 130.4 (d, J=10 Hz), 128.8, 128.3, 127.6, 110.8 (d, J=3 Hz), 107.9 (d, J=22 Hz), 102.8 (d, J=25 Hz), 70.4. ¹H NMR (375 MHz, CD₃CN, 23° C., δ): −112.2 (m). These spectroscopic data correspond to the reported data¹⁵. ¹⁵ D. A. Watson et al., Science 321, 1661 (2009).

### 3-Benzyloxyphenyl fluoride (20)

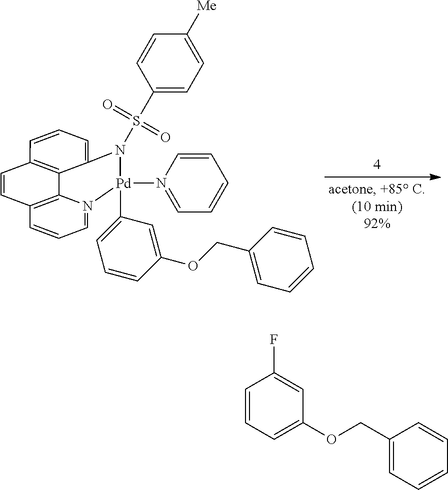

In a glove box, palladium aryl complex SF (102 mg, 0.140 mmol, 1.00 equiv) was dissolved in acetone (7 mL) and added to a soda lime glass bottle charged with Pd(IV)-F complex 4 (100 mg, 0.140 mmol, 1.00 equiv). The bottle was sealed, taken out of the glove box, and immersed in an oil bath heated at 85° C. for 10 minutes. The reaction mixture was cooled and concentrated in vacuo. The volatiles were removed in vacuo and the residue was extracted with Et₂O (5×5 mL). The extract was concentrated in vacuo and the residue was purified by chromatography on silica gel eluting with hexane/EtOAc 10:1 (v/v) to afford 25.9 mg of the title compound as a colorless oil (92% yield).

TLC (hexane/EtOAc 20:1, v/v): R_{F}=0.55; NMR Spectroscopy: ¹H NMR (500 MHz, CDCl₃, 23° C., δ): 7.45-7.34 (m, 5H), 7.24 (q, J=8.6 Hz, J=6.7 Hz, 1H), 6.78 (dd, J=8.6 Hz, J=2.9 Hz, 1H), 6.73-6.67 (m, 2H), 5.07 (s, 2H). ¹⁹F NMR (375 MHz, CD₃CN, 23° C., δ): −112.16 (m). ¹³C NMR (125 MHz, CDCl₃, 23° C., δ): 163.84 (d, J=246.2 Hz)−160.33 (d, J=11.0 Hz), 136.69, 130.43 (d, J=10.1 Hz), 128.86, 128.34, 127.70, 110.85 (d, J=2.9 Hz), 107.97 (d, J=21.6 Hz), 102.84 (d, J=24.9 Hz), 70.46. These spectroscopic data correspond to the reported data¹⁵.

### 3-Deoxy-3-fluoroestrone (21)

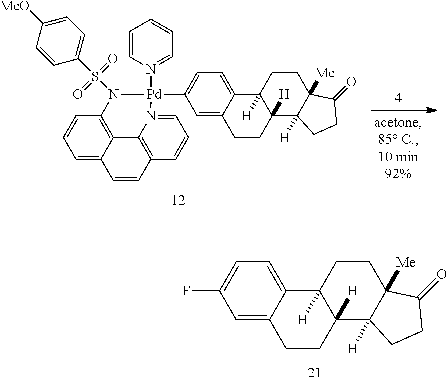

In a glove box, palladium aryl complex 12 (100 mg, 0.124 mmol, 1.00 equiv) was dissolved in acetone (7 mL) and added to a soda lime glass bottle charged with Pd(IV)-F complex 4 (90.7 mg, 0.124 mmol 1.00 equiv). The bottle was sealed, taken out of the glove box, andimmersed in an oil bath heated at 85° C. for 10 minutes. The reaction mixture was cooled and concentrated in vacuo. The volatiles were removed in vacuo and the residues were extracted with Et₂O (5×5 mL). The extract was concentrated in vacuo and the residue was purified by chromatography on silica gel eluting with hexane/EtOAc 10:1 (v/v) to afford 31.1 mg of the title compound as a colorless solid (93% yield).

R_{f}=0.33 (hexane/EtOAc 9:1 (v/v)). NMR Spectroscopy: ¹H NMR (500 MHz, CDCl₃, 23° C., 8): 7.23 (dd, J=8.5 Hz, 3.2 Hz, 1H), 6.83 (td, J. 8.5 Hz, 2.1 Hz, 1H), 6.79 (dd, J=9.6 Hz, 3.2 Hz, 1H), 2.92-2.88 (m, 2H), 2.51 (dd, J. 19.0 Hz, 9.0 Hz, 1H), 2.42-2.38 (m, 1H), 2.29-2.23 (m, 1H), 2.18-1.94 (m, 4H), 1.67-1.41 (m, 6H,), 0.91 (s, 3H). ¹³C NMR (125 MHz, CDCl₃, 23° C., δ): 220.8, 161.2 (d, J=244 Hz), 138.8 (d, J=6 Hz), 135.5 (d, J=3 Hz), 126.9 (d, J=8 Hz), 115.3 (d, J=20 Hz), 112.6 (d, J=21 Hz), 50.5, 48.1, 44.1, 38.3, 36.0, 31.7, 29.6, 26.5, 26.0, 21.7, 14.0. ¹⁹F NMR (375 MHz, CDCl₃, 23° C., δ):-117.1. These spectroscopic data correspond to previously reported data¹⁶. ¹⁶ P. Tang, T. Furuya, T. Ritter, J. Am. Chem. Soc. 132, 12150 (2010).

### Example

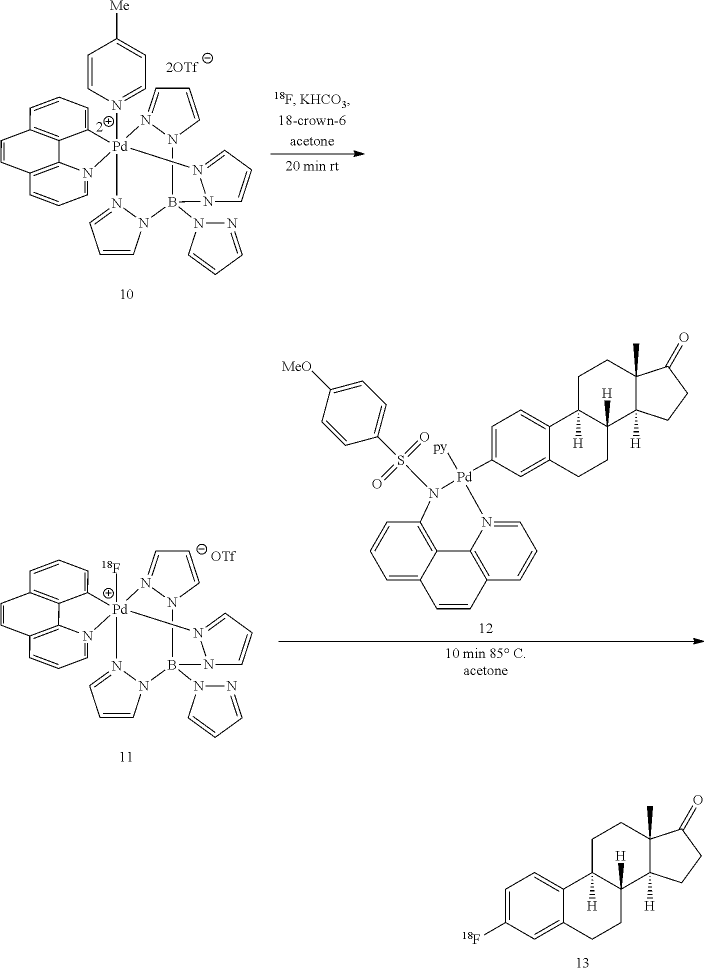

[¹⁸F]Fluoride solution obtained from a cyclotron was loaded onto a Macherey-Nagel SPE cartridge Chromafix 30-PS—HCO₃ cartridge that had been previously washed with 2 mL of 5 mg/mL KHCO₃ in Millipore water and 20 mL of Millipore Milli-Q water. After loading, the cartridge was washed with 2 mL of Millipore Milli-Q water. [¹⁸F]Fluoride was eluted with 2 mL of a 5 mg/mL KHCO₃ in Millipore Milli-Q water solution. The solution was diluted with 8 mL of acetonitrile providing 10 mI, of 4:1 MeCN:H₂O solution containing 1 mg/mL KHCO₃. 1 mL of this solution was then put in a magnetic-stir-bar-containing conical vial that had been washed with acetone, deionized water, sodium hydroxide/ethanol solution, and deionized water, and dried at 150° C. prior to use. 0.5 mL of a stock solution containing 18-crown-6 (26.2 mg/mL MeCN) was then added. The solution was evaporated at 108° C. with a constant nitrogen gas stream. At dryness 0.5 mL of acetonitrile was added and evaporated at 108° C. with a constant nitrogen gas stream. Another 0.5 mL of acetonitrile was added and evaporated at 108° C. with a constant nitrogen gas stream to leave a white precipitate around the bottom and sides of the vial. 0.5 mL of acetone was added and evaporated to dryness at 108° C. with a constant nitrogen gas stream to leave a glassy film on the bottom and sides of the vial. The vial was cooled in a water bath, purged with nitrogen, and sealed with a cap fitted with a septum. First step: 10 mg of Pd(IV) complex 10 dissolved in 0.5 mL of acetone was added via the septum. The vial was sonicated and then allowed to stir at 23° C. for 10 minutes. During this time, the orange/brown clear solution became opaque. At the end of 10 minutes, the vial was opened and the suspension was loaded with a glass pipette into another glass pipette containing a small amount of cotton and 25 mg of JandaJel™-polypyridine that had been suspended in 0.3 mL of acetone for 15 minutes prior to loading the solution. The vial was washed with 0.5 mL of acetone and the solution was added onto the JandaJel™-polypyridine. At this point the solution was fully pushed through the JandaJel™-polypyridine and cotton with air into a new 1 dram vial equipped with a magnetic stir bar. At this point, the solution was less opaque. An additional 0.5 mL of acetone was used to wash the conical vial. The solution was added onto the JandaJel™-polypyridine and pushed through with air into the 1 dram vial.

Second step: To the 1.5 mL acetone solution was added 10 mg of the Pd(II) aryl complex. The vial was capped securely, and the mixture heated at 85° C. After 10 minutes the solution was cooled. A capillary tube was used to spot the solution on silica gel TLC plate. The TLC plate was emerged in an appropriate organic solvent mixture. The TLC plate was scanned with a Bioscan AR-2000 Radio TLC Imaging Scanner to determine radioactive products.

### Measurement of Radiochemical Yield

Radiochemical yield was determined by multiplying the percentage of radioactivity in the final solution and the relative peak integrations of a radio TLC scan. First, the radioactivity of the 1.5 mL solution collected after filtration was measure in a an ion chamber, followed by the amount of radioactivity on the JandaJel™-polypyridine/cotton pipette and the amount of radioactivity left on the walls of the initial conical vial. These measurements determined the fraction of radioactivity that entered the second step (typically 55-75%). After the second step was complete the solution was transferred to a second 1 dram vial using an additional 0.5 mL acetone wash and the amount of radioactivity in this solution was measured. The amount of radioactivity on the original 1 dram vial was then measured to determine the percentage of radioactivity of the solution that spotted onto the TLC plate (typically 90%). After radio TLC quantification, the radiochemical yield was determined by multiplying the product quantified during TLC by the fraction of radioactivity in solution over two steps (typically 50-70%). For example, Entry 1 of Radiochemical Yield Data section:

Measured radioactivity in 1.5 mL acetone solution after first step: 256 μCi

Measured radioactivity of pipette containing JandaJel™-polypyridine and cotton: 30 μCi.

Measured radioactivity of conical vial from first step: 26 μCi

Radioactivity percentage that entered second step: 82% ((256+30+26)/256*100)

Measured radioactivity of acetone solution after second step: 215 μCi

Measured radioactivity of dram vial from second step: 16 μCi

Radioactivity percentage from second step that was spotted on to TLC plate: 93%

((215+16)/215*100)

Percent ¹⁸F in solution: 76% (0.82*0.93*100)

Radio TLC yield (RTLC yield): 43%

Radiochemical yield (RCY): 33% (0.76*0.43*100)

TABLE S1
Radiochemical Yield Data

| | | RTLC | % ¹⁸F in | | Average |
|---|---|---|---|---|---|
| Entry | Molecule | yield | solution | RCY | RCY |
| 1 | [¹⁸F]21 | 43 | 76% | 33 | |
| 2 | [¹⁸F]21 | 51 | 68% | 35 | |
| 3 | [¹⁸F]21 | 36 | 76% | 27 | |
| 4 | [¹⁸F]21 | 43 | 69% | 30 | |
| 5 | [¹⁸F]21 | 58 | 68% | 40 | |
| 6 | [¹⁸F]21 | 57 | 66% | 38 | |
| 7 | [¹⁸F]21 | 49 | 57% | 28 | |
| 8 | [¹⁸F]21 | 44 | 66% | 29 | 33 |

### Characterization of ¹⁸F-labeled Molecules

All ¹⁸F-labeled molecules were characterized by 1) comparing the radioactivity trace of the crude reaction mixture to the UV trace of authentic reference sample and 2) comparing the TLC Rf of radioactive products to the Rf of authentic reference sample in two different TLC solvent mixtures. An Agilent Eclipse XDB-C18, 5 μm, 4.6×150 mm HPLC column was used for analytical HPLC analysis. Analytical HPLC used the following mobile phases: 0.1% CF₃CO₂H in water (A) 0.1% CF₃CO₂H in acetonitrile (B). Program: 50% (B) for 2 minutes then a gradient 50-95% (B) over 8 minutes. Note: radioactivity chromatographs have been offset (−0.125 min) to account for the delay volume (time) between the diode array detector and the radioactivity detector.

### Evidence for Formation of [¹⁸F]4 During Radiochemical Experiments

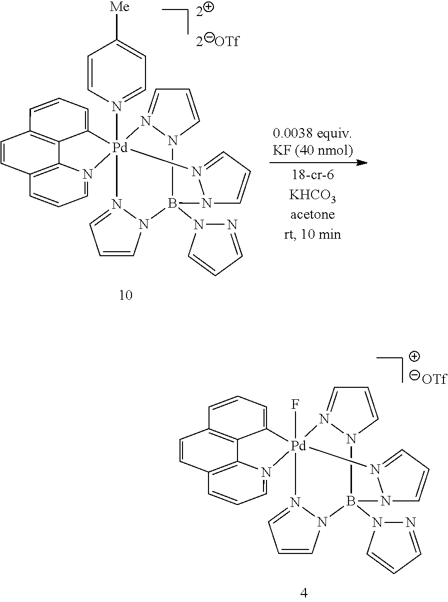

To provide evidence for the formation of [¹⁸F]4 during radiochemical experiments, PET conditions were mimicked using only [¹⁹F]fluoride in order to observe 4 directly by NMR spectroscopy. Typical specific radioactivity of [¹⁸F]fluoride from the nuclear reaction ¹⁸O(p,n)¹⁸F is 5 Ci/μmol so that 1 Ci of ¹⁸F contains 200 nmol of total fluoride (0.6 nmol of [¹⁸F]fluoride)¹⁷. Based on this, we used 40 nmol of KF, to mimic a 200 mCi sample at 5 Ci/μmol. Complex 4 was identified during the experiment by ¹⁹F NMR spectroscopy (Figure S10). The direct observation of 4 shows that the complex is stable relative to other reagents such as 18-cr-6, KHCO₃, 10 as well as H₂O in reaction conditions used for the described radiochemistry. ¹⁷ R. Ting, M. J. Adam, T. J. Ruth, D. M. Perrin, J. Am. Chem. Soc. 127, 13094 (2005).

Experimental: 1.0 mL of a 4:1 MeCN:H₂O solution containing 1.0 mg KHCO₃ was transferred to a conical vial that contained KF (0.0023 mg, from a stock solution 1.0 mg/mL KF) and a magnetic stir bar. 0.5 mL of a stock solution containing 18-crown-6 (26.2 mg/mL MeCN) was then added. The solution was evaporated at 108° C. with a constant nitrogen gas stream. At dryness 0.5 mL of acetonitrile was added and evaporated at 108° C. with a constant nitrogen gas stream. Another 0.5 mL of acetronitrile was added and evaporated at 108° C. with a constant nitrogen gas stream to leave a white precipitate around the bottom and sides of the vial. 0.5 mL of acetone was added and evaporated to dryness at 108° C. with a constant nitrogen gas stream to leave a glassy film on the bottom and sides of the vial. The vial was cooled in a water bath, purged with nitrogen, and sealed with a cap fitted with a septum. 10 mg of 10 dissolved in 0.6 mL of d₆-acetone was added via the septum. The vial was sonicated for 20 seconds and then allowed to stir at 23° C. for 10 minutes. During this time, the orange/brown clear solution became opaque. At the end of 10 minutes, the solution was transferred to a NMR tube and analyzed by ¹⁹F NMR spectroscopy (Figure S10).

### Automated Synthesis of [¹⁸F]21 and Use of High Specific Activity Fluoride

In order to demonstrate the successful application of our method to high specific activity [¹⁸F]fluoride and a large radioactivity scale using an automated synthesis module, an approximately 1 Ci-scale experiment was performed. 0.0609 μmole of 21 was made. The radioactivity of the sample was 0.0620 Ci at the end of synthesis time. Specific activity=1.03 Ci/μmmol (38.1 GBq/μmmol). The experiment was accomplished using an Eckert and Ziegler automated synthesis module and Modular-Lab. 10% of the final acetone solution was injected on a preparative Waters Bondapak™ C18 column using an eluent of 40:60 (v/v) MeCN:H₂O with 0.1% CF₃CO₂H. 10% was purified to avoid an unnecessary radioactivity dose. The radioactive fraction corresponding to [¹⁸F]21 was collected. The fraction was loaded onto a Waters Sep-Pak® Plus C18 cartridge, eluted with MeCN, and concentrated to 1.0 mL. 0.1 mL of the solution was analyzed by HPLC on an Agilent Eclipse XDB-C18 analytical column using the gradient method described above. The UV absorbance (corresponding to 1% of sample) was compared to a standard curve of UV absorbance versus nmoles of authentic 21. The standard curve was generated by integration of the UV absorbance signal (at 280 nm) of 4 different known amounts of 21 in duplicate (see Figure S11).

### DFT Computations

Density functional theory (DFT) calculations were performed using Gaussian09¹⁸ at the Odyssey cluster at Harvard University. Geometry optimizations were carried out using the atomic coordinates of the crystal structure benzo[h]quinolinyl (tetrapyrazolylborate) Pd(IV) pyridine trifluoromethanesulfonate (SB) and benzo[h]quinolinyl (tetrapyrazolylborate) Pd(IV) fluoride trifluoromethanesulfonate (2) as starting points with the B3PW9^{19,20} hybrid functional. The unrestricted wave function was used for the singlet ground state of SB and 4. BS I includes SDD quasirelativistic pseudopotentials on Pd (28) with basis sets (Pd: (8s7p6d)/[6s5p3d]^{21,22}) extended by polarization functions (Pd: f, 1.472²³), and 6-31G(d,p)²⁴ on H, B, C, N, F. All geometry optimizations were performed using the B3PW91 with the BS I basis set, followed by frequency calculations on each optimized structure with corresponding functional/BS I. Molecular orbitals of SB and 4 were generated using an isosurface value of 0.03 on the optimized structure of SB and 4 with B3PW91/BS I. NBO ¹⁸ M. J. Frisch et al., Gaussian 09, Revision A.02 (Gaussian, Inc., Wallingford Conn., 2009).¹⁹A. D. Becke, J. Chem. Phys. 98, 5648 (1993).²⁰ J. P. Perdew, Y. Wang, Phys. Rev. B 45, 13244 (1992).²¹ D. Andrae et al., Theor. Chim. Acta 77, 123 (1990).²² D. Andrae et al., Theor. Chim. Acta 78, 247 (1991).²³A. W. Ehlers et al., Chem. Phys. Lett. 208, 111 (1993).²⁴ P. C. Hariharan, J. A. Pople, Theor. Chim. Acta 28, 213 (1973). analysis²⁵ was done with BS I. Lowest unoccupied molecular orbital (LUMO) of 2 was generated using an isosurface value of 0.03 on the optimized structure of 2 with B3PW91/BS I.²⁵ (a) E. D. Glendening, A. E. Reed, J. E. Carpenter, F Weinhold, NBO, Version 3.1. (b) A. E. Reed, L. A. Curtiss, F. Weinhold, Chem. Rev. 88, 899 (1988).

### Example 12

X-Ray Crystallography: A crystal mounted on a diffractometer was collected data at 100 K. The intensities of the reflections were collected by means of a Bruker APEX II CCD diffractometer (Mo_{Kα} radiation, λ=0.71073 Å), and equipped with an Oxford Cryosystems nitrogen flow apparatus. The collection method involved 0.5° scans in ω at 28° in 2θ. Data integration down to 0.75 Å resolution (10) and 0.82 Å resolution (4 and 17), was carried out using SAINT V7.46 A (Bruker diffractometer, 2009) with reflection spot size optimization. Absorption corrections were made with the program SADABS (Bruker diffractometer, 2009).²⁶ The structure was solved by the direct methods procedure and refined by least-squares methods again F² using SHELXS-97 and SHELXL-97 (Sheldrick, 2008).²⁷ Non-hydrogen atoms were refined anisotropically, and hydrogen atoms were allowed to ride on the respective atoms. Crystal data as well as details of data collection and refinement are summarized in Table 2, and geometric parameters are shown in Table 3. The Ortep plots produced with SHELXL-97 program, and the other drawings were produced with Accelrys DS Visualizer 2.0 (Accelrys, 2007). ²⁶ Bruker AXS (2009). APEX II. Bruker AXS, Madison, Wis. ²⁷ G. M. Sheldrick, Acta Cryst. A64, 112 (2008).

Benzo[h]quinolinyl (tetrapyrazolylborate) Pd(IV) pyridine trifluoromethanesulfonate (SB)

The asymmetric unit was found to contain one benzo[h]quinolinyl (tetrapyrazolylborate) Pd(IV) pyridine, two trifluoromethanesulfonate, one acetonitrile, and 0.5 diethyl ether molecules. The acetonitrile molecule was found in two independent locations and was assigned site occupancy factors of 0.5. The diethyl ether molecule was assigned site occupancy factors of 0.5. These assignments were confirmed further by ¹H NMR spectroscopy showing that the single crystals that were dissolved in d₃-MeCN have one acetonitrile molecule and 0.5 diethyl ether molecule per X. One trifluoromethanesulfonate molecule possessed a disordered CF₃ group that was in two positions with site occupancy whose population was determined by X-ray data.

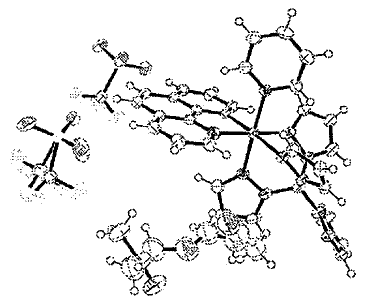

The structure of SB with hydrogen. The nonhydrogen atoms are depicted with 50% probability ellipsoids.

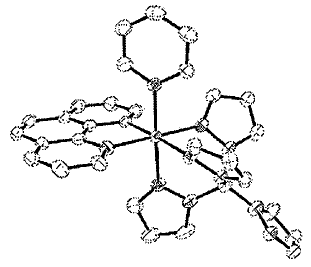

The structure of SB

Benzo[h]quinolinyl (tetrapyrazolylborate) Pd(IV) 4-picoline trifluoromethanesulfonate (10)

The asymmetric unit was found to contain one benzo[h]quinolinyl (tetrapyrazolylborate) Pd(IV) 4-picoline, two trifluoromethanesulfonate, one acetonitrile, and 0.5 diethyl ether molecules. The acetonitrile molecule was found in two different locations and was assigned site occupancy factors of 0.75 and 0.25, respectively. The diethyl ether molecule was assigned site occupancy factors of 0.5. These assignments were confirmed further by ¹H NMR spectroscopy showing that the single crystals that were dissolved in d₃-MeCN have one acetonitrile molecule and 0.5 diethyl ether molecule per 10. One trifluoromethanesulfonate molecule possessed a disordered CF₃ group that was in two positions with site occupancy whose population was determined by X-ray data.

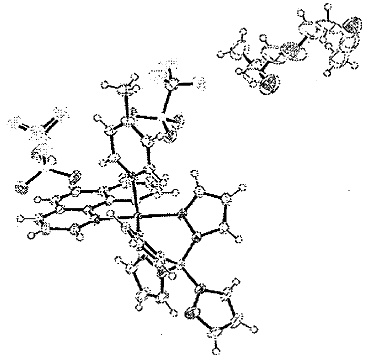

The structure of 10.(MeCN).(Et₂O)_{0.5} with hydrogen. The non-hydrogen atoms are depicted with 50% probability ellipsoids.

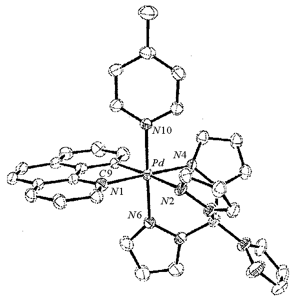

The structure of 10

Benzo[h]quinolinyl (tetrapyrazolylborate) Pd(IV) fluoride trifluoromethanesulfonate (4)

The asymmetric unit was found to contain one benzo[h]quinolinyl (tetrapyrazolylborate) Pd(IV) fluoride and one trifluoromethanesulfonate molecule, respectively.

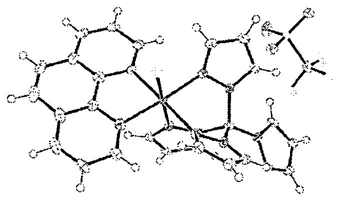

The structure of 4 with hydrogen. The nonhydrogen atoms are depicted with 50% probability ellipsoids.

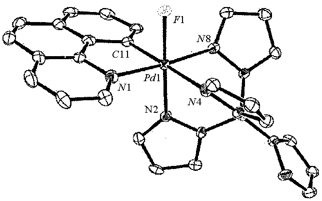

The structure of 4

### Aryl Palladium Complex (17)

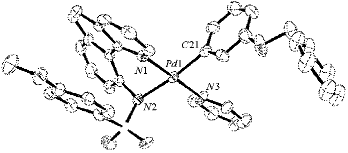

The structure of 17. The nonhydrogen atoms are depicted with 50% probability ellipsoids.

### Other Embodiments

The foregoing has been a description of certain embodiments of the invention. Those of ordinary skill in the art will appreciate that various changes and modifications to this description may be made without departing from the spirit or scope of the present invention, as defined in the following claims.

1. A palladium complex of formula (VII): 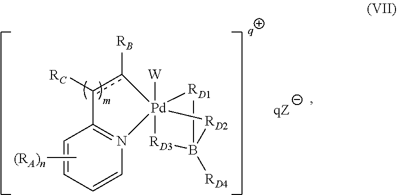
wherein:
the dashed line represents the presence or absence of a bond;
Pd is in the oxidation state +IV;
W is Br, hydroxyl, alkoxy, aryloxy, —NO₃, nitro, —N₃, ClO₄, PO₄, SO₄, —OSO₂-aryl, heteroaryl or heterocyclyl, each of which is substituted with p occurrences of R_{F};
n is 0, 1, 2, 3 or 4;
m is 0, 1, 2 or 3;
p is 0, 1, 2 or 3;
q is 1 or 2;
each occurrence of R_{A} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; or wherein two R_{A} may be taken together with the atoms to which they are attached to form a substituted or unsubstituted carbocyclic, heterocyclic, aryl or heteroaryl ring;
each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted, branched or unbranched heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
each occurrence of R_{C} is independently hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted, heteroaryl; or wherein R_{C} and R_{B} may be taken together with the atoms to which they are attached to form a substituted or unsubstituted heterocyclic or heteroaryl ring or unsubstituted aryl ring; and wherein R_{C} and R_{A} may be taken together with the atoms to which they are attached to form a substituted or unsubstituted carbocyclic, heterocyclic, aryl or heteroaryl ring;
R_{D1}, R_{D2}, R_{D3}, and R_{D4} are each independently cyclic or acyclic, branched or unbranched aliphatic; cyclic or acyclic, branched or unbranched heteroaliphatic; aryl; heteroaryl, each of which is substituted with 0-3 occurrences of R_{H};
each occurrence of R_{H} is independently hydrogen, halogen, alkyl, alkoxy, aryl or heteroaryl;
each occurrence of R_{F} is independently halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an aryl moiety; a heteroaryl moiety; alkoxy; aryloxy; alkylthio; arylthio; amino, alkylamino, dialkylamino, heteroaryloxy; or heteroarylthio moiety; and
Z⁻ is an anion.

2.-11. (canceled)

12. The palladium complex of claim 1, wherein R_{A} and R_{C} taken together with the atoms to which they are attached form an aryl ring and R_{B} and R_{C} taken together with the atoms to which they are attached form an aryl ring.

13.-17. (canceled)

18. The palladium complex of claim 1, wherein R_{D1}, R_{D2}, R_{D3} and R_{D4} are each a 5-membered heteroaryl ring.

19.-53. (canceled)

54. A palladium complex selected from:

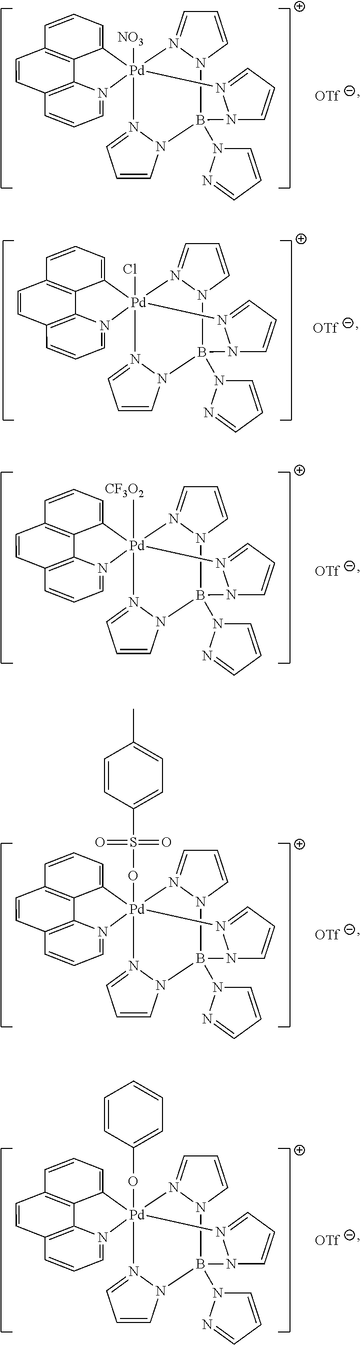

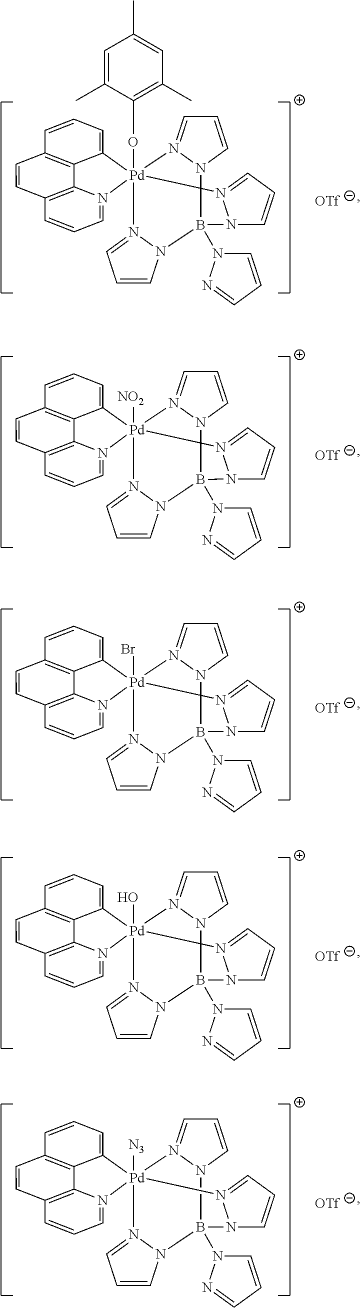

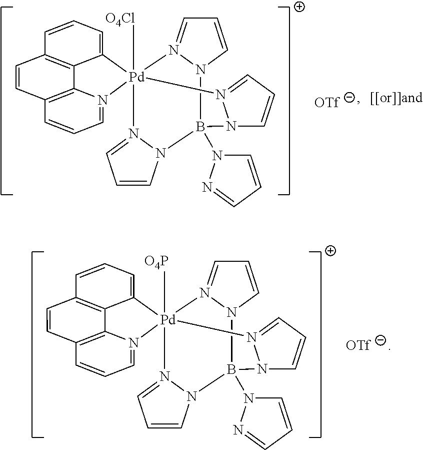

55. The palladium complex of claim 1, wherein the complex is of formula (I): 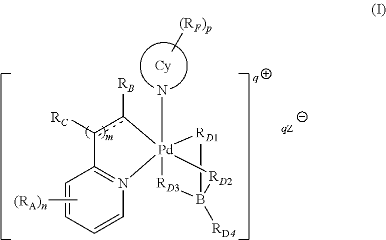
wherein:
Cy taken together with the nitrogen atom to which it is attached forms a heterocyclyl or heteroaryl ring.

56. (canceled)

57. The palladium complex of claim 55, wherein Cy taken together with the nitrogen atom to which it is attached forms a heteroaryl ring.

58.-76. (canceled)

77. The palladium complex of claim 55, wherein the complex is of formula (Ia):

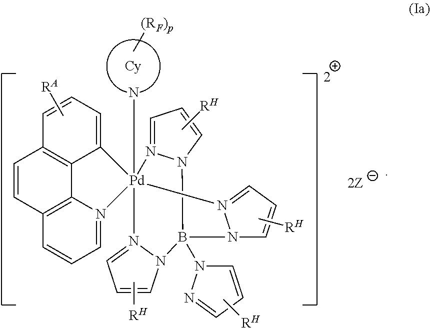

78. The palladium complex of claim 55, wherein the complex is selected from:

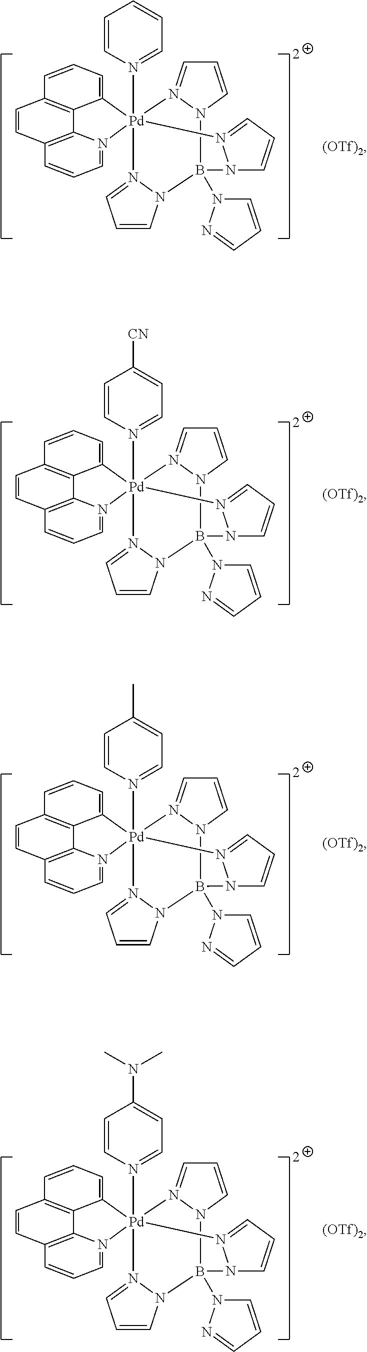

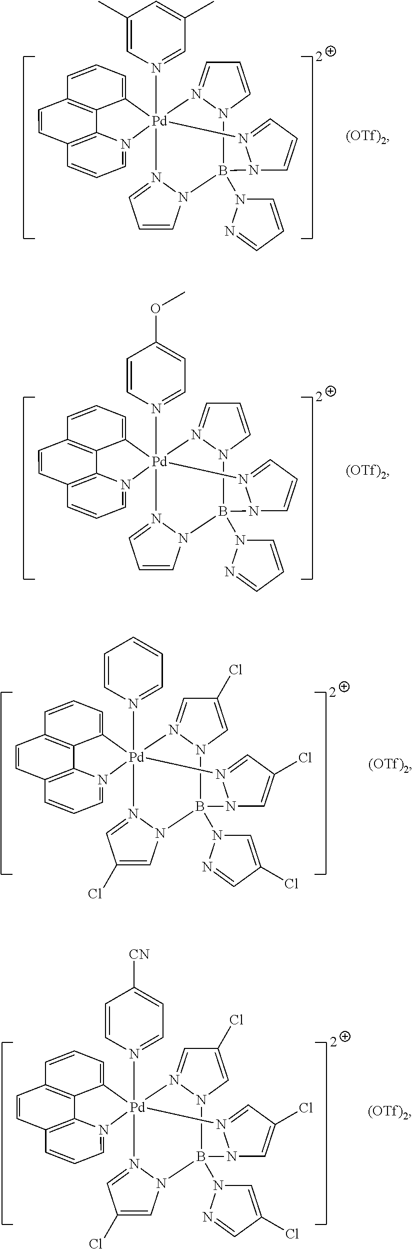

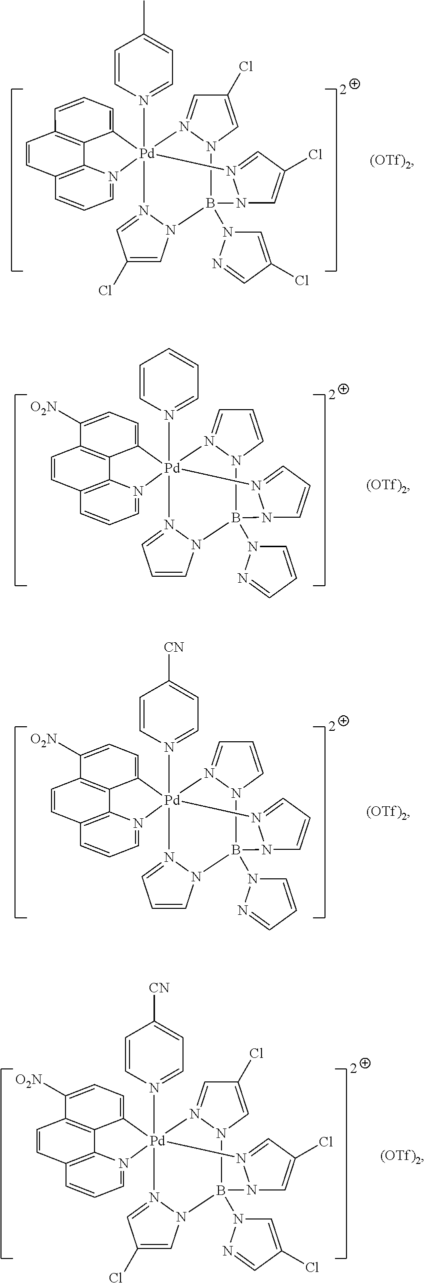

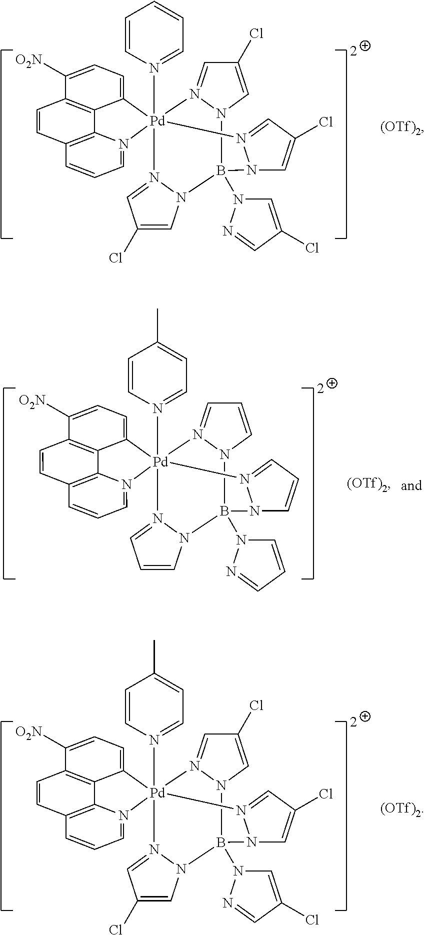

79. (canceled)

80. A palladium complex of formula (II): 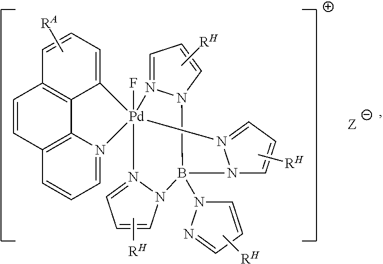
wherein:
R^{A} is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted aryl; substituted or unsubstituted, heteroaryl; —OR′; —C(═O)R′; —CO₂R′; —CN; —SCN; —SR′; —SOR′; —SO₂R′.—NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
each R^{H} is independently selected from hydrogen, halogen, alkyl, alkoxy, aryl or heteoraryl;
F is comprises ¹⁸F or ¹⁹F; and
Z is an anion.

81. (canceled)

82. The palladium complex of claim 80, wherein each R^{H} is hydrogen.

83. (canceled)

84. The palladium complex of claim 80, wherein Z is a halide, acetate, tosylate, azide, tetrafluoroborate, tetraphenylborate, tetrakis(pentafluorophenyl)borate, [B[3,5-(CF₃)₂C₆H₃]₄]⁻, hexafluorophosphate, phosphate, sulfate, perchlorate, trifluoromethanesulfonate or hexafluoroantimonate.

85. (canceled)

86. The palladium complex of claim 80, wherein F comprises ¹⁸F.

87. The palladium complex of claim 80, wherein F comprises ¹⁹F.

88. The palladium complex of claim 80, wherein the complex is selected from:

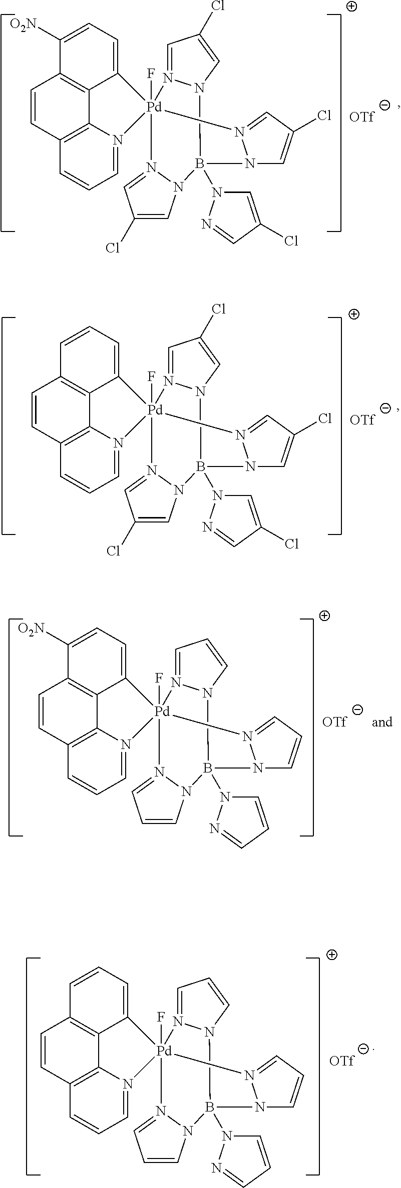

89. The palladium complex of claim 80, wherein the complex is

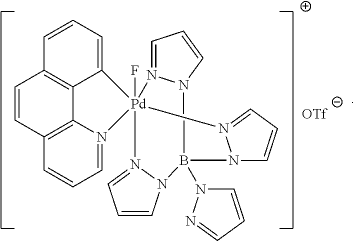

90.-115. (canceled)

116. A method of making the palladium complex of claim 55 of formula (I), the method comprising treating a palladium complex of formula (III): 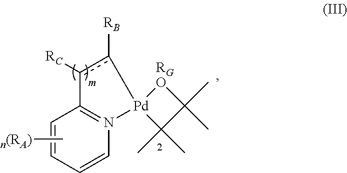
with a borate complex of formula (IV): 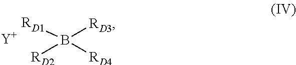
to provide a compound of formula (V): 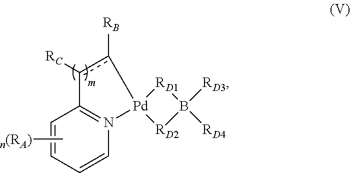
the method further comprising, treating a compound of formula (V) with a compound of formula (VI): 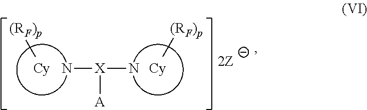
to provide a compound of formula (I), wherein
A is an aryl or heteroaryl group;
R_{G} is acyl;
Y⁺ is a cation; and
X is a halogen

117.-152. (canceled)

153. The method of claim 116, wherein the palladium complex of formula (I) is selected from:

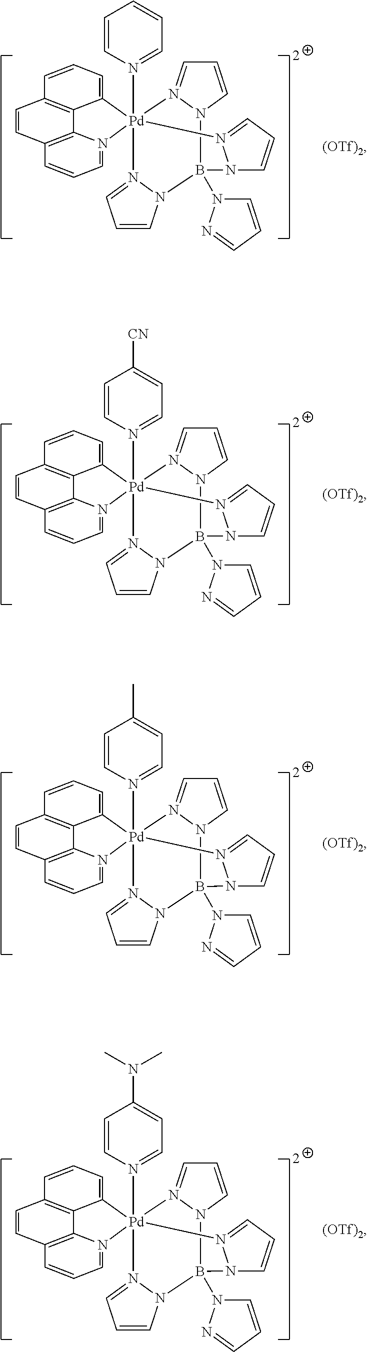

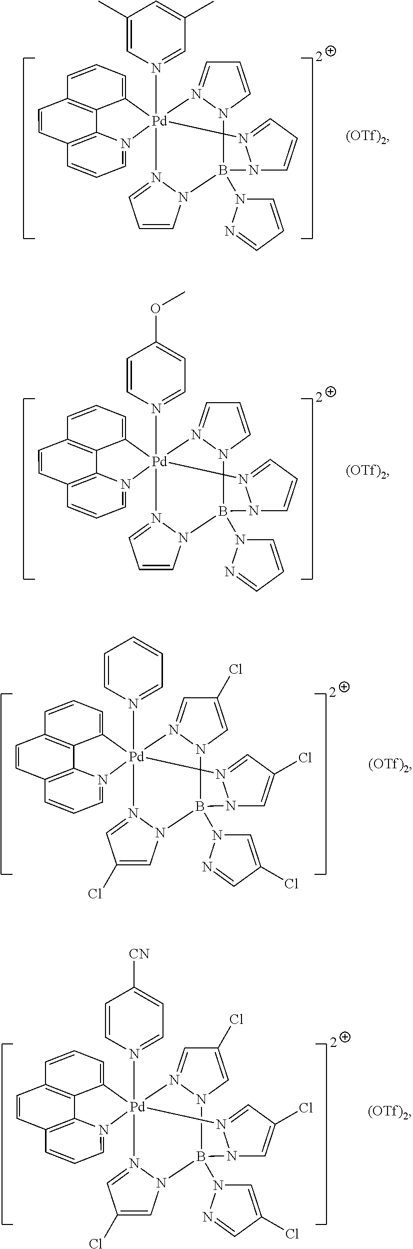

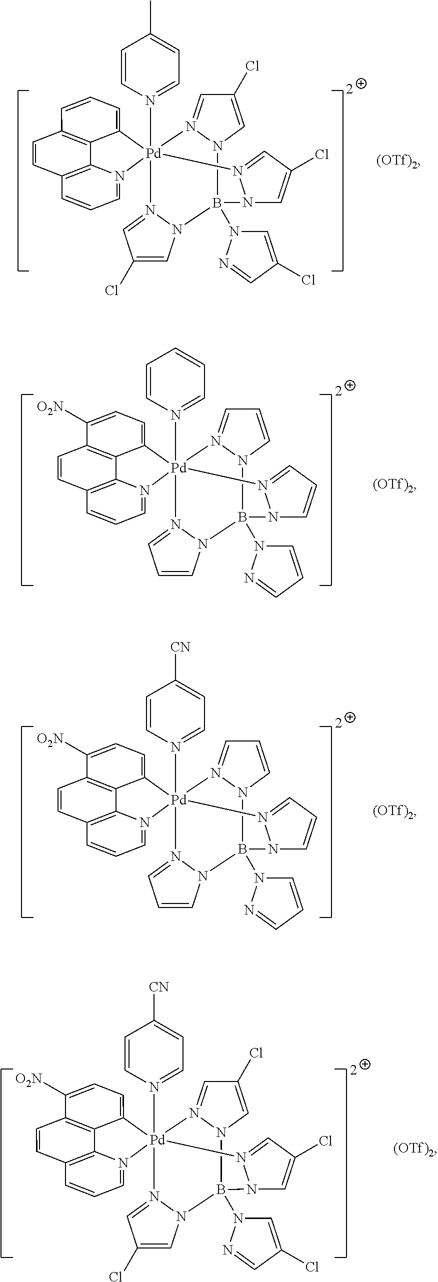

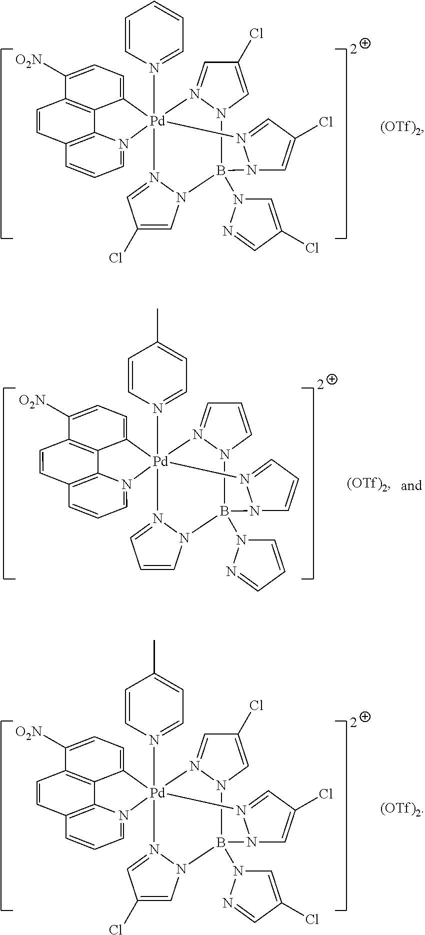

154. (canceled)

155. A method of fluorination, wherein the palladium complex of claim 55 is mixed with F— to produce a palladium (IV) complex and subsequently said palladium (IV) complex is reacted with an organic compound under conditions sufficient to fluorinate the compound, thereby providing a fluorinated organic compound.

156.-157. (canceled)

158. The method of claim 155, wherein F— comprises ¹⁸F—.

159. The method of claim 155, wherein F— comprises ¹⁹F.

160.-250. (canceled)
